(19) United States
(12) Patent Application Publication
(10) Pub. No.: US 2025/0090650 A1
(43) Pub. Date: Mar. 20, 2025

(54) MODIFIED INFLUENZA B HEMAGGLUTININ POLYPEPTIDES AND NUCLEIC ACIDS AND USES THEREOF

(71) Applicant: SANOFI, Paris (FR)

(72) Inventors: Christine BRICAULT, Waltham, MA (US); Gwo-Yu CHUANG, Rockville, MD (US); Raul GOMILA, Waltham, MA (US); Ching-Lin HSIEH, Waltham, MA (US); Andreas KARLSSON, Marcy L'Etoile (FR)

(21) Appl. No.: 18/826,504

(22) Filed: Sep. 6, 2024

(30) Foreign Application Priority Data
Sep. 6, 2023 (EP) 23306481.5

(51) Int. Cl. A61K 39/145; A61K 9/127; A61P 37/04; C07K 14/005

(52) U.S. Cl. CPC A61K 39/145; A61K 9/127; A61P 37/04; C07K 14/005; C12N 2760/16222; C12N 2760/16234; C12N 2760/16252; C12N 2760/16271

(57) ABSTRACT

This application relates to modified influenza B hemagglutinin polypeptides and nucleic acids, such as messenger ribonucleic acids (mRNAs), encoding the same, as well as compositions comprising the same, vaccines comprising the same, and methods of using the same, such as in the prevention and/or treatment of diseases or conditions caused by influenza B viruses.

### CROSS-REFERENCE TO RELATED APPLICATIONS

This application claims priority to European application No. 23306481.5 filed 6 Sep. 2023, the entire contents of which are hereby incorporated by reference in their entirety.

### SUBMISSION OF SEQUENCE LISTING

The Sequence Listing associated with this application is filed in electronic format as a XML file and hereby incorporated by reference into the specification in its entirety. The name of the XML file containing the Sequence Listing is 0171.0085_SL.xlm and the size of the XML file is 17,440 bytes

### FIELD

This application relates to modified influenza B hemagglutinin polypeptides and nucleic acids, such as messenger ribonucleic acids (mRNAs), encoding the same, as well as compositions comprising the same, vaccines comprising the same, and methods of using the same, such as in the prevention and/or treatment of diseases or conditions caused by influenza B viruses.

### BACKGROUND

Influenza viruses cause substantial morbidity and mortality in humans across the globe, leading to the death of over half a million individuals annually. Before SARS-CoV-2 emerged, influenza was the leading cause of respiratory illness. Influenza B is estimated to account for approximately 25% of total influenza cases worldwide. Vaccination is the most common preventative measure utilized. Currently approved influenza vaccines are either inactivated vaccines, containing entire virions or virions subjected to treatment with agents that dissolve lipids (“split” vaccines), purified glycoproteins expressed in cell culture (“subunit vaccines”), or live attenuated virus vaccines, which are often produced in cell culture or eggs. Other types of vaccines, such as nucleic acid-based or viral vector-based vaccines, are also being developed. Since COVID-19, messenger RNAs (mRNA) have emerged as a new class of highly efficacious nucleic acids in the field of vaccines.

Influenza hemagglutinin (HA) is a prototypical class I fusion protein and a major component of current influenza vaccines. HA is a metastable trimeric glycoprotein and undergoes conformational changes from the so-called prefusion state to a postfusion state. However, the production of class I fusion proteins, like HA, by recombinant protein expression is challenging because of their general intrinsic instability, low expression levels, and failure to form correctly folded trimers. Recent data from clinical trials also suggest that mRNA vaccine encoding influenza B HA elicit sub-optimal neutralization response, which could impact its efficacy when used as a vaccine.

Accordingly, there is an urgent need to develop vaccines, either recombinant vaccine vectors or nucleic acid vaccines, that can deliver an influenza B HA with improved expression, quality, and stability.

### SUMMARY

Disclosed herein are modified influenza B hemagglutinin (HA) polypeptides having characteristics, such as increased immunogenic properties, improved stability in the prefusion conformation, improved expression, reduced sialic acid binding, and/or reduced antigenicity to non-neutralizing antibodies, that would enable elicitation of higher neutralization response that can translate into better vaccine efficacy, or lower reactogenicity when a lower dosage is use. Accordingly, in one aspect, provided herein is an artificial messenger ribonucleic acid (mRNA) encoding an influenza B HA polypeptide, wherein the influenza B HA polypeptide comprises: a) at least one proline substitution relative to a corresponding wild-type influenza B HA polypeptide, wherein the at least one proline substitution is at amino acid position 363, 366, 371, 372, 376, 380, 383, 390, 391, 393, 395, 397, 399, 421, 430, 431, 434, 436, and/or 490 as indexed by reference to the amino acid sequence of SEQ ID NO: 1; b) at least two cysteine substitutions relative to a corresponding wild-type influenza B HA polypeptide, wherein the at least two cysteine substitutions are at amino acid positions 20 and 387, 35 and 408, 36 and 415, 37 and 411, 125 and 431, 127 and 431, 185 and 223, 186 and 224, 186 and 239, 188 and 241, 232 and 433, 233 and 434, 239 and 276, 346 and 465, 367 and 478, 378 and 397, 380 and 397, 383 and 401, 387 and 510, 394 and 507, 394 and 510, 396 and 510, 396 and 514, 401 and 475, 430 and 437, 430 and 438, and/or 430 and 439, as indexed by reference to the amino acid sequence of SEQ ID NO: 1; c) at least one cavity filling amino acid substitution relative to a corresponding wild-type influenza B HA polypeptide, wherein the at least one cavity filling amino acid substitution is at amino acid position 460, 467, and/or 474 as indexed by reference to the amino acid sequence of SEQ ID NO: 1; d) one or more interface stabilization amino acid substitutions relative to a corresponding wild-type influenza B HA polypeptide, wherein the one or more interface stabilization amino acid substitutions are at amino acid position 18, 121, 188, 226, 228, 408, 435, and/or 460 as indexed by reference to the amino acid sequence of SEQ ID NO: 1; e) one or more pH sensor knock-out amino acid substitutions relative to a corresponding wild-type influenza B HA polypeptide, wherein the one or more pH sensor knock-out amino acid substitutions are at amino acid position 226, 228, 237, 239, 383, 388, 391, 401, 405, 408, 435, 460, 474 and/or 475 as indexed by reference to the amino acid sequence of SEQ ID NO: 1; f) at least one amino acid substitution relative to a corresponding wild-type influenza B HA polypeptide, wherein the at least one amino acid substitution generates or disrupts a N-linked glycosylation motif in the influenza B HA polypeptide and is at amino acid position 28, 60, 62, 141, 143, 186, 187, 214, 216, 223, 224, 336, and/or 349 as indexed by reference to the amino acid sequence of SEQ ID NO: 1; and/or g) at least one amino acid substitution relative to a corresponding wild-type influenza B HA polypeptide, wherein the at least one amino acid substitution is at amino acid position 157, 177, 218, and/or 257 as indexed by reference to the amino acid sequence of SEQ ID NO: 1.

In some embodiments, the influenza B HA polypeptide comprises two proline substitutions relative to a corresponding wild-type influenza B HA polypeptide, wherein the two proline substitutions are at amino acid positions 430 and 436 as indexed by reference to the amino acid sequence of SEQ ID NO: 1. In some embodiments, the influenza B HA polypeptide comprises five amino acid substitutions relative to a corresponding wild-type influenza B HA polypeptide, wherein the five amino acid substitutions are at amino acid positions 383, 401, 405, 408, and 475 as indexed by reference to the amino acid sequence of SEQ ID NO: 1. In some embodiments, the influenza B HA polypeptide comprises amino acid substitutions A430P and N436P as indexed by reference to the amino acid sequence of SEQ ID NO: 1. In some embodiments, the influenza B HA polypeptide comprises amino acid substitutions H383M, S401V, A405V, K408M, and H475M as indexed by reference to the amino acid sequence of SEQ ID NO: 1. In some embodiments, the influenza B HA polypeptide is from a B/Victoria influenza virus. In some embodiments, the B/Victoria influenza virus is B/Austria/1359417/2021. In some embodiments, the influenza B HA polypeptide comprises an amino acid sequence having at least about 90% sequence identity to the amino acid sequence of SEQ ID NO: 3 or SEQ ID NO: 5. In some embodiments, the influenza B HA polypeptide comprises the amino acid sequence of SEQ ID NO: 3 or SEQ ID NO: 5. In some embodiments, the artificial mRNA comprises a nucleic acid sequence having at least about 90% sequence identity to the nucleic acid sequence of SEQ ID NO: 4 or SEQ ID NO: 6. In some embodiments, the artificial mRNA comprises the nucleic acid sequence of SEQ ID NO: 4 or SEQ ID NO: 6.

In some embodiments, the artificial mRNA of the present disclosure comprises a 5′-cap structure and/or a 3′-poly(A) sequence. In some embodiments, the artificial mRNA comprises at least one chemically modified nucleotide and/or a phosphorothioate bond. In some embodiments, the at least one chemically modified nucleotide comprises a pseudouridine, a 2′-fluoro ribonucleotide, or a 2′-methoxy ribonucleotide, optionally wherein the pseudouridine is a N1-methylpseudouridine.

Also provided herein, in some embodiments, is a composition comprising the artificial mRNA of the present disclosure encapsulated in a lipid nanoparticle (LNP). In some embodiments, the LNP comprises a cationic lipid. In some embodiments, the cationic lipid comprises or is OF-02, cKK-E10, GL-HEPES-E3-E10-DS-3-E18-1, GL-HEPES-E3-E12-DS-4-E10, GL-HEPES-E3-E12-DS-3-E14, (4-hydroxybutyl)azanediyl]di(hexane-6,1-diyl) bis(2-hexyldecanoate) (ALC-0315), or IM-001. In some embodiments, the LNP further comprises a polyethylene glycol conjugated (PEGylated) lipid, a cholesterol-based lipid, and a helper lipid. In some embodiments, the PEGylated lipid comprises or is 1,2-dimyristoyl-rac-glycero-3-methoxypolyethylene glycol-2000 (DMG-PEG2000). In some embodiments, the cholesterol-based lipid comprises or is cholesterol. In some embodiments, the helper lipid comprises or is dioleoyl-sn-glycero-3-phosphoethanolamine (DOPE). the cationic lipid is present at a molar ratio between about 35% and about 55%, the PEGylated lipid is present at a molar ratio between about 0.25% and about 2.75%, the cholesterol-based lipid is present at a molar ratio between about 20% and about 45%, and the helper lipid is present at a molar ratio between about 5% and about 35%, wherein all of the molar ratios are relative to the total lipid content of the LNP. In some embodiments, the cationic lipid is present at a molar ratio of about 40%, the PEGylated lipid is present at a molar ratio of about 1.5%, the cholesterol-based lipid is present at a molar ratio of about 28.5%, and the helper lipid is present at a molar ratio of about 30%, wherein all of the molar ratios are relative to the total lipid content of the LNP. In some embodiments, the artificial mRNA encodes the influenza B HA polypeptide of SEQ ID NO: 3, and wherein the LNP comprises GL-HEPES-E3-E12-DS-4-E10 at a molar ratio of about 40%, DMG-PEG2000 at a molar ratio of about 1.5%, cholesterol at a molar ratio of about 28.5%, and DOPE at a molar ratio of about 30%. In some embodiments, the artificial mRNA encodes the influenza B HA polypeptide of SEQ ID NO: 5, and wherein the LNP comprises GL-HEPES-E3-E12-DS-4-E10 at a molar ratio of about 40%, DMG-PEG2000 at a molar ratio of about 1.5%, cholesterol at a molar ratio of about 28.5%, and DOPE at a molar ratio of about 30%. In some embodiments, the composition is an immunogenic composition.

In a further aspect, provided herein is an influenza B HA polypeptide comprising one or more amino acid substitutions relative to a corresponding wild-type influenza B HA polypeptide, wherein the one or more amino acid substitutions comprises: a) two proline substitutions at amino acid positions 430 and 436 as indexed by reference to the amino acid sequence of SEQ ID NO: 1; or b) amino acid substitutions at amino acid positions 383, 401, 405, 408, and 475 as indexed by reference to the amino acid sequence of SEQ ID NO: 1. In some embodiments, the influenza B HA polypeptide comprises amino acid substitutions A430P and N436P as indexed by reference to the amino acid sequence of SEQ ID NO: 1. In some embodiments, the influenza B HA polypeptide comprises amino acid substitutions H383M, S401V, A405V, K408M, and H475M as indexed by reference to the amino acid sequence of SEQ ID NO: 1. In some embodiments, the influenza B HA polypeptide is from a B/Victoria influenza virus. In some embodiments, the B/Victoria influenza virus is B/Austria/1359417/2021. In some embodiments, the influenza B HA polypeptide comprises an amino acid sequence having at least about 90% sequence identity to the amino acid sequence of SEQ ID NO: 3 or SEQ ID NO: 5. In some embodiments, the influenza B HA polypeptide comprises the amino acid sequence of SEQ ID NO: 3 or SEQ ID NO: 5.

Also provided herein, in some embodiments, is a trimeric influenza B HA polypeptide complex comprising three copies of any of the influenza B HA polypeptides disclosed herein. In some embodiments, the trimeric influenza B HA polypeptide complex of the present disclosure is more immunogenic as compared to a trimeric influenza B HA polypeptide complex prepared from a corresponding wild-type influenza B HA polypeptide without the amino acid substitutions. In some embodiments, the trimeric influenza B HA polypeptide complex of the present disclosure has a comparable immunogenicity as a trimeric influenza B HA polypeptide complex prepared from a corresponding wild-type influenza B HA polypeptide without the amino acid substitutions. In some embodiments, the immunogenicity is measured using a hemagglutination-inhibition assay.

Further provided is an artificial nucleic acid encoding any of the influenza B HA polypeptides disclosed herein. In some embodiments, the artificial nucleic acid comprises at least one chemically modified nucleotide and/or a phosphorothioate bond. In some embodiments, the disclosure provides a vector comprising the artificial nucleic acid disclosed herein. In some embodiments, the vector is a messenger RNA (mRNA) production vector. In some embodiments, the disclosure provides a host cell comprising the vector.

In a further aspect, the present disclosure provides a composition comprising any of the influenza B HA polypeptides disclosed herein, the trimeric influenza B HA polypeptide complexes, the artificial nucleic acids, or the vectors provided herein. In some embodiments, the composition is an immunogenic composition.

Also provided is a vaccine comprising any of the immunogenic compositions disclosed herein and a pharmaceutically acceptable carrier. In some embodiments, the vaccine further comprises an adjuvant. In some embodiments, the vaccine is an mRNA vaccine, and the vaccine further comprises an mRNA encoding an influenza H3 HA polypeptide and an mRNA encoding an influenza H1 HA polypeptide. In some embodiments, the vaccine is an mRNA vaccine, and the vaccine further comprises an mRNA encoding an influenza H3 HA polypeptide, an mRNA encoding an influenza H1 HA polypeptide, an mRNA encoding an influenza N2 neuraminidase (NA) polypeptide, an mRNA encoding an influenza N1 NA polypeptide, and an mRNA encoding an influenza NA polypeptide from an influenza B/Victoria lineage. In some embodiments, the vaccine is a recombinant vaccine, and the vaccine further comprises an influenza H3 HA polypeptide and an influenza H1 HA polypeptide. In some embodiments, the vaccine is a recombinant vaccine, and the vaccine further comprises an influenza H3 HA polypeptide, an influenza H1 HA polypeptide, an influenza N2 NA polypeptide, an influenza N1 NA polypeptide, and an influenza NA polypeptide from an influenza B/Victoria lineage.

Also provided herein, in another aspect, is a method of immunizing a subject, or a method of reducing one or more symptoms of an influenza B virus infection, the method comprising administering to the subject in need thereof any of the vaccines disclosed herein. In some embodiments, the disclosed method prevents an influenza B virus infection in the subject, decreases the subject's likelihood of getting an influenza B virus infection, or reduces the subject's likelihood of getting serious illness from an influenza B virus infection. In some embodiments, the subject is a human, such as one of 6 months of age or older, less than 18 years of age, at least 6 months of age and less than 18 years of age, at least 18 years of age and less than 65 years of age, at least 6 months of age and less than 5 years of age, at least 5 years of age and less than 65 years of age, at least 60 years of age, or at least 65 years of age. In some embodiments, the vaccine is administered intramuscularly, intradermally, subcutaneously, intravenously, intranasally, by inhalation, or intraperitoneally.

In a further aspect, provided herein is an in vitro method of preparing any of the trimeric influenza B HA polypeptide complexes disclosed herein, the method comprising culturing any of the host cells disclosed herein in a cell culture medium, and expressing the trimeric influenza B HA polypeptide complex. In some embodiments, the method further comprises a step of purifying the trimeric influenza B HA polypeptide complex from the cell culture medium.

### BRIEF DESCRIPTION OF THE DRAWING

The accompanying drawings, which are incorporated in and constitute a part of this specification, illustrate certain embodiments, and together with the written description, serve to explain certain principles of the methods and compositions disclosed herein.

FIG. 1 depicts the structure of an influenza B hemagglutinin (HA) trimer in the prefusion conformation. RBS: receptor binding site; FP: fusion peptide; RR1: refolding region 1; CH: central helix; RR2: refolding region 2.

FIG. 2A-2B depict representative modification designs focusing on pH sensor knockout and stem interface stabilization (FIG. 2A) and head interface stabilization (FIG. 2B). The numbering of the amino acid positions is as indexed by reference to the amino acid sequence of SEQ ID NO: 1.

FIG. 3 depicts an example gating scheme for flow cytometry data analyzing surface expressed HA on 293FT cells.

FIG. 4 depicts assessment of surface expression levels of representative modified influenza B/Austria/1359417/2021 HA polypeptides described in Example 1 on 293FT cells measured by flow cytometry using monoclonal antibodies CR8071 (vestigial esterase (VE) specific), R95-1D05 (receptor binding site (RBS) specific), and CR9114 (stem region-specific). Construct names are listed on the x-axis. 1_Aus: baseline control; 2_Aus and 5-Aus through 16_Aus: representative modified influenza B/Austria/1359417/2021 HA designs of the present disclosure. MFI: median fluorescence intensity.

FIG. 5 depicts assessment of surface expression levels of representative modified influenza B/Phuket/3073/2013 HA polypeptides described in Example 1 on 293FT cells measured by flow cytometry using monoclonal antibodies CR8071 (VE-specific), R95-1D05 (RBS-specific), and CR9114 (stem region-specific). Construct names are listed on the x-axis. 17_Phu: baseline control; 18_Phu and 21_Phu through 32_Phu: representative modified influenza B/Phuket/3073/2013 HA designs of the present disclosure. MFI: median fluorescence intensity.

FIG. 6 depicts exemplified substitutions mapped on prefusion influenza B HA (Protein Data Bank ID 4m44). Two of the trimeric HA are shown as ribbon diagram with another one shown as molecular surface. The side chains of stabilizing mutations are depicted as spheres and as sticks in zoomed views representing each stabilizing strategy. The numbering of the amino acid positions is as indexed by reference to the amino acid sequence of SEQ ID NO: 1.

FIG. 7 depicts assessment of surface expression levels of additional representative novel Austria HA polypeptides described in Example 2 on 293FT cells measured by flow cytometry using monoclonal antibodies CR8071 (VE-specific), R95-1D05 (RBS-specific), and CR9114 (stem region-specific). Construct names are listed on the x-axis. 1_Aus: baseline controls; 33_Aus through 43_Aus, 45_Aus through 52_Aus, and 54_Aus through 59_Aus: representative modified influenza B/Austria/1359417/2021 HA designs of the present disclosure. MFI: median fluorescence intensity.

FIG. 8 depicts assessment of surface expression levels of additional representative modified influenza B/Phuket/3073/2013 HA polypeptides described in Example 2 on 293FT cells measured by flow cytometry using monoclonal antibodies CR8071 (VE-specific), R95-1D05 (RBS-specific), and CR9114 (stem region-specific). Construct names are listed on the x-axis. 17_Phu: baseline control; 60_Phu through 70_Phu, 72_Phu through 79_Phu, and 81_Phu through 86_Phu: representative modified influenza B/Phuket/3073/2013 HA designs of the present disclosure. MFI: median fluorescence intensity.

FIG. 9 depicts assessment of surface expression levels of additional representative modified influenza B/Phuket/3073/2013 HA polypeptides described in Example 3 on 293FT cells measured by flow cytometry using monoclonal antibodies R95-1D05 (RBS-specific) and CR9114 (stem region-specific). Construct names are listed on the x-axis. Phu_17 or 17_Phu: baseline control. MFI: median fluorescence intensity.

FIG. 10 depicts assessment of surface expression levels of additional representative modified influenza B/Austria/1359417/2021 HA polypeptides described in Example 3 on 293FT cells measured by flow cytometry using monoclonal antibodies R95-1D05 (RBS-specific) and CR9114 (stem region-specific). Construct names are listed on the x-axis. 1_Aus: baseline control. MFI: median fluorescence intensity.

FIG. 11A-11D depict aggregated results obtained from Examples 1-3. FIG. 11A: Modified B/PHUKET/3073/2013 HAs having at least 2-fold increase in stem to RBS binding antibody ratio. FIG. 11B: Modified B/PHUKET/3073/2013 HA B/Austria/1359417/2021 HAs having at least 2-fold increase in stem to RBS binding antibody ratio. FIG. 11C: Common modification designs conferring at least 2-fold increase in stem to RBS binding antibody ratio in both B/PHUKET/3073/2013 and B/Austria/1359417/2021 strains. FIG. 11D: Aggregated results categorized by types of modification in each B/PHUKET/3073/2013 and B/Austria/1359417/2021 strain.

FIG. 12A-12B depict a modeled structure of an influenza B HA trimer showing the location of the amino acid substitutions in two representative modified influenza B HA polypeptides. FIG. 12A: construct 16_Aus containing H381M_H473M_S399V_K406M_A403V substitutions (left: prefusion conformation; right: postfusion conformation); FIG. 12B: construct 40_Aus containing A428P_N434P substitutions (left: prefusion conformation; right: postfusion conformation). The numbering of the amino acid positions is as indexed by reference to the amino acid sequence of SEQ ID NO: 2.

FIG. 13 depicts in vitro characterization of two representative modified influenza B HA polypeptides (“40_Aus” and “16_Aus”) as compared to the corresponding wild-type HA polypeptide (WT) using the percent total 293FT cells positive for monoclonal antibody binding measured by flow cytometry. The graphs depict the percentage of live cells that bound to each individual monoclonal antibody targeting HA from 30,000 cells (R95_1D05: RBD binding, CR9114: stem binding, and CR8071: VE binding). Construct names are listed on the x-axis and percent values of positive cells bound by each antibody on the y-axis.

FIG. 14 depicts the in vitro expression of two representative modified influenza B HA polypeptides (“40_Aus” and “16_Aus”) as compared to the corresponding wild-type HA polypeptide (WT) in mRNA-transfected HeLa cells.

FIG. 15 depicts fusogenicity (ability to mediate cell-cell fusion) of two representative modified influenza B HA polypeptides (“B/Austria_40” and “B/Aus 16”) as compared to the corresponding wild-type HA polypeptide (“B/Austria WT”) and a negative control (“B/Aus HA0”) which contains a substitution in the HA1-HA2 cleavage site and is fusion deficient.

FIG. 16 depicts the structural characterization of one representative modified influenza B polypeptide using negative stain electron microscopy (nsEM). Left: nsEM images of the wild-type HA polypeptide from B/Austria/1359417/2021 after low pH exposure; Right: nsEM images of the HA polypeptide from the representative modified influenza B polypeptide (40_Aus design) after low pH exposure.

FIG. 17A-17B depict the immunogenicity of representative modified influenza B HA polypeptides delivered as LNP-formulated mRNA in mice as compared to the wild-type HA polypeptide from B/Austria/1359417/2021. See Table 4 and Table 5 for the construct designs listed on the top of the graphs.

FIG. 18A-18B depict the immunogenicity of one representative modified influenza B HA polypeptide (40_Aus design) delivered as purified recombinant soluble protein as compared to the wild-type HA polypeptide from B/Austria/1359417/2021. FIG. 18A: HINT titer with cell-grown viruses; FIG. 18B: HA1 titer with egg-grown viruses.

FIG. 19A-19B depict in vitro expression of two representative modified influenza B HA polypeptide designs in a diverse panel of B/Victoria influenza virus strains. FIG. 19A: the 40_Aus design; FIG. 19B: the 16_Aus design.

FIGS. 20A and 20B depict in vitro characterization of the modified influenza B HA polypeptides generated based on a non-exhaustive combinatorial approach. See Table 8 for the construct designs listed on the top of the graphs. FIG. 20A: RBD-specific antibody (R95-1D05) binding and stem region-specific (CR9114) binding; FIG. 20B: ratio of stem region-specific (CR9114) binding over RBD-specific (R95-1D05) binding.

FIG. 21 depicts in vitro expression of the modified influenza B HA polypeptides generated based on a non-exhaustive combinatorial approach. See Table 8 for the construct designs listed on the top of the graphs.

### DETAILED DESCRIPTION

Reference will now be made in detail to various exemplary embodiments, examples of which are illustrated in the accompanying drawings and discussed in the detailed description that follows. It is to be understood that the following detailed description is provided to give the reader a fuller understanding of certain embodiments, features, and details of aspects of the disclosure, and should not be interpreted as limiting the scope of the disclosure.

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

### Definitions

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The term “about,” or “approximately,” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, 0.8%, 0.7%, ±0.6%, ±0.5%, ±0.4%, ±0.3%, ±0.2% or ±0.1% from the specified value as such variations are appropriate to perform the disclosed methods and/or to make and use the disclosed compositions. When “about” is present before a series of numbers or a range, it is understood that “about” can modify each of the numbers in the series or range.

An “amino acid” according to the present disclosure can be any of the twenty naturally occurring (or “standard” amino acids) or variants thereof, such as, for example, D-proline (the D-enantiomer of proline), or any variants that are not naturally found in proteins, such as norleucine. The standard amino acids can be divided into several groups based on their properties. Important factors are charge, hydrophilicity or hydrophobicity, size and functional groups. These properties are important for protein structure and protein-protein interactions. Some amino acids have special properties, such as cysteine that can form covalent disulfide bonds (or disulfide bridges) to other cysteine residues, proline that forms a cycle to the polypeptide backbone, and glycine that is more flexible than other amino acids. Table 1 shows the abbreviations and properties of the standard amino acids.

TABLE 1
Standard amino acids, abbreviations, and properties.

| | | | Side chain | Side chain | Side chain other |
|---|---|---|---|---|---|
| Amino acid | 3-Letter | 1-Letter | polarity | charge (pH 7.4) | properties |
| Alanine | Ala | A | Nonpolar | Neutral | Aliphatic, hydrophobic |
| Arginine | Arg | R | Polar | Positive | Basic |
| Asparagine | Asn | N | Polar | Neutral | Amidic |
| Aspartic acid | Asp | D | Polar | Negative | Acidic |
| Cysteine | Cys | C | Nonpolar | Neutral | Sulfur-containing |
| Glutamic acid | Glu | E | Polar | Negative | Acidic |
| Glutamine | Gln | Q | Polar | Neutral | Amidic |
| Glycine | Gly | G | Nonpolar | Neutral | Aliphatic |
| Histidine | His | H | Polar | Positive | Basic |
| | | | | (10%)/Neutral | |
| | | | | (90%) | |
| Isoleucine | Ile | I | Nonpolar | Neutral | Aliphatic, hydrophobic |
| Leucine | Leu | L | Nonpolar | Neutral | Aliphatic, hydrophobic |
| Lysine | Lys | K | Polar | Positive | Basic |
| Methionine | Met | M | Nonpolar | Neutral | Sulfur-containing, |
| | | | | | hydrophobic |
| Phenylalanine | Phe | F | Nonpolar | Neutral | Aromatic, hydrophobic |
| Proline | Pro | P | Nonpolar | Neutral | Aliphatic |
| Serine | Ser | S | Polar | Neutral | Hydroxylic |
| Threonine | Thr | T | Polar | Neutral | Hydroxylic |
| Typtophan | Trp | W | Nonpolar | Neutral | Aromatic, hydrophobic |
| Tyrosine | Tye | Y | Polar | Neutral | Aromatic, hydrophobic |
| Valine | Val | V | Nonpolar | Neutral | Aliphatic, hydrophobic |

The term “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein, the term “antibody” refers to an immunoglobulin molecule produced by B lymphoid cells with a specific amino acid sequence. In some embodiments, antibodies are evoked in humans or other animals by a specific antigen (immunogen). Antibodies are characterized by reacting specifically with the antigen in some demonstrable way, antibody and antigen each being defined in terms of the other. The terms “eliciting an antibody response,” “eliciting neutralizing antibody,” “eliciting immunogenic response,” or grammatical equivalents, refer to the ability of an antigen or other molecule to induce the production of antibodies. In some embodiments, the term “antibodies” refers to any recombinant antibodies used in in vitro assays, such as in HA screening assays, including one or more polypeptides substantially encoded by immunoglobulin genes or fragments of immunoglobulin genes. Such antibodies may exist as intact immunoglobulins or as fragments of the immunoglobulin classes, IgG, IgM, IgA, IgD and IgE, respectively. Exemplary antibody fragments include, but are not limited to, F(ab)′2, Fab′, and single chain Fv (scFv).

As used herein, the term “antigen” refers to an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, ferrets, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, including all values and subranges therebetween, of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity, including those induced by heterologous immunogens. Antigens include the modified influenza B HA polypeptides described herein.

As used herein, an “artificial nucleic acid molecule” or the like may typically be understood to be a nucleic acid, e.g., a DNA or an RNA, that does not occur naturally. Thus, an “artificial messenger ribonucleic acid (mRNA)” refers to an mRNA that does not occur naturally. In other words, an artificial nucleic acid molecule may be understood as a non-natural nucleic acid molecule or rRNA molecule. Such nucleic acid or rRNA molecule may be non-natural due to its individual sequence (which does not occur naturally) and/or due to other modifications, e.g., structural modifications of nucleotides that do not occur naturally. An artificial nucleic acid molecule may be a DNA molecule, an RNA molecule (e.g., mRNA), or a hybrid molecule comprising DNA and RNA portions. Typically, artificial nucleic acid molecules may be designed and/or generated by genetic engineering methods to correspond to a desired artificial sequence of nucleotides (heterologous sequence). Further, the term “artificial nucleic acid molecule” or the like (e.g., “artificial nRNA”) is not restricted to mean “one single molecule” but is, typically, understood to comprise an ensemble of identical molecules. Accordingly, it may relate to a plurality of identical molecules contained in an aliquot.

The phrase “as indexed by reference to the amino acid sequence of SEQ ID NO: 1,” as used herein, refers to a normalized biological sequence alignment that allows the comparison of a query sequence (e.g., a modified HA polypeptide sequence to which one or more of the modifications described herein have been or will be applied) to a subject sequence (e.g., a wild-type influenza HA polypeptide sequence, such as the HA polypeptide sequence of B/Phuket/3073/2013 (SEQ ID NO: 1)), thereby identifying amino acid residues in the target sequence that correspond to the same positions in the subject sequence. In general, the target sequence and the query sequence share characteristic portions or features but differ slightly in length and/or sequence identity. For example, the numbering of residues in a specific target sequence or for targeted modification can be identified and described based on the B/Phuket/3073/2013 amino acid sequence. Sequences are aligned to the full-length HA protein sequence (including signal peptide, transmembrane and cytoplasmic tail domains) of B/Phuket/3073/2013 (SEQ ID NO: 1). The N-terminal methionine of the signal peptide is residue 1. Accordingly, the phrase “amino acid position x as indexed by reference to the amino acid sequence of SEQ ID NO: 1” is used herein to designate the position/identity of an amino acid residue in a polypeptide of interest (e.g., a modified influenza B HA polypeptide) by referring to the corresponding amino acid at position x in the HA polypeptide sequence of B/Phuket/3073/2013 (SEQ ID NO: 1). Similarly, when referring to SEQ ID NO: 2, the phrase “as indexed by reference to the amino acid sequence of SEQ ID NO: 2” is used herein to designate the position/identity of an amino acid residue in a polypeptide of interest (e.g., a modified influenza B HA polypeptide) by referring to the corresponding amino acid position in the HA polypeptide sequence of B/Austria/1359417/2021 (SEQ ID NO: 2).

The term “at least,” “less than,” “more than,” or “up to” prior to a number or series of numbers (e.g., “at least two”) is understood to include the number adjacent to the term “at least,” “less than” or “more than,” and all subsequent numbers or integers that could logically be included, as clear from context. When the term “at least,” “less than,” “more than,” or “up to” is present before a series of numbers or a range, it is understood that “at least,” “less than,” “more than,” or “up to” can modify each of the numbers in the series or range.

The term “biological activity,” as used herein, refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest. In some embodiments, the biological activity of an HA polypeptide refers to the ability of the HA polypeptide to elicit neutralizing antibody. In these cases, the term “biological activity” is used interchangeably with “immunogenic activity.”

As used herein, a “codon-optimized” nucleic acid sequence refers to a nucleic acid sequence that has been altered such that expression of the encoded protein is improved and optimized for a particular expression system. A “codon-optimized” nucleic acid sequence encodes the same protein as a non-optimized parental sequence upon which the “codon-optimized” nucleic acid sequence is based. For example, a nucleic acid sequence may be “codon-optimized” for expression in mammalian cells (e.g., CHO cells, human cells, mouse cells etc.), bacterial cells (e.g., E. coli), insect cells, yeast cells or plant cells.

As used herein, the term “epitope” includes any moiety that is specifically recognized by an immunoglobulin (e.g., antibody or receptor) binding component in whole or in part. In some embodiments, an epitope is comprised of a plurality of amino acid residues in an antigen. In some embodiments, the amino acid residues are surface-exposed when the antigen adopts a relevant three-dimensional conformation. In some embodiments, the amino acid residues are physically near to or continuous with each other in space when the antigen adopts such a conformation. In some embodiments, at least some of the amino acids are physically separated from one another when the antigen adopts an alternative conformation (e.g., is linearized; e.g., a non-linear epitope).

As used herein, the term “head region” refers to a segment of an influenza B HA polypeptide that is encompassed by approximately amino acid residues 57-307 as indexed by reference to the amino acid sequence of SEQ ID NO: 1. Morphologically, the head region may be defined as the globular shaped domain of HA.

As used herein, the term “hemagglutinin polypeptide” or “HA polypeptide” refers to a polypeptide whose amino acid sequence includes at least one characteristic sequence of an influenza type A or type B HA. A wide variety of HA sequences from influenza isolates are known in the art; indeed, the National Center for Biotechnology Information (NCBI) maintains a database (ncbi.nlm.nih.gov/genomes/FLU/) that includes more than 40,000 HA sequences (for type A and B viruses). Those of ordinary skill in the art, referring to this database, can readily identify sequences that are characteristic of HA polypeptides generally, and/or of particular HA polypeptides (e.g., influenza type B HAs or influenza type A HAs, such as H1, H2, H3, H4, H5, H6, H7, H8, H9, H10, H11, H12, H13, H14, H15, or H16 polypeptides; or of HAs that mediate infection of particular hosts, e.g., human, avian, seal etc.).

As used herein, “H1” refers to an influenza virus subtype 1 hemagglutinin (HA). Type A influenza viruses are divided into Groups 1 and 2. Groups 1 and 2 are further divided into subtypes, which refers to classification of a virus based on the sequences of two proteins on the surface of the virus HA and neuraminidase (NA). Currently, there are 18 recognized HA subtypes (H1-H18). H1 is thus distinct from the other HA subtypes, including H2-H18.

As used herein, “H3” refers to an influenza virus subtype 3 HA. H3 is thus distinct from the other HA subtypes, including H1, H2 and H4-H18.

As used herein, “N1” refers to an influenza virus subtype 1 NA. Type A influenza viruses are divided into Groups 1 and 2. Groups 1 and 2 are further divided into subtypes, which refers to classification of a virus based on the sequences of two proteins on the surface of the virus HA and NA. Currently, there are 11 recognized NA subtypes (N1-N11). N1 is thus distinct from the other NA subtypes, including N2-N11.

As used herein, “N2” refers to an influenza virus subtype 2 neuraminidase (NA). N2 is thus distinct from the other NA subtypes, including N1 and N3-N11.

The term “host” is used herein to refer to a system (e.g., a cell, organism, etc.) in which a polypeptide of interest is present. In some embodiments, a host is a system that is susceptible to infection with a particular infectious agent. In some embodiments, a host is a system that expresses a particular polypeptide of interest.

As used herein, the term “host cell” refers to a cell into which exogenous DNA (recombinant or otherwise) has been introduced. For example, host cells may be used to produce the modified influenza B HA polypeptides described herein by standard recombinant techniques. Persons of skill upon reading this disclosure will understand that such terms refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell: as used herein. In some embodiments, host cells include any prokaryotic and eukaryotic cells suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, microalgae (including both eukaryotic algae, such as Chlamydomonas, Chlorella, Nannochloropsis, Thraustochytriales (e.g., Schizochytrium sp.), diatoms (e.g., Phaeodactylum), and prokaryotic cyanobacteria, also known as blue-green algae such as Arthrospira), insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-11 CHO, Veggie-CHO), COS (e.g., COS-7), retinal cell, Vero, CV1, kidney (e.g., HEK293, 293 EBNA, MSR 293, MDCK, Ha K, 131K), HeLa, HepG2, W138, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell. In some embodiments, the cell comprises one or more viral genes, e.g., a retinal cell that expresses a viral gene (e.g., a PER.C6™ cell).

As used herein, the term “in some embodiments,” “in certain embodiments,” “in other embodiments,” “in some other embodiments,” or the like, refers to embodiments of all aspects of the disclosure, unless the context clearly indicates otherwise.

As used here, an “mRNA vaccine” refers to a type of vaccine that uses messenger RNA (mRNA) to produce an immune response.

The term “mutation” refers to deletion, addition, or substitution of an amino acid residue in the amino acid sequence of a modified protein or polypeptide as compared to the amino acid sequence of a reference protein or polypeptide.

As used herein, the term “N-linked glycosylation motif” refers to an amino acid sequence on the surface of a polypeptide, such as a protein, which accommodates the attachment of a glycan. An N-linked glycosylation motif comprises a consensus sequence of NxS/Ty, in which N is asparagine, x and y are any residue except proline (P), and S/T is a serine or threonine residue. A glycan is a polysaccharide or oligosaccharide. Glycan may also be used to refer to the carbohydrate portion of a glycoconjugate, such as a glycoprotein, glycolipid, or a proteoglycan.

The term “prevent,” “preventing,” or “prevention,” as used herein, refers to prophylaxis, avoidance of disease manifestation, a delay of onset, and/or reduction in frequency and/or severity of one or more symptoms of a particular disease, disorder or condition (e.g., infection with, for example, a virus, such as influenza virus). In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

As used herein, the term “prophylactically effective amount” means an amount sufficient to avoid disease manifestation, delay onset of and/or reduce in frequency and/or severity one or more symptoms of a particular disease, disorder or condition (e.g., infection with, for example, a virus, such as influenza virus).

The term “sequence identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Sequence identity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). Typical methods to determine identity are designed to give the largest match between the sequences tested. Methods to determine sequence identity and similarity are codified in publicly available computer programs. Typical computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity. In some embodiments, the sequence identity is determined using the BLAST program with the default parameters.

As used herein, the term “receptor-binding site” or “RBS” comprises contiguous or non-contiguous amino acid residues of the head region of an influenza HA polypeptide, which include amino acids involved in direct binding of sialic acid on the target cell receptor proteins. Amino acid residues that make up a “receptor-binding site” or “RBS” of an influenza HA polypeptide may be described from crystal structures of HA polypeptides complexed with sialic acid analogs and identifying amino acid residues within a certain proximity to the analog or may be described in reference to an HA polypeptide sequence from a particular viral strain (e.g., B/Victoria/02/1987, B/Yamagata/16/1988). Thus, in some embodiments, the “receptor-binding site” or “RBS” of a modified HA polypeptide as described herein may be determined using a reference HA polypeptide sequence. In some embodiments, the “receptor-binding site” or “RBS” of a modified HA polypeptide as described herein may be determined using the crystal structures of HA polypeptide sequence. An exemplary reference crystal structure of HA polypeptide includes that of influenza virus B/Yamanashi/166/1998 in complex with avian-like receptor LSTa (PDB ID 4M40). Accordingly, in some embodiments, the RBS may be defined as an area including all amino acid residues within 5 angstroms of the LSTa molecule in the crystal structure of HA of influenza virus B/Yamanashi/166/1998 in complex with LSTa (PDB ID 4M40). In some embodiments, the RBS may be defined as a region of the modified influenza B HA polypeptide made up of residues at amino acid positions 110, 151-157, 165, 173-175, 208-218, 248, and 254-259 as indexed by reference to the amino acid sequence of SEQ ID NO: 1.

As used herein, a “recombinant vaccine” refers to a type of vaccine that uses genetic engineering to produce antigens from a pathogen (e.g., influenza virus) using a harmless organism, such as yeast or bacteria.

As used herein, the term “stem region” refers to a discontinuous region of an influenza B HA polypeptide, the region comprising approximately amino acid residues 16-56 and 308-547 as indexed by reference to the amino acid sequence of SEQ ID NO: 1. Morphologically, the stem region may be defined as the elongated domain that emerges from the globular head.

As used herein, the term “subject” means any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In some embodiments, the non-human subject is a mammal (e.g., a rodent, a mouse, a rat, a rabbit, a ferret, a monkey, a dog, a cat, a sheep, cattle, a primate, and/or a pig). In some embodiments, a subject may be a transgenic animal, genetically-engineered animal, and/or a clone. In some embodiments, the subject is an adult, an adolescent or an infant. In some embodiments, the term “individual” or “patient” is used and is intended to be interchangeable with the term “subject.”

As used herein, the term “vector” refers to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

As used herein, the term “wild-type” generally refers to a normal form of a protein or nucleic acid, as is found in nature. For example, wild-type HA polypeptides are found in natural isolates of influenza virus. A variety of different wild-type HA sequences can be found in the NCBI influenza virus sequence database (ncbi.nlm.nih.gov/genomes/FLU/).

### Structure of Influenza B HA Polypeptides

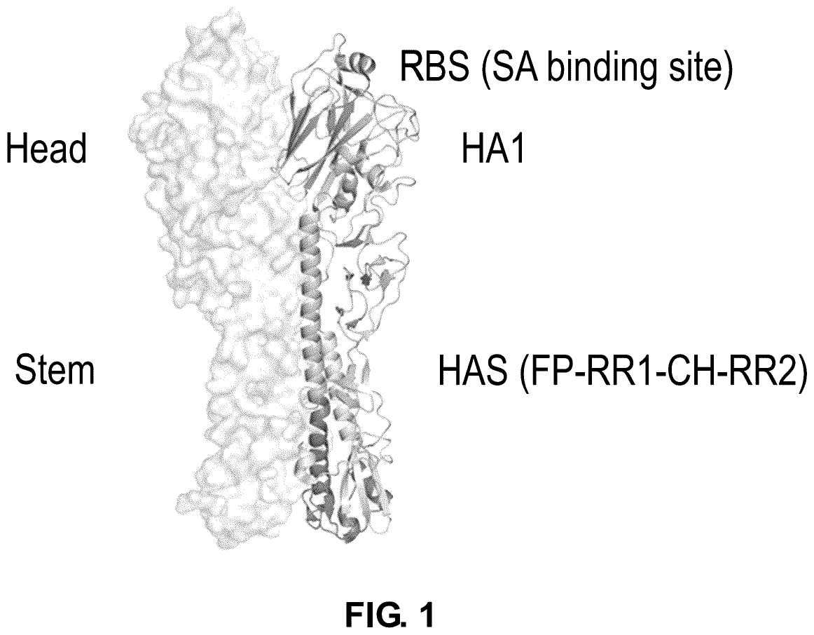

Influenza B HA is a trimeric glycoprotein that is anchored to the viral coat and has two main functions. During the entry process, HA first mediates attachment of the virus to the surface of target cells through interactions with sialic acid receptors and then, after endocytosis of the virus, it triggers the fusion of the viral and endosomal membranes to release its genome into the cytoplasm of the target cell. Influenza B HA comprises a large ectodomain of about 500 amino acids that is cleaved by host-derived enzymes to generate 2 polypeptides that remain linked by a disulfide bond. The majority of the N-terminal fragment (about 320-330 amino acids), known as HA1, forms a membrane-distal globular domain, also called the head region, that contains the receptor-binding site (RBS) and most determinants recognized by virus-neutralizing antibodies. The smaller C-terminal portion (about 180 amino acids), known as HA2, consisting of a fusion peptide, a N-terminal refolding region 1 (RR1), a central helix, and a C-terminal refolding region 2 (RR2), forms a stem-like structure, also called the stem region, that anchors the head region to the cellular or viral membrane. Three monomeric HA subunits combine to form the trimeric biological HA molecule through symmetry operations. A schematic structure of an influenza B HA trimer is shown in FIG. 1.

Like other class I fusion proteins, influenza B HA transforms from a high-energy, metastable prefusion state to a postfusion conformation, a transition triggered by low pH. Without intending to be bound by any theory, the pH-based triggering of HA conformational changes is generally thought to be due to the protonation of amino acids that act as pH sensors. However, the residue or combination of residues acting as pH sensors has not yet been fully elucidated. Since the membrane fusion event occurs in the pH range of about 5-6, the most likely residues to function as pH sensors are histidines, aspartates and/or glutamates, which possess a pKₐ in the appropriate pH range.

Most HA neutralizing antibodies bind to the loops that surround the RBS and interfere with receptor binding and attachment. Since these loops are highly variable, most antibodies targeting these regions are strain specific. Functional and structural analysis of recently developed fully human monoclonal antibodies against influenza A HA with broad cross-neutralizing potency revealed that, rather than targeting the receptor binding and attachment, these antibodies interfere with the membrane fusion process and are directed against highly conserved epitopes in the stem domain of the influenza A HA protein. See, Throsby et al., PLoS One, 2008, 3(12):e3942, Ekiert et al., Science, 2009, 324(5924):246-251, WO 2008/028946, WO2010/130636, and WO 2013/007770, all incorporated herein by reference.

The amino acid sequences of a large number of influenza B HA polypeptides from different influenza B viruses of both B/Yamagata lineage and B/Victoria lineage, as well as nucleic acid sequences encoding such polypeptides, are known in the art and readily available in, for instance, the Influenza Virus Database maintained by the National Center for Biotechnology Information (NCBI) (ncbi.nlm.nih.gov/genomes/FLU/). By way of example, the amino acid sequences of wild-type HA polypeptides from a representative influenza B virus of the B/Yamagata lineage and a representative influenza B virus of the B/Victoria lineage are set forth in SEQ ID NO: 1 and SEQ ID NO: 2, respectively.

HA polypeptide of influenza B/Phuket/3073/2013
(a B/Yamagata Lineage):
(SEQ ID NO: 1)
MKAIIVLLMVVTSNADRICTGITSSNSPHVVKTATQGEVNVTGVIPLT
TTPTKSYFANLKGTRTRGKLCPDCLNCTDLDVALGRPMCVGTTPSAKA
SILHEVRPVTSGCFPIMHDRTKIRQLPNLLRGYEKIRLSTQNVIDAEK
APGGPYRLGTSGSCPNATSKIGFFATMAWAVPKDNYKNATNPLTVEVP
YICTEGEDQITVWGFHSDNKTQMKSLYGDSNPQKFTSSANGVTTHYVS
QIGDFPDQTEDGGLPQSGRIVVDYMMQKPGKTGTIVYQRGVLLPQKVW
CASGRSKVIKGSLPLIGEADCLHEEYGGLNKSKPYYTGKHAKAIGNCP
IWVKTPLKLANGTKYRPPAKLLKERGFFGAIAGFLEGGWEGMIAGWHG
YTSHGAHGVAVAADLKSTQEAINKITKNLNSLSELEVKNLQRLSGAMD
ELHNEILELDEKVDDLRADTISSQIELAVLLSNEGIINSEDEHLLALE
RKLKKMLGPSAVDIGNGCFETKHKCNQTCLDRIAAGTFNAGEFSLPTF
DSLNITAASLNDDGLDNHTILLYYSTAASSLAVTLMLAIFIVYMVSRD
NVSCSICL.
HA polypeptide of influenza B/Austria/1359417/
2021 (a B/Victoria lineage):
(SEQ ID NO: 2)
MKAIIVLLMVVTSNADRICTGITSSNSPHVVKTATQGEVNVTGVIPLT
TTPTKSHFANLKGTETRGKLCPKCLNCTDLDVALGRPKCTGKIPSARV
SILHEVRPVTSGCFPIMHDRTKIRQLPNLLRGYEHVRLSTHNVINTED
APGGPYEIGTSGSCLNITNGKGFFATMAWAVPKNKTATNPLTIEVPYI
CTEEEDQITVWGFHSDDETQMARLYGDSKPQKFTSSANGVTTHYVSQI
GGFPNQTEDGGLPQSGRIVVDYMVQKSGKTGTITYQRGILLPQKVWCA
SGKSKVIKGSLPLIGEADCLHEKYGGLNKSKPYYTGEHAKAIGNCPIW
VKTPLKLANGTKYRPPAKLLKERGFFGAIAGFLEGGWEGMIAGWHGYT
SHGAHGVAVAADLKSTQEAINKITKNLNSLSELEVKNLQRLSGAMDEL
HNEILELDEKVDDLRADTISSQIELAVLLSNEGIINSEDEHLLALERK
LKKMLGPSAVEIGNGCFETKHKCNQTCLDRIAAGTFDAGEFSLPTFDS
LNITAASLNDDGLDNHTILLYYSTAASSLAVTLMIAIFVVYMVSRDNV
SCSICL.

The HA polypeptides of influenza B viruses exhibit remarkable sequence conservation across the two existing lineages. For example, the HA polypeptide of B/Phuket/3073/2013, a B/Yamagata Lineage (SEQ ID NO: 1), and the HA polypeptide of B/Austria/1359417/2021, a B/Victoria lineage (SEQ ID NO: 2), share 92% sequence identity. Within the same lineage, the sequence identity is even greater. In view of the substantial conservation of the influenza B HA sequences, a person of ordinary skill in the art can easily compare amino acid positions between different influenza B HA sequences to identify corresponding amino acid positions between different influenza B virus strains.

Thus, for the purposes of the present disclosure (unless context indicates otherwise), the amino acid positions in the modified influenza B HA polypeptides are given with reference to the amino acid sequence of the full length wild-type HA polypeptide of B/Phuket/3073/2013 set forth in SEQ ID NO: 1. However, it should be noted, and one of skill in the art will understand, that different influenza B HA sequences may have different numbering systems, for example, if there are additional amino acid residues added or removed as compared to SEQ ID NO: 1. As such, it is to be understood that when specific amino acid residues are referred to by their number, the description is not limited to only amino acids located at precisely that numbered position when counting from the beginning of a given amino acid sequence, but rather that the equivalent/corresponding amino acid residue in any and all influenza B HA polypeptide sequences is intended even if that residue is not at the same precise numbered position, for example if a given influenza B HA polypeptide sequence is shorter or longer than SEQ ID NO: 1, or has insertions or deletions as compared to SEQ ID NO: 1.

### Modified Influenza B HA Polypeptides

The present disclosure provides modified influenza B HA polypeptides in which one or more mutations have been introduced in the amino acid sequence relative to the amino acid sequence of the corresponding wild-type influenza B HA polypeptide. In some embodiments, the modified influenza B HA polypeptides disclosed herein possess certain beneficial characteristics, such as increased immunogenic properties, improved stability in the prefusion conformation, improved expression, reduced sialic acid binding, and/or reduced antigenicity to non-neutralizing antibodies, as compared to the corresponding wild-type influenza B HA polypeptide. In some embodiments, the modified influenza B HA polypeptides disclosed herein present epitopes for recognition by broadly protecting antibodies and thus, are useful for creating a universal epitope-based vaccine for inducing protection against a broad range of influenza B virus strains. Also provided are artificial nucleic acid molecules that encode the modified influenza B HA polypeptides disclosed herein.

The modified influenza B HA polypeptides of the present disclosure comprise one or more amino acid mutations in the head region and/or the stem region as compared to the amino acid sequence of the corresponding wild-type influenza B HA polypeptide. The introduced amino acid mutations in the modified influenza B HA polypeptides of the disclosure include amino acid substitutions, deletions, or additions. In some embodiments, the one or more amino acid mutations may be independently selected from substitutions, insertions, deletions, and truncations. In some embodiments, the only mutations introduced in the amino acid sequence of the modified influenza B HA polypeptides of the disclosure are amino acid substitutions relative to the corresponding wild-type influenza B HA polypeptide, and may include conservative and/or non-conservative substitutions.

Conservative substitutions may be made, for instance, on the basis of similarity in polarity, charge, size, solubility, hydrophobicity, hydrophilicity, and/or the amphipathic nature of the amino acid residues involved. For example, as summarized in Table 1, the 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe. As used herein, “conservative substitutions” are defined as exchanges of an amino acid by another amino acid listed within the same group of the six standard amino acid groups shown above. For example, the exchange of Asp by Glu retains one negative charge in the modified polypeptide. In addition, glycine and proline may be substituted for one another based on their ability to disrupt α-helices. As used herein, “non-conservative substitutions” are defined as exchanges of an amino acid by another amino acid listed in a different group of the six standard amino acid groups shown above.

In some embodiments, the substitutions may also include non-classical amino acids (e.g. selenocysteine, pyrrolysine, N-formylmethionine β-alanine, GABA and 6-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, T-Abu, F-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as R methyl amino acids, C α-methyl amino acids, N α-methyl amino acids, and amino acid analogs in general). In some embodiments, amino acid substitutions at a specific amino acid position are chosen based on factors which include, but are not limited to, potential for steric hindrance, charge attraction, charge repulsion, common properties of the amino acid side chain, secondary and/or tertiary structure considerations, and/or frequency of use in respective host cells. A person skilled in the art would understand which factors to consider when designing amino acid substitutions for the modified influenza B HA polypeptides of the present disclosure.

Accordingly, provided herein is a modified influenza B HA polypeptide comprising a head region and a stem region, wherein the modified influenza B HA polypeptide comprises one or more modifications selected from proline mutations, disulfide bridge forming mutations, interface stabilizing mutations, pH sensor inactivating mutations, glycan engineering mutations in the head region, glycan engineering mutations in the stem region, and/or sialic acid binding interfering mutations as described in detail herein below. In some embodiments, the modified influenza B HA polypeptides of the disclosure comprise at least one of two of the aforementioned modifications, such as at least one proline mutation and at least one disulfide bridge forming mutation, or at least one disulfide bridge forming mutation and at least one pH sensor inactivating mutation. In some embodiments, the modified influenza B HA polypeptides of the disclosure comprise at least one of three of the aforementioned modifications, such as at least one proline mutation, at least one disulfide bridge forming mutation, and at least one pH sensor inactivating mutation. In some embodiments, the modified influenza B HA polypeptides of the disclosure comprise at least one of four of the aforementioned modifications, such as at least one proline mutation, at least one disulfide bridge forming mutation, at least one interface stabilizing mutation, and at least one pH sensor inactivating mutation. In some embodiments, the modified influenza B HA polypeptides of the disclosure comprise at least one of five of the aforementioned modifications, such as at least one proline mutation, at least one disulfide bridge forming mutation, at least one interface stabilizing mutation, at least one pH sensor inactivating mutation, and at least one glycan engineering mutation in the head region. In some embodiments, the modified influenza B HA polypeptides of the disclosure comprise at least one of six of the aforementioned modifications, such as at least one proline mutation, at least one disulfide bridge forming mutation, at least one interface stabilizing mutation, at least one pH sensor inactivating mutation, at least one glycan engineering mutation in the head region, and at least one sialic acid binding interfering mutations. In some embodiments, the modified influenza B HA polypeptides of the disclosure comprise at least one of each of the seven aforementioned modifications.

In some embodiments, the one or more aforementioned modifications stabilize the modified influenza B HA polypeptides of the disclosure in a prefusion conformation. Monoclonal antibodies to well-defined, structural epitopes of the influenza B HA polypeptide, such as stem region-specific antibodies (e.g., CR9114), vestigial esterase (VE) specific antibodies (e.g., CR8071), and receptor binding site (RBS) specific antibodies (e.g., R95-1D05), can be used to investigate the conformation of the modified influenza B HA polypeptides disclosed herein using flow cytometry. The binding of an RBS-specific antibody, such as R95-1D05, indicates the total surface expression and integrity of sialic acid binding pocket on the surface expressed HA polypeptides. The binding of a VE-specific antibody, such as CR8071, and a stem-region-specific antibody, such as CR9114, indicates that the surface expressed HA polypeptide is in a prefusion conformation. The binding ratio of a stem-region-specific antibody, such as CR9114, to an RBS-specific antibody, such as R95-1D05 (e.g., CR9114 MFI/R95-1D05 MFI) indicates the ratio of prefusion, closed HA polypeptides to total HA polypeptides presented on the cell surface, with a higher ratio indicating that a higher percentage of the surface expressed HA polypeptides is in a prefusion, locked conformation. Accordingly, in some embodiments, stabilization of the prefusion conformation is measured by an increased binding of the modified influenza B HA polypeptide to a stem region-specific antibody (e.g., CR9114) as compared to the corresponding wild-type influenza B HA polypeptide. In certain embodiments, stabilization of the prefusion conformation is measured by an increased (e.g., at least two-fold higher) binding ratio of a stem region-specific antibody (e.g., CR9114) to an RBS-specific antibody (e.g., R95-1D05) as compared to the corresponding wild-type influenza B HA polypeptide. In some embodiments, stabilization of the prefusion conformation is measured by an increased binding of the modified influenza B HA polypeptide to a stem region-specific antibody (e.g., CR9114) and an increased binding ratio (e.g., two or greater) of a stem region-specific antibody (e.g., CR9114) to an RBS-specific antibody (e.g., R95-1D05) as compared to the corresponding wild-type influenza B HA polypeptide.

Morphologically, the head region may be defined as the globular shaped domain of HA and the stem region may be defined as the elongated domain that emerges from the globular head. A schematic structure of the modified influenza B HA polypeptides of the disclosure is provided in FIG. 1. In some embodiments, the head region of a modified influenza B HA polypeptide according to the present disclosure can be defined as being a segment of the modified influenza B HA polypeptide at approximately amino acid positions 57-307 as indexed by reference to the amino acid sequence of SEQ ID NO: 1. In some embodiments, the stem region, being a discontinuous region of the HA polypeptide, can be defined as comprising approximately amino acid residues 16-56 and 308-547 of the modified influenza B HA polypeptide as indexed by reference to the amino acid sequence of SEQ ID NO: 1.

Wild-type influenza B HA polypeptides comprise a signal peptide at their N-terminus (e.g., corresponding to amino acids 1-15 of SEQ ID NO: 1), which directs transport of the HA polypeptide during production and generally is not present in the final polypeptide that is, for instance, used in a vaccine. Accordingly, in some embodiments, the modified influenza B HA polypeptides of the present disclosure do not comprise a signal peptide. In other embodiments, the modified influenza B HA polypeptides of the present disclosure comprise a signal peptide that is native to the wild-type influenza B HA polypeptide to which the one or more modifications are introduced. In some embodiments, the modified influenza B HA polypeptides of the present disclosure comprise a signal peptide that is heterologous to the wild-type influenza B HA polypeptide to which the one or more modifications are introduced. In some embodiments, such a heterologous signal peptide is an influenza HA signal peptide derived from an influenza A HA polypeptide. In some embodiments, such a heterologous signal peptide is an influenza HA signal peptide derived from an influenza B HA polypeptide that is different from the wild-type influenza B HA polypeptide to which the one or more modifications are introduced. In some embodiments, the heterologous signal peptide is from a non-influenza source. Any signal peptide known in the art that can direct transport of the HA polypeptide during production can be used. For recombinant production of the modified influenza B HA polypeptides of the disclosure in insect cells, for instance, any signal peptides from both mammals and viruses can be used to guide protein secretion in insect cells.

The influenza B HA polypeptides to which the one or more modifications according to the present disclosure are introduced can be from any wild-type influenza B viruses known in the art or discovered in the future, including, but not limited to, the HA polypeptide of a B/Yamagata influenza virus, such as B/Phuket/3073/2013, and the HA polypeptide of a B/Victoria influenza virus, such as B/Austria/1359417/2021. Additional B/Victoria influenza viruses may include, but are not limited to, B/Washington/02/2019, B/Lisboa/37/2019, B/India/Punniv564/2021, B/Hunanjishou/1678/2021, B/Yekaterinburg/3292V/2020, B/Kenya/180/2021, B/Qinghaigonghe/1259/2021, B/Shanghaihuangpu/1825/2021, B/Bangladesh/2002/2019, B/Colorado/06/2017, and B/Brisbane/60/2008. In some embodiments, the modified influenza B HA polypeptide of the disclosure is from a B/Yamagata influenza virus. In some embodiments, the modified influenza B HA polypeptide of the disclosure is from the influenza B/Phuket/3073/2013 strain. In some embodiments, the modified influenza B HA polypeptide of the disclosure is from a B/Victoria influenza virus. In some embodiments, the modified influenza B HA polypeptide of the disclosure is from the influenza B/Austria/1359417/2021 strain. In certain embodiments, the modified influenza B HA polypeptide of the disclosure is from a standard of care strain. It should be understood that any influenza B strain not specifically mentioned herein can be the source of the HA polypeptide to which the one or more modifications according to the present disclosure are introduced.

### Proline Mutations

In some embodiments, relative to a corresponding wild-type influenza B HA polypeptide, the modified influenza B HA polypeptides provided herein comprise at least one proline mutation (e.g., substitution) in the stem region (comprising amino acid residues 16-56 and 308-547 as indexed by reference to the amino acid sequence of SEQ ID NO: 1). The introduction of the at least one proline mutation is designed to break at least one helical structure, such as an α-helix, that appears in the stem region of an influenza B HA polypeptide in a postfusion conformation. Without wishing to be bound by any theory, proline is the only naturally occurring amino acid in which the side chain is bonded to the backbone nitrogen, forming a five-membered pyrrolidine ring. This pyrrolidine ring restricts the rotation of the N-Cα bond, decreasing the backbone conformational entropy of the unfolded form of the protein relative to other naturally occurring amino acids. Thus, without intending to be bound by any theory, introducing a proline mutation may increase the stability of a protein by decreasing the entropic difference between the unfolded and the folded form.

The stem region of influenza B HA polypeptides consists of a fusion peptide, a N-terminal refolding region 1 (RR1), a central helix, and a C-terminal refolding region 2 (RR2). In some embodiments, the at least one proline mutation is introduced in the fusion peptide of the stem region. In some embodiments, the at least one proline mutation is introduced in the N-terminal refolding region 1 of the stem region. In some embodiments, the at least one proline mutation is introduced in the central helix of the stem region. In some embodiments, the at least one proline mutation is introduced in the C-terminal refolding region 2 of the stem region.

In some embodiments, the modified influenza B HA polypeptides of the present disclosure comprise one or more proline mutations at amino acid positions 363, 366, 371, 372, 376, 380, 383, 390, 391, 393, 395, 397, 399, 421, 430, 431, 434, 436, and/or 490 as indexed by reference to the amino acid sequence of SEQ ID NO: 1. In some embodiments, the modified influenza B HA polypeptides of the present disclosure comprise one or more proline mutations at amino acid positions 372, 397, 399, 421, 430, 431, 434, and/or 436 as indexed by reference to the amino acid sequence of SEQ ID NO: 1. Specifically, relative to a corresponding wild-type influenza B HA polypeptide, the influenza B HA polypeptide may comprise one or more prefusion conformation-stabilizing proline substitutions at amino acid positions 363, 366, 371, 372, 376, 380, 383, 390, 391, 393, 395, 397, 399, 421, 430, 431, 434, 436, and/or 490 as indexed by reference to the amino acid sequence of SEQ ID NO: 1. For example, relative to a corresponding wild-type influenza B HA polypeptide, the influenza B HA polypeptide may comprise one or more prefusion conformation-stabilizing proline substitutions at amino acid positions 372, 397, 399, 421, 430, 431, 434, and/or 436 as indexed by reference to the amino acid sequence of SEQ ID NO: 1. In some embodiments, relative to a corresponding wild-type influenza B HA polypeptide, the influenza B HA polypeptide comprises two prefusion conformation-stabilizing proline substitutions at amino acid position 430 and 436 as indexed by reference to the amino acid sequence of SEQ ID NO: 1.

In some embodiments, relative to a corresponding wild-type influenza B HA polypeptide, the modified influenza B HA polypeptides of the present disclosure comprise one or more of the proline substitutions selected from F363P, A366P, L371P, E372P, E376P, A380P, H383P, A390P, H391P, V393P, V395P, A397P, L399P, V421P, A430P, M431P, L434P, N436P, and/or S490P, wherein the amino acid positions are as indexed by reference to the amino acid sequence of SEQ ID NO: 1. In some embodiments, relative to a corresponding wild-type influenza B HA polypeptide, the modified influenza B HA polypeptides of the present disclosure comprise one or more of the proline substitutions selected from E372P, A397P, L399P, V421P, A430P, M431P, L434P, and/or N436P, wherein the amino acid positions are as indexed by reference to the amino acid sequence of SEQ ID NO: 1.

For example, relative to a corresponding wild-type influenza B HA polypeptide, an influenza B HA polypeptide comprising proline substitutions at amino acid positions 430 and 436 (e.g., A430P and N436P), wherein the amino acid positions are as indexed by reference to the amino acid sequence of SEQ ID NO: 1, was found to be stabilized in the prefusion conformation. Accordingly, in particular embodiments, relative to a corresponding wild-type influenza B HA polypeptide, the influenza B HA polypeptide of the present disclosure comprises the proline substitutions A430P and N436P, wherein the amino acid positions are as indexed by reference to the amino acid sequence of SEQ ID NO: 1.

### Disulfide Bridge Forming Mutations

In some embodiments, the modified influenza B HA polypeptides provided herein comprise at least two cysteine mutations at appropriate amino acid positions such that the introduction of the at least two cysteine mutations form a disulfide bridge (or disulfide bond) in the modified influenza B HA polypeptide. Such mutations are referred to as “disulfide bridge forming mutations” throughout the present disclosure. Without wishing to be bound by any theory, the introduced disulfide bridge appears to stabilize the modified influenza B HA polypeptide's conformation state, such as prefusion conformation. For stabilizing the prefusion conformation of the modified influenza B HA polypeptide, the amino acid pair chosen for mutation to cysteine should be in close proximity in the prefusion conformation but distant in the postfusion conformation. Such amino acid pair can be identified, for example, by visual inspection of a crystal structure of an influenza B HA polypeptide in a prefusion conformation, such as that of influenza virus B/Yamanashi/166/1998 (PDB ID 4M40), or more quantitative selection using computational protein design software, such as BioLuminate™ (Schrodinger LLC, New York, 2015), Discovery Studio™ (Accelrys, San Diego, 2015), MOE™ (Chemical Computing Group Inc., Montreal, 2015), and Rosetta™ (University of Washington, Seattle, 2015).

In some embodiments, the modified influenza B HA polypeptides provided herein comprise at least two cysteine mutations that are appropriately positioned so that the at least two cysteine mutations form a disulfide bridge linking HA1 and HA2 of the modified influenza B HA polypeptide to stabilize the prefusion conformation and reduce or eliminate postfusion conformation. In some embodiments, the modified influenza B HA polypeptides provided herein comprise at least two cysteine mutations that are appropriately positioned so that the at least two cysteine mutations form a disulfide bridge between a loop region of HA1 and a helix of HA2 in the modified influenza B HA polypeptide. In some embodiments, the modified influenza B HA polypeptides provided herein comprise at least two cysteine mutations that are appropriately positioned so that the at least two cysteine mutations form a disulfide bridge in the stem region of the modified influenza B HA polypeptide.

In some embodiments, the modified influenza B HA polypeptides of the disclosure comprise at least two cysteine mutations at amino acid positions 20 and 387, 35 and 408, 36 and 415, 37 and 411, 125 and 431, 127 and 431, 185 and 223, 186 and 224, 186 and 239, 188 and 241, 232 and 433, 233 and 434, 239 and 276, 346 and 465, 367 and 478, 378 and 397, 380 and 397, 383 and 401, 387 and 510, 394 and 507, 394 and 510, 396 and 510, 396 and 514, 401 and 475, 430 and 437, 430 and 438, and/or 430 and 439, as indexed by reference to the amino acid sequence of SEQ ID NO: 1. In some embodiments, the modified influenza B HA polypeptides of the disclosure comprise at least two cysteine mutations at amino acid positions 20 and 387, 36 and 415, 125 and 431, 127 and 431, 185 and 223, 186 and 224, 186 and 239, 188 and 241, 233 and 434, 239 and 276, 346 and 465, 367 and 478, 378 and 397, 383 and 401, 387 and 510, 394 and 507, 394 and 510, 396 and 510, 401 and 475, 430 and 437, 430 and 438, and/or 430 and 439, as indexed by reference to the amino acid sequence of SEQ ID NO: 1. In some embodiments, the modified influenza B HA polypeptides of the disclosure comprise at least two cysteine mutations at amino acid positions 383 and 401, and/or amino acid positions 401 and 475, as indexed by reference to the amino acid sequence of SEQ ID NO: 1.

Specifically, relative to a corresponding wild-type influenza B HA polypeptide, the influenza B HA polypeptide may comprise at least two disulfide bridge-forming cysteine substitutions at amino acid positions 20 and 387, 35 and 408, 36 and 415, 37 and 411, 125 and 431, 127 and 431, 185 and 223, 186 and 224, 186 and 239, 188 and 241, 232 and 433, 233 and 434, 239 and 276, 346 and 465, 367 and 478, 378 and 397, 380 and 397, 383 and 401, 387 and 510, 394 and 507, 394 and 510, 396 and 510, 396 and 514, 401 and 475, 430 and 437, 430 and 438, and/or 430 and 439, as indexed by reference to the amino acid sequence of SEQ ID NO: 1. For example, relative to a corresponding wild-type influenza B HA polypeptide, the influenza B HA polypeptide may comprise at least two disulfide bridge-forming cysteine substitutions at amino acid positions 20 and 387, 36 and 415, 125 and 431, 127 and 431, 185 and 223, 186 and 224, 186 and 239, 188 and 241, 233 and 434, 239 and 276, 346 and 465, 367 and 478, 378 and 397, 383 and 401, 387 and 510, 394 and 507, 394 and 510, 396 and 510, 401 and 475, 430 and 437, 430 and 438, and/or 430 and 439, as indexed by reference to the amino acid sequence of SEQ ID NO: 1. In particular embodiments, relative to a corresponding wild-type influenza B HA polypeptide, the influenza B HA polypeptide comprises at least two disulfide bridge-forming cysteine substitutions at amino acid positions 383 and 401, and/or amino acid positions 401 and 475, as indexed by reference to the amino acid sequence of SEQ ID NO: 1.

In some embodiments, relative to a corresponding wild-type influenza B HA polypeptide, the modified influenza B HA polypeptides of the disclosure comprise at least two cysteine substitutions selected from I20C and S387C, T35C and K408C, A36C and S415C, G37C and K411C, L125C and M431C, L127C and M431C, N185C and K223C, P186C and P224C, P186C and V239C, T188C and Q241C, N232C and E433C, G233C and L434C, V239C and T276C, A346C and N465C, I367C and A478C, M378C and A397C, A380C and A397C, H383C and S401C, S387C and L510C, A394C and Q507C, A394C and L510C, A396C and L510C, A396C and A514C, S401C and H475C, A430C and E437C, A430C and I438C, and/or A430C and L439C, wherein the amino acid positions are as indexed by reference to the amino acid sequence of SEQ ID NO: 1. In some embodiments, relative to a corresponding wild-type influenza B HA polypeptide, the modified influenza B HA polypeptides of the disclosure comprise at least two cysteine substitutions selected from I20C and S387C, A36C and S415C, L125C and M431C, L127C and M431C, N185C and K223C, P186C and P224C, P186C and V239C, T188C and Q241C, G233C and L434C, V239C and T276C, A346C and N465C, I367C and A478C, M378C and A397C, H383C and S401C, S387C and L510C, A394C and Q507C, A394C and L510C, A396C and L510C, S401C and H475C, A430C and E437C, A430C and I438C, and/or A430C and L439C, wherein the amino acid positions are as indexed by reference to the amino acid sequence of SEQ ID NO: 1.

For example, relative to a corresponding wild-type influenza B HA polypeptide, the influenza B HA polypeptides comprising disulfide bridge-forming cysteine substitutions at amino acid positions 383 and 401 (e.g., H383C and S401C) and 401 and 475 (e.g., S401C and H475C), wherein the amino acid positions are indexed by reference to the amino acid sequence of SEQ ID NO: 1, were found to be stabilized in the prefusion conformation. Accordingly, in particular embodiments, relative to a corresponding wild-type influenza B HA polypeptide, the influenza B HA polypeptide comprises disulfide bridge-forming cysteine substitutions at amino acid positions 383 and 401, or amino acid positions 401 and 475, wherein the amino acid positions are as indexed by reference to the amino acid sequence of SEQ ID NO: 1.

### Interface Stabilizing Mutations

In some embodiments, relative to a corresponding wild-type influenza B HA polypeptide, the modified influenza B HA polypeptides provided herein comprise one or more amino acid mutations (e.g., one or more amino acid substitutions) in the head (comprising amino acid residues 57-307 as indexed by reference to the amino acid sequence of SEQ ID NO: 1) and/or stem region (comprising amino acid residues 16-56 and 308-547 as indexed by reference to the amino acid sequence of SEQ ID NO: 1), wherein the one or more amino acid mutations stabilize the modified influenza B HA polypeptides in a prefusion conformation through interface stabilization. Such mutations are also referred to as “interface stabilizing mutations” throughout the present disclosure. Without wishing to be bound by any theory, interface stabilization can be achieved through various ways, such as cavity filling and formation of polar interaction with a neighboring residue (e.g., formation of a hydrogen bond or salt bridge). Accordingly, in some embodiments, the one or more amino acid mutations that stabilize the modified influenza B HA polypeptides in a prefusion conformation through interface stabilization comprise at least one cavity filling mutation in the stem region. In some embodiments, the one or more amino acid mutations that stabilize the modified influenza B HA polypeptides in a prefusion conformation through interface stabilization comprise one or more amino acid mutations in the head and/or stem region to form a polar interaction with a neighboring amino acid residue. In some embodiments, the polar interaction comprises a hydrogen bond. In some embodiments, the polar interaction comprises a salt bridge.

The term “cavity filling mutation” refers to a mutation that results in a substitution of an amino acid residue in a parental polypeptide, such as a wild-type influenza B HA polypeptide (e.g., HA polypeptide of B/PHUKET/3073/2013 or B/Austria/1359417/2021) by an amino acid that is expected to fill an internal cavity (i.e., a void space) that exists in the folded structure of the parental polypeptide. Without wishing to be bound by any theory, such cavity filling mutations can contribute to stabilizing the pH-sensitive interfaces between the head and stem regions and thus, stabilize the prefusion conformation of the modified influenza B HA polypeptide. The cavities in the prefusion conformation of a wild-type influenza B HA polypeptide can be identified, for example, by visual inspection of a crystal structure of an influenza B HA polypeptide in a prefusion conformation, such as that of influenza virus B/Yamanashi/166/1998 (PDB ID 4M40), or more quantitative selection using computational protein design software, such as BioLuminate™ (Schrodinger LLC, New York, 2015), Discovery Studio™ (Accelrys, San Diego, 2015), MOE™ (Chemical Computing Group Inc., Montreal, 2015), and Rosetta™ (University of Washington, Seattle, 2015). The amino acids to be substituted for cavity filling mutations typically include small aliphatic amino acids (e.g., Glycine (G), Alanine (A), and Valine (V)) or small polar amino acids (e.g., Serine (S) and Threonine (T)). They may also include amino acids that are buried in the prefusion conformation but exposed to solvent in the postfusion conformation. The amino acid substitutions to be introduced can be large aliphatic amino acids (e.g., Isoleucine (I), Leucine (L) and Methionine (M)) or large aromatic amino acids (e.g., Histidine (H), Phenylalanine (F), Tyrosine (Y) and Tryptophan (W)) or an amino acid with a basic side chain at neutral pH, (e.g., Arginine (R), Lysine (K), and Histidine (H)). For example, Lysine (K) mutations can be introduced in the stem region to fill the cavity among central helices and to form polar interaction with a neighboring residue.

In some embodiments, relative to a corresponding wild-type influenza B HA polypeptide, the modified influenza B HA polypeptides of the disclosure comprise at least one interface stabilizing (e.g., cavity filling) mutation (e.g., a substitution) at amino acid position 460 as indexed by reference to the amino acid sequence of SEQ ID NO: 1. In some embodiments, the modified influenza B HA polypeptides of the disclosure comprise at least one interface stabilizing (e.g., cavity filling) mutation by substituting the amino acid at position 460 as indexed by reference to the amino acid sequence of SEQ ID NO: 1 with Histidine (H), Phenylalanine (F), or Lysine (K). In some embodiments, the modified influenza B HA polypeptides of the disclosure comprise at least one interface stabilizing (e.g., cavity filling) mutation comprising a substitution of the amino acid at position 460 as indexed by reference to the amino acid sequence of SEQ ID NO: 1 with Lysine (K).

In some embodiments, the amino acid at position 460 of the modified influenza B HA polypeptides, as indexed by reference to the amino acid sequence of SEQ ID NO: 1, is not substituted with Methionine (M), Leucine (L), Tryptophan (W), Tyrosine (Y), or Arginine (R). In some embodiments, the amino acid at position 460 of the modified influenza B HA polypeptides, as indexed by reference to the amino acid sequence of SEQ ID NO: 1, is not substituted with Arginine (R).

In some embodiments, relative to a corresponding wild-type influenza B HA polypeptide, the modified influenza B HA polypeptides of the disclosure comprise at least one interface stabilizing (e.g., cavity filling) mutation (e.g., a substitution) at amino acid position 467 as indexed by reference to the amino acid sequence of SEQ ID NO: 1. In some embodiments, the modified influenza B HA polypeptides of the disclosure comprise at least one interface stabilizing (e.g., cavity filling) mutation by substituting the amino acid at position 467 as indexed by reference to the amino acid sequence of SEQ ID NO: 1 with Histidine (H), Phenylalanine (F), Glutamine (Q), or Tyrosine (Y). In some embodiments, the modified influenza B HA polypeptides of the disclosure comprise at least one interface stabilizing (e.g., cavity filling) mutation by substituting the amino acid at position 467 as indexed by reference to the amino acid sequence of SEQ ID NO: 1 with Phenylalanine (F) or Tyrosine (Y).

In some embodiments, relative to a corresponding wild-type influenza B HA polypeptide, the modified influenza B HA polypeptides of the disclosure comprise at least one interface stabilizing (e.g., cavity filling) mutation (e.g., a substitution) at amino acid position 474 as indexed by reference to the amino acid sequence of SEQ ID NO: 1. In some embodiments, the modified influenza B HA polypeptides of the disclosure comprise at least one interface stabilizing (e.g., cavity filling) mutation by substituting the amino acid at position 474 as indexed by reference to the amino acid sequence of SEQ ID NO: 1 with Phenylalanine (F), Asparagine (N), Glutamine (Q), or Tyrosine (Y). In some embodiments, the modified influenza B HA polypeptides of the disclosure comprise at least one interface stabilizing (e.g., cavity filling) mutation by substituting the amino acid at position 474 as indexed by reference to the amino acid sequence of SEQ ID NO: 1 with Glutamine (Q).

In some embodiments, relative to a corresponding wild-type influenza B HA polypeptide, the modified influenza B HA polypeptides of the disclosure comprise at least one interface stabilizing (e.g., cavity filling) substitution selected from A460K, G467F, G467Q, G467Y, and E474Q, wherein the amino acid position is as indexed by reference to the amino acid sequence of SEQ ID NO: 1.

In some embodiments, relative to a corresponding wild-type influenza B HA polypeptide, the one or more amino acid mutations (e.g., substitutions) that stabilize the modified influenza B HA polypeptide in the prefusion conformation through interface stabilization comprise one or more substitutions at amino acid positions 18, 121, 188, 226, 228, 408, 435, and/or 460 as indexed by reference to the amino acid sequence of SEQ ID NO: 1. In some embodiments, the one or more amino acid mutations that stabilize the modified influenza B HA polypeptide in the prefusion conformation through interface stabilization comprise one or more substitutions selected from D18W, Q121K, T188N, K226M, T228V, K408R, H435L, and A460K, wherein the amino acid position is as indexed by reference to the amino acid sequence of SEQ ID NO: 1.

### pH Sensor Inactivating Mutations

In some embodiments, relative to a corresponding wild-type influenza B HA polypeptide, the modified influenza B HA polypeptides provided herein comprise one or more amino acid mutations (e.g., one or more amino acid substitutions) in the head (comprising amino acid residues 57-307 as indexed by reference to the amino acid sequence of SEQ ID NO: 1) and/or stem region (comprising amino acid residues 16-56 and 308-547 as indexed by reference to the amino acid sequence of SEQ ID NO: 1), wherein the introduction of the one or more amino acid mutations inactivate one or more pH sensors in the head and/or stem region. Such mutations are also referred to as “pH sensor inactivating mutations” or “pH sensor knockout mutations” throughout the present disclosure. The transition of an influenza B HA polypeptide from the high-energy, metastable prefusion state to a postfusion conformation is triggered by low pH. Thus, without wishing to be bound by any theory, inactivating one or more pH sensors of the influenza B HA polypeptides may contribute to stabilizing the prefusion conformation of the modified influenza B HA polypeptide. Any amino acid residue or combination of residues acting as a pH sensor in the influenza B HA polypeptide known in the art or identified in the future can be substituted to inactivate such a pH sensor. For example, pH sensors in the stem region and/or at the head interface can be inactivated by substituting Histidine (H) with a hydrophobic residue such as Leucine (L) or Methionine (M).

In some embodiments, relative to a corresponding wild-type influenza B HA polypeptide, the one or more amino acid mutations (e.g., the one or more amino acid substitutions) that inactivate one or more pH sensors in the head and/or stem region of the modified influenza B HA polypeptides of the disclosure are at amino acid positions 226, 228, 237, 383, 388, 391, 401, 405, 408, 435, 460, 474 and/or 475 as indexed by reference to the amino acid sequence of SEQ ID NO: 1. In some embodiments, the one or more amino acid mutations (e.g., the one or more amino acid substitutions) that inactivate one or more pH sensors in the head and/or stem region of the modified influenza B HA polypeptides of the disclosure are at amino acid positions 226, 228, 237, 383, 388, 401, 405, 408, 435, 460, and/or 475 as indexed by reference to the amino acid sequence of SEQ ID NO: 1. In some embodiments, the one or more amino acid mutations (e.g., the one or more amino acid substitutions) that inactivate one or more pH sensors in the head and/or stem region of the modified influenza B HA polypeptides of the disclosure are at amino acid positions 226, 228, 237, 383, 388, 401, 408, 435, 460, and/or 475 as indexed by reference to the amino acid sequence of SEQ ID NO: 1.

In some embodiments, relative to a corresponding wild-type influenza B HA polypeptide, the modified influenza B HA polypeptides of the disclosure comprise one or more amino acid mutations (e.g., one or more amino acid substitutions) in the head region (comprising amino acid residues 57-307 as indexed by reference to the amino acid sequence of SEQ ID NO: 1), wherein the introduction of the one or more amino acid mutations inactivate one or more pH sensors in the head region. In such embodiments, the mutations can be substitutions at amino acid positions 226, 228, and/or 237 as indexed by reference to the amino acid sequence of SEQ ID NO: 1. Accordingly, in some embodiments, the modified influenza B HA polypeptides of the disclosure comprise a pH sensor inactivating mutation in the head region, the mutation comprising a substitution at amino acid position 226 as indexed by reference to the amino acid sequence of SEQ ID NO: 1. In certain embodiments, the amino acid at position 226 is substituted with Glutamine (Q) or Methionine (M). In some embodiments, the amino acid at position 226 is substituted with Methionine (M). In some embodiments, the modified influenza B HA polypeptides of the disclosure comprise a pH sensor inactivating mutation, the mutation comprising a substitution at amino acid position 228 as indexed by reference to the amino acid sequence of SEQ ID NO: 1. In some embodiments, the amino acid at position 228 is substituted with Valine (V). In some embodiments, the modified influenza B HA polypeptides of the disclosure comprise a pH sensor inactivating mutation, the mutation comprising a substitution at amino acid position 237 as indexed by reference to the amino acid sequence of SEQ ID NO: 1. In certain embodiments, the amino acid at position 237 is substituted with Isoleucine (I) or Leucine (L). In some embodiments, the amino acid at position 237 is substituted with Leucine (L).

In some embodiments, relative to a corresponding wild-type influenza B HA polypeptide, the modified influenza B HA polypeptides of the disclosure comprise at least one pH sensor inactivating mutation (e.g., at least one amino acid substitution) in the head region (comprising amino acid residues 57-307 as indexed by reference to the amino acid sequence of SEQ ID NO: 1), the at least one pH sensor inactivating mutation comprising at least two substitutions at amino acid positions 226 and 237 as indexed by reference to the amino acid sequence of SEQ ID NO: 1. In certain embodiments, the amino acids at positions 226 and 237 are substituted with Glutamine (Q) and Isoleucine (I), respectively. In some embodiments, the amino acids at positions 226 and 237 are substituted with Glutamine (Q) and Leucine (L), respectively. In some embodiments, the amino acids at positions 226 and 237 are substituted with Methionine (M) and Isoleucine (I), respectively. In some embodiments, the amino acids at positions 226 and 237 are substituted with Methionine (M) and Leucine (L), respectively.

In some embodiments, relative to a corresponding wild-type influenza B HA polypeptide, the modified influenza B HA polypeptides of the disclosure comprise at least one pH sensor inactivating mutation (e.g., at least one amino acid substitution) in the head region (comprising amino acid residues 57-307 as indexed by reference to the amino acid sequence of SEQ ID NO: 1), the at least one pH sensor inactivating mutation comprising at least two substitutions at amino acid positions 226 and 228 as indexed by reference to the amino acid sequence of SEQ ID NO: 1. In some embodiments, the amino acids at positions 226 and 228 are substituted with Methionine (M) and Valine (V), respectively.

In some embodiments, relative to a corresponding wild-type influenza B HA polypeptide, the modified influenza B HA polypeptides of the disclosure comprise one or more amino acid mutations (e.g., one or more amino acid substitutions) in the stem region (comprising amino acid residues 16-56 and 308-547 as indexed by reference to the amino acid sequence of SEQ ID NO: 1), wherein the introduction of the one or more amino acid mutations inactivate one or more pH sensors in the stem region. In such embodiments, the mutations can be substitutions at amino acid positions 383, 388, 391, 401, 405, 408, 435, 460, 474, and/or 475 as indexed by reference to the amino acid sequence of SEQ ID NO: 1. Accordingly, in some embodiments, the modified influenza B HA polypeptides of the disclosure comprise a pH sensor inactivating mutation in the stem region, the mutation comprising a substitution at amino acid position 383 as indexed by reference to the amino acid sequence of SEQ ID NO: 1. In certain embodiments, the amino acid at position 383 is substituted with Isoleucine (I), Leucine (L), Methionine (M), or Proline (P). In some embodiments, the amino acid at position 383 is substituted with Leucine (L) or Methionine (M). In some embodiments, the modified influenza B HA polypeptides of the disclosure comprise a pH sensor inactivating mutation in the stem region, the mutation comprising a substitution at amino acid position 388 as indexed by reference to the amino acid sequence of SEQ ID NO: 1. In some embodiments, the amino acid at position 388 is substituted with Tryptophan (W). In some embodiments, the modified influenza B HA polypeptides of the disclosure comprise a pH sensor inactivating mutation in the stem region, the mutation comprising a substitution at amino acid position 391 as indexed by reference to the amino acid sequence of SEQ ID NO: 1. In certain embodiments, the amino acid at position 391 is substituted with Proline (P). In some embodiments, the modified influenza B HA polypeptides of the disclosure comprise a pH sensor inactivating mutation in the stem region, the mutation comprising a substitution at amino acid position 401 as indexed by reference to the amino acid sequence of SEQ ID NO: 1. In certain embodiments, the amino acid at position 401 is substituted with Isoleucine (I) or Valine (V). In some embodiments, the amino acid at position 401 is substituted with Valine (V). In some embodiments, the modified influenza B HA polypeptides of the disclosure comprise a pH sensor inactivating mutation in the stem region, the mutation comprising a substitution at amino acid position 405 as indexed by reference to the amino acid sequence of SEQ ID NO: 1. In certain embodiments, the amino acid at position 405 is substituted with Ile or Valine (V). In some embodiments, the modified influenza B HA polypeptides of the disclosure comprise a pH sensor inactivating mutation in the stem region, the mutation comprising a substitution at amino acid position 408 as indexed by reference to the amino acid sequence of SEQ ID NO: 1. In certain embodiments, the amino acid at position 408 is substituted with Methionine (M). In some embodiments, the modified influenza B HA polypeptides of the disclosure comprise a pH sensor inactivating mutation in the stem region, the mutation comprising a substitution at amino acid position 435 as indexed by reference to the amino acid sequence of SEQ ID NO: 1. In certain embodiments, the amino acid at position 435 is substituted with Glutamic acid (E), Tyrosine (Y), or Phenylalanine (F). In some embodiments, the modified influenza B HA polypeptides of the disclosure comprise a pH sensor inactivating mutation in the stem region, the mutation comprising a substitution at amino acid position 460 as indexed by reference to the amino acid sequence of SEQ ID NO: 1. In certain embodiments, the amino acid at position 460 is substituted with Lysine (K). In some embodiments, the modified influenza B HA polypeptides of the disclosure comprise a pH sensor inactivating mutation in the stem region, the mutation comprising a substitution at amino acid position 474 as indexed by reference to the amino acid sequence of SEQ ID NO: 1. In certain embodiments, the amino acid at position 474 is substituted with Glutamine (Q). In some embodiments, the modified influenza B HA polypeptides of the disclosure comprise a pH sensor inactivating mutation in the stem region, the mutation comprising a substitution at amino acid position 475 as indexed by reference to the amino acid sequence of SEQ ID NO: 1. In certain embodiments, the amino acid at position 475 is substituted with Isoleucine (I), Leucine (L), Methionine (M), Tyrosine (Y), or Tryptophan (W). In some embodiments, the amino acid at position 475 is substituted with Tyrosine (Y) or Tryptophan (W).

In some embodiments, relative to a corresponding wild-type influenza B HA polypeptide, the modified influenza B HA polypeptides of the disclosure comprise at least one pH sensor inactivating mutation in the stem region, the at least one pH sensor inactivating mutation comprising at least two substitutions at amino acid positions 383 and 475 as indexed by reference to the amino acid sequence of SEQ ID NO: 1. In certain embodiments, both amino acids at positions 383 and 475 are substituted with Isoleucine (I). In some embodiments, both amino acids at positions 383 and 475 are substituted with Leucine (L). In some embodiments, both amino acids at positions 383 and 475 are substituted with Methionine (M). In some embodiments, the amino acids at positions 383 and 475 are substituted with Isoleucine (I) and Leucine (L), respectively. In some embodiments, the amino acids at positions 383 and 475 are substituted with Isoleucine (I) and Methionine (M), respectively. In some embodiments, the amino acids at positions 383 and 475 are substituted with Leucine (L) and Isoleucine (I), respectively. In some embodiments, the amino acids at positions 383 and 475 are substituted with Leucine (L) and Methionine (M), respectively. In some embodiments, the amino acids at positions 383 and 475 are substituted with Methionine (M) and Isoleucine (I), respectively. In some embodiments, the amino acids at positions 383 and 475 are substituted with Methionine (M) and Leucine (L), respectively.

In some embodiments, the modified influenza B HA polypeptides of the disclosure comprise at least one pH sensor inactivating mutation in the stem region, the at least one pH sensor inactivating mutation comprising at least two substitutions at amino acid positions 383 and 401 as indexed by reference to the amino acid sequence of SEQ ID NO: 1. In some embodiments, the amino acids at positions 383 and 401 are substituted with Methionine (M) and Valine (V), respectively. In some embodiments, the amino acids at positions 383 and 401 are substituted with Leucine (L) and Valine (V), respectively.

In some embodiments, the modified influenza B HA polypeptides of the disclosure comprise at least one pH sensor inactivating mutation in the stem region, the at least one pH sensor inactivating mutation comprising at least two substitutions at amino acid positions 383 and 460 as indexed by reference to the amino acid sequence of SEQ ID NO: 1. In some embodiments, the amino acids at positions 383 and 460 are substituted with Leucine (L) and Lysine (K).

In some embodiments, relative to a corresponding wild-type influenza B HA polypeptide, the modified influenza B HA polypeptides provided herein comprise one or more pH sensor inactivating substitutions selected from K226Q, K226M, T228V, H237I, H237L, H383I, H383L, H383M, H383P, H388W, H391P, S4011, S401V, A4051, A405V, K408M, H435E, H435F, A460K, E474Q, H475I, H475L, H475Y, H475W, and H475M, wherein the amino acid positions are as indexed by reference to the amino acid sequence of SEQ ID NO: 1. In some embodiments, relative to a corresponding wild-type influenza B HA polypeptide, the modified influenza B HA polypeptides provided herein comprise one or more pH sensor inactivating substitutions selected from K226M, T228V, H237L, H383M, H383L, H388W, S401V, K408M, H435E, H435F, H435Y, A460K, H475Y, and H475W, wherein the amino acid positions are as indexed by reference to the amino acid sequence of SEQ ID NO: 1. In some embodiments, the one or more pH sensor inactivating mutations in the modified influenza B HA polypeptides provided herein comprise amino acid substitutions K226M and H237L, wherein the amino acid positions are as indexed by reference to the amino acid sequence of SEQ ID NO: 1. In some embodiments, the one or more pH sensor inactivating mutations in the modified influenza B HA polypeptides provided herein comprise amino acid substitutions K226M and T228V, wherein the amino acid positions are as indexed by reference to the amino acid sequence of SEQ ID NO: 1. In some embodiments, the one or more pH sensor inactivating mutations in the modified influenza B HA polypeptides provided herein comprise amino acid substitutions H383M and S401V, wherein the amino acid positions are as indexed by reference to the amino acid sequence of SEQ ID NO: 1. In some embodiments, the one or more pH sensor inactivating mutations in the modified influenza B HA polypeptides provided herein comprise amino acid substitutions H383L and S401V, wherein the amino acid positions are as indexed by reference to the amino acid sequence of SEQ ID NO: 1. In some embodiments, the one or more pH sensor inactivating mutations in the modified influenza B HA polypeptides provided herein comprise amino acid substitutions H383L and A460K, wherein the amino acid positions are as indexed by reference to the amino acid sequence of SEQ ID NO: 1.

In some embodiments, relative to a corresponding wild-type influenza B HA polypeptide, the modified influenza B HA polypeptides provided herein comprise pH sensor inactivating substitutions at amino acid positions 383, 401, 405, 408, and 475, wherein the amino acid positions are as indexed by reference to the amino acid sequence of SEQ ID NO: 1. In some embodiments, relative to a corresponding wild-type influenza B HA polypeptide, the modified influenza B HA polypeptides provided herein comprise pH sensor inactivating substitutions H383M, S401V, A405V, K408M, and H475M, wherein the amino acid positions are as indexed by reference to the amino acid sequence of SEQ ID NO: 1.

### Glycan Engineering Mutations

The modified influenza B HA polypeptides of the disclosure can also comprise one or more mutations that results in one or more N-linked glycosylation motifs being introduced or disrupted. Such mutations are referred to as “glycan engineering mutations” throughout the present disclosure.

In some embodiments, relative to a corresponding wild-type influenza B HA polypeptide, the modified influenza B HA polypeptides of the disclosure comprise at least one amino acid mutation (e.g., at least one amino acid substitution) in the head region (comprising amino acid residues 57-307 as indexed by reference to the amino acid sequence of SEQ ID NO: 1), wherein the at least one amino acid mutation introduces at least one N-linked glycosylation motif in the head region. The at least one N-linked glycosylation motif introduced in the head region is located in or adjacent to the RBS of the head region in certain embodiments. The RBS of the head region can be defined as being a region of the modified influenza B HA polypeptide made up of residues at amino acid positions 110, 151-157, 165, 173-175, 208-218, 248, and 254-259 as indexed by reference to the amino acid sequence of SEQ ID NO: 1. Alternatively, the RBS may be defined as an area including all amino acid residues in the modified influenza B HA polypeptide that correspond to all amino acid residues within 5 angstroms of the LSTa molecule in the crystal structure of HA of influenza virus B/Yamanashi/166/1998 in complex with LSTa (PDB ID 4M40). Without wishing to be bound by any theory, adding additional N-linked glycosylation motif(s) to the head region, particularly in or adjacent to the RBS, can add additional glycan(s) to that region to mask non-neutralizing epitopes presented in the region, and promote a neutralizing response by the modified influenza B HA polypeptides. In some embodiments, the N-linked glycosylation motif comprises a consensus sequence of NxS/Ty, in which N is Asparagine, S/T is a Serine or Threonine residue, and x and y are any residue except Proline (P).

In some embodiments, relative to a corresponding wild-type influenza B HA polypeptide, the at least one N-linked glycosylation motif introduced in the head region (comprising amino acid residues 57-307 as indexed by reference to the amino acid sequence of SEQ ID NO: 1) of the modified influenza B HA polypeptides of the disclosure is generated by introducing one or more substitutions at amino acid positions 60, 62, 141, 143, 186, 187, 214, 216, 223, and/or 224 as indexed by reference to the amino acid sequence of SEQ ID NO: 1. In some embodiments, the amino acid at position 60 is substituted with Threonine (T). In some embodiments, the amino acid at position 62 is substituted with Threonine (T). In some embodiments, the amino acid at position 141 is substituted with Asparagine (N). In some embodiments, the amino acid at position 143 is substituted with Threonine (T). In some embodiments, the amino acid at position 186 is substituted with Phenylalanine (F). In some embodiments, the amino acid at position 187 is substituted with Threonine (T). In some embodiments, the amino acid at position 214 is substituted with Asparagine (N). In some embodiments, the amino acid at position 216 is substituted with Threonine (T). In some embodiments, the amino acid at position 223 is substituted with Asparagine (N). In some embodiments, the amino acid at position 224 is substituted with Phenylalanine (F).

In some embodiments, the at least one N-linked glycosylation motif introduced in the head region of the modified influenza B HA polypeptides of the disclosure is generated by amino acid substitutions selected from K60T, K62T, D141N, E143T, P186F, L187T, Q214N, K216T, K223N, and P224F. In some embodiments, the at least one N-linked glycosylation motif introduced in the head region of the modified influenza B HA polypeptides of the disclosure is generated by amino acid substitutions selected from K62T, P186F, L187T, Q214N, K216T, K223N, and P224F.

In some embodiments, relative to a corresponding wild-type influenza B HA polypeptide, the modified influenza B HA polypeptides of the disclosure further comprise at least one amino acid mutation (e.g., at least one amino acid substitution) in the stem region (comprising amino acid residues 16-56 and 308-547 as indexed by reference to the amino acid sequence of SEQ ID NO: 1), wherein the at least one amino acid mutation introduces or disrupts a N-linked glycosylation motif in the stem region. Without wishing to be bound by any theory, engineering N-linked glycosylation motif(s) in the stem region may help to stabilize the modified influenza B HA polypeptide in the prefusion conformation. In some embodiments, the N-linked glycosylation motif comprises a consensus sequence of NxS/Ty, in which N is Asparagine, S/T is a Serine or Threonine residue, and x and y are any residue except Proline (P).

In some embodiments, the at least one N-linked glycosylation motif introduced or disrupted in the stem region is generated by introducing at least one substitution at amino acid position 28, 336, and/or 349 as indexed by reference to the amino acid sequence of SEQ ID NO: 1. In some embodiments, the amino acid at position 28 is substituted with Threonine (T). In some embodiments, the amino acid at position 336 is substituted with Threonine (T). In certain embodiments, the amino acid at position 349 is substituted with Alanine (A) or Valine (V).

In some embodiments, the at least one N-linked glycosylation motif introduced or disrupted in the stem region of the modified influenza B HA polypeptides of the disclosure is generated by amino acid substitutions selected from P28T, K60T, P336T, T349A, and T349V, wherein the amino acid positions are as indexed by reference to the amino acid sequence of SEQ ID NO: 1.

In some embodiments, the modified influenza B HA polypeptides of the disclosure comprise at least one glycan engineering mutation in the head region and at least one glycan engineering mutation in the stem region. In some embodiments, the at least one glycan engineering mutation in the head region comprises at least one substitution at amino acid position 60 and the at least one glycan engineering mutation in the stem region comprises at least one substitution at amino acid position 28, as indexed by reference to the amino acid sequence of SEQ ID NO: 1. In some embodiments, the at least one glycan engineering mutation in the head region comprises amino acid substitution K60T and the at least one glycan engineering mutation in the stem region comprises amino acid substitution P28T.

### Sialic Acid Binding Interfering Mutations

In some embodiments, relative to a corresponding wild-type influenza B HA polypeptide, the modified influenza B HA polypeptides provided herein comprise at least one amino acid mutation (e.g., at least one amino acid substitution) in the head region (comprising amino acid residues 57-307 as indexed by reference to the amino acid sequence of SEQ ID NO: 1), wherein the at least one amino acid mutation reduces sialic acid binding of the modified influenza B HA polypeptide as compared to a control influenza B HA polypeptide without the at least one amino acid mutation that reduces sialic acid binding of the modified influenza B HA polypeptide. Such mutations are also referred to as “sialic acid binding interfering mutations” throughout the present disclosure. In some embodiments, the at least one amino acid mutation that reduces sialic acid binding of the modified influenza B HA polypeptide does not impair the integrity of the epitope targeted by RBS neutralizing antibodies. In certain embodiments, the at least one amino acid mutation (e.g., substitution) that reduces sialic acid binding of the modified influenza B HA polypeptide is at amino acid position 157, 177, 218, and/or 257 as indexed by reference to the amino acid sequence of SEQ ID NO: 1. In some embodiments, the amino acid at position 157 is substituted with Alanine (A). In some embodiments, the amino acid at position 177 is substituted with Aspartic Acid (D). In some embodiments, the amino acid at position 218 is substituted with Alanine (A). In some embodiments, the amino acid at position 257 is substituted with Leucine (L).

In some embodiments, the at least one sialic acid binding interfering mutation in the modified influenza B HA polypeptide of the disclosure comprises the amino acid substitution S257L, wherein the amino acid position is as indexed by reference to the amino acid sequence of SEQ ID NO: 1.

In some embodiments, the modified influenza B HA polypeptides provided herein comprise one or more sialic acid binding interfering substitutions selected from S157A, V177D, L218A, and S257L.

### Combinations of Mutations

In some embodiments, relative to a corresponding wild-type influenza B HA polypeptide, the modified influenza B HA polypeptides of the present disclosure comprise two or more (e.g., three, four, five, six, or seven) of the modifications (e.g., amino acid substitutions) selected from: a) at least one proline mutation (e.g., substitution) in the stem region (comprising amino acid residues 16-56 and 308-547 as indexed by reference to the amino acid sequence of SEQ ID NO: 1), wherein the at least one proline mutation breaks at least one helical structure in the stem region of the modified influenza B HA polypeptide in a postfusion conformation; b) at least two cysteine mutations (e.g., substitutions), wherein the at least two cysteine mutations form a disulfide bridge in the modified influenza B HA polypeptide; c) one or more amino acid mutations (e.g., substitutions) in the head (comprising amino acid residues 57-307 as indexed by reference to the amino acid sequence of SEQ ID NO: 1) and/or stem region (comprising amino acid residues 16-56 and 308-547 as indexed by reference to the amino acid sequence of SEQ ID NO: 1), wherein the one or more amino acid mutations stabilize the modified influenza B HA polypeptide in a prefusion conformation through interface stabilization; d) one or more amino acid mutations (e.g., substitutions) in the head (comprising amino acid residues 57-307 as indexed by reference to the amino acid sequence of SEQ ID NO: 1) and/or stem region (comprising amino acid residues 16-56 and 308-547 as indexed by reference to the amino acid sequence of SEQ ID NO: 1), wherein the one or more amino acid mutations inactivate one or more pH sensors in the head and/or stem region; e) at least one amino acid mutation (e.g., substitution) in the head region (comprising amino acid residues 57-307 as indexed by reference to the amino acid sequence of SEQ ID NO: 1), wherein the at least one amino acid mutation introduces at least one N-linked glycosylation motif in the head region; f) at least one amino acid mutation (e.g., substitution) in the stem region (comprising amino acid residues 16-56 and 308-547 as indexed by reference to the amino acid sequence of SEQ ID NO: 1), wherein the at least one amino acid mutation introduces or disrupts a N-linked glycosylation motif in the stem region; and g) at least one amino acid mutation (e.g., substitution) in the head region (comprising amino acid residues 57-307 as indexed by reference to the amino acid sequence of SEQ ID NO: 1), wherein the at least one amino acid mutation reduces sialic acid binding of the modified influenza B HA polypeptide as compared to a control influenza B HA polypeptide without the at least one amino acid mutation that reduces sialic acid binding of the modified influenza B HA polypeptide.

For instance, one or more pH sensor inactivating mutations (e.g., substitutions) may be combined with one or more interface stabilizing mutations (e.g., substitutions), or one or more glycan engineering amino acid mutations (e.g., substitutions) may be combined with one or more amino acid mutations (e.g., substitutions) that stabilize the modified influenza B HA polypeptide in the prefusion conformation or disrupt the postfusion conformation. Accordingly, in some embodiments, one or more modifications selected from options a)-d) above may be combined with one or more modifications of options e)-g) above. Alternatively or in addition, one or more modifications that disrupt the postfusion conformation may be combined with one or more modifications that stabilize the prefusion conformation of the modified influenza B HA polypeptide.

For example, in some embodiments, one or more proline mutations (e.g., substitutions) disclosed herein are combined with one or more disulfide bridge forming mutations (e.g., substitutions) disclosed herein. In some embodiments, one or more proline mutations (e.g., substitutions) disclosed herein are combined with one or more interface stabilizing mutations (e.g., substitutions) disclosed herein. In some embodiments, one or more proline mutations (e.g., substitutions) disclosed herein are combined with one or more pH sensor inactivating mutations (e.g., substitutions) disclosed herein. In some embodiments, one or more proline mutations (e.g., substitutions) disclosed herein are combined with one or more glycan engineering mutations (e.g., substitutions) in the head and/or stem regions as disclosed herein. In some embodiments, one or more proline mutations (e.g., substitutions) disclosed herein are combined with one or more sialic acid binding interfering mutations (e.g., substitutions) disclosed herein.

In some embodiments, one or more disulfide bridge forming mutations (e.g., substitutions) disclosed herein are combined with one or more interface stabilizing mutations (e.g., substitutions) disclosed herein. In some embodiments, one or more disulfide bridge forming mutations (e.g., substitutions) disclosed herein are combined with one or more pH sensor inactivating mutations (e.g., substitutions) disclosed herein. In some embodiments, one or more disulfide bridge forming mutations (e.g., substitutions) disclosed herein are combined with one or more glycan engineering mutations (e.g., substitutions) in the head and/or stem regions as disclosed herein. In some embodiments, one or more disulfide bridge forming mutations (e.g., substitutions) disclosed herein are combined with one or more sialic acid binding interfering mutations (e.g., substitutions) disclosed herein.

In some embodiments, one or more interface stabilizing mutations (e.g., substitutions) disclosed herein are combined with one or more pH sensor inactivating mutations (e.g., substitutions) disclosed herein. In some embodiments, one or more interface stabilizing mutations (e.g., substitutions) disclosed herein are combined with one or more glycan engineering mutations (e.g., substitutions) in the head and/or stem regions as disclosed herein. In some embodiments, one or more interface stabilizing mutations (e.g., substitutions) disclosed herein are combined with one or more sialic acid binding interfering mutations (e.g., substitutions) disclosed herein.

In some embodiments, one or more pH sensor inactivating mutations (e.g., substitutions) disclosed herein are combined with one or more glycan engineering mutations (e.g., substitutions) in the head and/or stem regions as disclosed herein. In some embodiments, one or more pH sensor inactivating mutations (e.g., substitutions) disclosed herein are combined with one or more sialic acid binding interfering mutations (e.g., substitutions) disclosed herein.

In some embodiments, one or more glycan engineering mutations (e.g., substitutions) in the head and/or stem regions as disclosed herein are combined with one or more sialic acid binding interfering mutations (e.g., substitutions) disclosed herein.

More than two different types of modifications can also be combined. For instance, in some embodiments, one or more proline mutations (e.g., substitutions) disclosed herein are combined with one or more disulfide bridge forming mutations (e.g., substitutions) disclosed herein and one or more interface stabilizing mutations (e.g., substitutions) disclosed herein. In some embodiments, one or more proline mutations (e.g., substitutions) disclosed herein are combined with one or more disulfide bridge forming mutations (e.g., substitutions) disclosed herein and one or more pH sensor inactivating mutations (e.g., substitutions) disclosed herein. In some embodiments, one or more proline mutations (e.g., substitutions) disclosed herein are combined with one or more disulfide bridge forming mutations (e.g., substitutions) disclosed herein and one or more glycan engineering mutations (e.g., substitutions) in the head and/or stem regions as disclosed herein. In some embodiments, one or more proline mutations (e.g., substitutions) disclosed herein are combined with one or more disulfide bridge forming mutations (e.g., substitutions) disclosed herein and one or more sialic acid binding interfering mutations (e.g., substitutions) disclosed herein. In some embodiments, one or more proline mutations (e.g., substitutions) disclosed herein are combined with one or more interface stabilizing mutations (e.g., substitutions) disclosed herein and one or more pH sensor inactivating mutations (e.g., substitutions) disclosed herein. In some embodiments, one or more proline mutations (e.g., substitutions) disclosed herein are combined with one or more interface stabilizing mutations (e.g., substitutions) disclosed herein and one or more glycan engineering mutations (e.g., substitutions) in the head and/or stem regions as disclosed herein. In some embodiments, one or more proline mutations (e.g., substitutions) disclosed herein are combined with one or more interface stabilizing mutations (e.g., substitutions) disclosed herein and one or more sialic acid binding interfering mutations (e.g., substitutions) disclosed herein. In some embodiments, one or more proline mutations (e.g., substitutions) disclosed herein are combined with one or more pH sensor inactivating mutations (e.g., substitutions) disclosed herein and one or more glycan engineering mutations (e.g., substitutions) in the head and/or stem regions as disclosed herein. In some embodiments, one or more proline mutations (e.g., substitutions) disclosed herein are combined with one or more pH sensor inactivating mutations (e.g., substitutions) disclosed herein and one or more sialic acid binding interfering mutations (e.g., substitutions) disclosed herein. In some embodiments, one or more proline mutations (e.g., substitutions) disclosed herein are combined with one or more glycan engineering mutations (e.g., substitutions) in the head and/or stem regions as disclosed herein and one or more sialic acid binding interfering mutations (e.g., substitutions) disclosed herein.

In some embodiments, one or more disulfide bridge forming mutations (e.g., substitutions) disclosed herein are combined with one or more interface stabilizing mutations (e.g., substitutions) disclosed herein and one or more pH sensor inactivating mutations (e.g., substitutions) disclosed herein. In some embodiments, one or more disulfide bridge forming mutations (e.g., substitutions) disclosed herein are combined with one or more interface stabilizing mutations (e.g., substitutions) disclosed herein and one or more glycan engineering mutations (e.g., substitutions) in the head and/or stem regions as disclosed herein. In some embodiments, one or more disulfide bridge forming mutations (e.g., substitutions) disclosed herein are combined with one or more interface stabilizing mutations (e.g., substitutions) disclosed herein and one or more sialic acid binding interfering mutations (e.g., substitutions) disclosed herein. In some embodiments, one or more disulfide bridge forming mutations (e.g., substitutions) disclosed herein are combined with one or more pH sensor inactivating mutations (e.g., substitutions) disclosed herein and one or more glycan engineering mutations (e.g., substitutions) in the head and/or stem regions as disclosed herein. In some embodiments, one or more disulfide bridge forming mutations (e.g., substitutions) disclosed herein are combined with one or more pH sensor inactivating mutations (e.g., substitutions) disclosed herein and one or more sialic acid binding interfering mutations (e.g., substitutions) disclosed herein. In some embodiments, one or more disulfide bridge forming mutations (e.g., substitutions) disclosed herein are combined with one or more glycan engineering mutations (e.g., substitutions) in the head and/or stem regions as disclosed herein and one or more sialic acid binding interfering mutations (e.g., substitutions) disclosed herein.

In some embodiments, one or more interface stabilizing mutations (e.g., substitutions) disclosed herein are combined with one or more pH sensor inactivating mutations (e.g., substitutions) disclosed herein and one or more glycan engineering mutations (e.g., substitutions) in the head and/or stem regions as disclosed herein. In some embodiments, one or more interface stabilizing mutations (e.g., substitutions) disclosed herein are combined with one or more pH sensor inactivating mutations (e.g., substitutions) disclosed herein and one or more sialic acid binding interfering mutations (e.g., substitutions) disclosed herein. In some embodiments, one or more interface stabilizing mutations (e.g., substitutions) disclosed herein are combined with one or more glycan engineering mutations (e.g., substitutions) in the head and/or stem regions as disclosed herein and one or more sialic acid binding interfering mutations (e.g., substitutions) disclosed herein.

In some embodiments, one or more pH sensor inactivating mutations (e.g., substitutions) disclosed herein are combined with one or more glycan engineering mutations (e.g., substitutions) in the head and/or stem regions as disclosed herein and one or more sialic acid binding interfering mutations (e.g., substitutions) disclosed herein.

In some embodiments, one or more proline mutations (e.g., substitutions) disclosed herein are combined with one or more disulfide bridge forming mutations (e.g., substitutions) disclosed herein, one or more interface stabilizing mutations (e.g., substitutions) disclosed herein, and one or more pH sensor inactivating mutations (e.g., substitutions) disclosed herein. In some embodiments, one or more proline mutations (e.g., substitutions) disclosed herein are combined with one or more disulfide bridge forming mutations (e.g., substitutions) disclosed herein, one or more interface stabilizing mutations (e.g., substitutions) disclosed herein, and one or more glycan engineering mutations (e.g., substitutions) in the head and/or stem regions as disclosed herein. In some embodiments, one or more proline mutations (e.g., substitutions) disclosed herein are combined with one or more disulfide bridge forming mutations (e.g., substitutions) disclosed herein, one or more interface stabilizing mutations (e.g., substitutions) disclosed herein, and one or more sialic acid binding interfering mutations (e.g., substitutions) disclosed herein. In some embodiments, one or more proline mutations (e.g., substitutions) disclosed herein are combined with one or more disulfide bridge forming mutations (e.g., substitutions) disclosed herein, one or more pH sensor inactivating mutations (e.g., substitutions) disclosed herein, and one or more glycan engineering mutations (e.g., substitutions) in the head and/or stem regions as disclosed herein. In some embodiments, one or more proline mutations (e.g., substitutions) disclosed herein are combined with one or more disulfide bridge forming mutations (e.g., substitutions) disclosed herein, one or more pH sensor inactivating mutations (e.g., substitutions) disclosed herein, and one or more sialic acid binding interfering mutations (e.g., substitutions) disclosed herein. In some embodiments, one or more proline mutations (e.g., substitutions) disclosed herein are combined with one or more disulfide bridge forming mutations (e.g., substitutions) disclosed herein, one or more glycan engineering mutations (e.g., substitutions) in the head and/or stem regions as disclosed herein, and one or more sialic acid binding interfering mutations (e.g., substitutions) disclosed herein. In some embodiments, one or more proline mutations (e.g., substitutions) disclosed herein are combined with one or more interface stabilizing mutations (e.g., substitutions) disclosed herein, one or more pH sensor inactivating mutations (e.g., substitutions) disclosed herein, and one or more glycan engineering mutations (e.g., substitutions) in the head and/or stem regions as disclosed herein. In some embodiments, one or more proline mutations (e.g., substitutions) disclosed herein are combined with one or more interface stabilizing mutations (e.g., substitutions) disclosed herein, one or more pH sensor inactivating mutations (e.g., substitutions) disclosed herein, and one or more sialic acid binding interfering mutations (e.g., substitutions) disclosed herein. In some embodiments, one or more proline mutations (e.g., substitutions) disclosed herein are combined with one or more pH sensor inactivating mutations (e.g., substitutions) disclosed herein, one or more glycan engineering mutations (e.g., substitutions) in the head and/or stem regions as disclosed herein, and one or more sialic acid binding interfering mutations (e.g., substitutions) disclosed herein.

In some embodiments, one or more disulfide bridge forming mutations (e.g., substitutions) disclosed herein are combined with one or more interface stabilizing mutations (e.g., substitutions) disclosed herein, one or more pH sensor inactivating mutations (e.g., substitutions) disclosed herein, and one or more glycan engineering mutations (e.g., substitutions) in the head and/or stem regions as disclosed herein. In some embodiments, one or more disulfide bridge forming mutations (e.g., substitutions) disclosed herein are combined with one or more interface stabilizing mutations (e.g., substitutions) disclosed herein, one or more pH sensor inactivating mutations (e.g., substitutions) disclosed herein, and one or more sialic acid binding interfering mutations disclosed herein. In some embodiments, one or more disulfide bridge forming mutations (e.g., substitutions) disclosed herein are combined with one or more pH sensor inactivating mutations (e.g., substitutions) disclosed herein, one or more glycan engineering mutations (e.g., substitutions) in the head and/or stem regions as disclosed herein, and one or more sialic acid binding interfering mutations (e.g., substitutions) disclosed herein.

In some embodiments, one or more interface stabilizing mutations (e.g., substitutions) disclosed herein are combined with one or more pH sensor inactivating mutations (e.g., substitutions) disclosed herein, one or more glycan engineering mutations (e.g., substitutions) in the head and/or stem regions as disclosed herein, and one or more sialic acid binding interfering mutations (e.g., substitutions) disclosed herein.

In some embodiments, one or more proline mutations (e.g., substitutions) disclosed herein are combined with one or more disulfide bridge forming mutations (e.g., substitutions) disclosed herein, one or more interface stabilizing mutations (e.g., substitutions) disclosed herein, one or more pH sensor inactivating mutations (e.g., substitutions) disclosed herein, and one or more glycan engineering mutations (e.g., substitutions) in the head and/or stem regions as disclosed herein. In some embodiments, one or more proline mutations (e.g., substitutions) disclosed herein are combined with one or more disulfide bridge forming mutations (e.g., substitutions) disclosed herein, one or more interface stabilizing mutations (e.g., substitutions) disclosed herein, one or more pH sensor inactivating mutations (e.g., substitutions) disclosed herein, and one or more sialic acid binding interfering mutations (e.g., substitutions) disclosed herein. In some embodiments, one or more proline mutations (e.g., substitutions) disclosed herein are combined with one or more interface stabilizing mutations (e.g., substitutions) disclosed herein, one or more pH sensor inactivating mutations (e.g., substitutions) disclosed herein, one or more glycan engineering mutations (e.g., substitutions) in the head and/or stem regions as disclosed herein, and one or more sialic acid binding interfering mutations (e.g., substitutions) disclosed herein.

In some embodiments, one or more disulfide bridge forming mutations (e.g., substitutions) disclosed herein are combined with one or more interface stabilizing mutations (e.g., substitutions) disclosed herein, one or more pH sensor inactivating mutations (e.g., substitutions) disclosed herein, one or more glycan engineering mutations (e.g., substitutions) in the head and/or stem regions as disclosed herein, and one or more sialic acid binding interfering mutations (e.g., substitutions) disclosed herein. In some embodiments, one or more proline mutations (e.g., substitutions) disclosed herein are combined with one or more disulfide bridge forming mutations (e.g., substitutions) disclosed herein, one or more interface stabilizing mutations (e.g., substitutions) disclosed herein, one or more pH sensor inactivating mutations (e.g., substitutions) disclosed herein, one or more glycan engineering mutations (e.g., substitutions) in the head and/or stem regions as disclosed herein, and one or more sialic acid binding interfering mutations (e.g., substitutions) disclosed herein.

In particular embodiments, the influenza B HA polypeptides provided herein comprise: a) at least one proline substitution relative to a corresponding wild-type influenza B HA polypeptide, wherein the at least one proline substitution is at amino acid position 363, 366, 371, 372, 376, 380, 383, 390, 391, 393, 395, 397, 399, 421, 430, 431, 434, 436, and/or 490 as indexed by reference to the amino acid sequence of SEQ ID NO: 1; b) at least two cysteine substitutions relative to a corresponding wild-type influenza B HA polypeptide, wherein the at least two cysteine substitutions are at amino acid positions 20 and 387, 35 and 408, 36 and 415, 37 and 411, 125 and 431, 127 and 431, 185 and 223, 186 and 224, 186 and 239, 188 and 241, 232 and 433, 233 and 434, 239 and 276, 346 and 465, 367 and 478, 378 and 397, 380 and 397, 383 and 401, 387 and 510, 394 and 507, 394 and 510, 396 and 510, 396 and 514, 401 and 475, 430 and 437, 430 and 438, and/or 430 and 439, as indexed by reference to the amino acid sequence of SEQ ID NO: 1; c) at least one cavity filling amino acid substitution relative to a corresponding wild-type influenza B HA polypeptide, wherein the at least one cavity filling amino acid substitution is at amino acid position 460, 467, and/or 474 as indexed by reference to the amino acid sequence of SEQ ID NO: 1; d) one or more interface stabilization amino acid substitutions relative to a corresponding wild-type influenza B HA polypeptide, wherein the one or more interface stabilization amino acid substitutions are at amino acid position 18, 121, 188, 226, 228, 408, 435, and/or 460 as indexed by reference to the amino acid sequence of SEQ ID NO: 1; e) one or more pH sensor knock-out amino acid substitutions relative to a corresponding wild-type influenza B HA polypeptide, wherein the one or more pH sensor knock-out amino acid substitutions are at amino acid position 226, 228, 237, 239, 383, 388, 391, 401, 405, 408, 435, 460, 474 and/or 475 as indexed by reference to the amino acid sequence of SEQ ID NO: 1; f) at least one amino acid substitution relative to a corresponding wild-type influenza B HA polypeptide, wherein the at least one amino acid substitution generates or disrupts a N-linked glycosylation motif in the influenza B HA polypeptide and is at amino acid position 28, 60, 62, 141, 143, 186, 187, 214, 216, 223, 224, 336, and/or 349 as indexed by reference to the amino acid sequence of SEQ ID NO: 1; and/or g) at least one amino acid substitution relative to a corresponding wild-type influenza B HA polypeptide, wherein the at least one amino acid substitution is at amino acid position 157, 177, 218, and/or 257 as indexed by reference to the amino acid sequence of SEQ ID NO: 1.

### Exemplary Modified Influenza B HA Polypeptides

In some embodiments, relative to a corresponding wild-type influenza B HA polypeptide, the modified influenza B HA polypeptides provided herein comprise amino acid substitutions at:
1) amino acid position 18;
2) amino acid position 28;
3) amino acid position 62;
4) amino acid position 157;
5) amino acid position 188;
6) amino acid position 121;
7) amino acid position 177;
8) amino acid position 218;
9) amino acid position 226;
10) amino acid position 257;
11) amino acid position 336;
12) amino acid position 349;
13) amino acid position 363;
14) amino acid position 366;
15) amino acid position 372;
16) amino acid position 380;
17) amino acid position 383;
18) amino acid position 388;
19) amino acid position 390;
20) amino acid position 391;
21) amino acid position 397;
22) amino acid position 399;
23) amino acid position 408;
24) amino acid position 421
25) amino acid position 430;
26) amino acid position 435;
27) amino acid position 436;
28) amino acid position 460;
29) amino acid position 467;
30) amino acid position 474;
31) amino acid position 475;
32) amino acid positions 125 and 431;
33) amino acid positions 214 and 216;
34) amino acid positions 383 and 475;
35) amino acid positions 380 and 397;
36) amino acid positions 396 and 514;
37) amino acid positions 430 and 436;
38) amino acid positions 37 and 411;
39) amino acid positions 467 and 474;
40) amino acid positions 383 and 401;
41) amino acid positions 431 and 434;
42) amino acid positions 232 and 433;
43) amino acid positions 401 and 475;
44) amino acid positions 35 and 408;
45) amino acid positions 430 and 439;
46) amino acid positions 239 and 276;
47) amino acid positions 188 and 241;
48) amino acid positions 36 and 415;
49) amino acid positions 387 and 510;
50) amino acid positions 20 and 387;
51) amino acid positions 186 and 239;
52) amino acid positions 186 and 224;
53) amino acid positions 346 and 465;
54) amino acid positions 185 and 223;
55) amino acid positions 378 and 397;
56) amino acid positions 127 and 431;
57) amino acid positions 233 and 434;
58) amino acid positions 367 and 478;
59) amino acid positions 430 and 439;
60) amino acid positions 430 and 438;
61) amino acid positions 430 and 437;
62) amino acid positions 396 and 510;
63) amino acid positions 394 and 507;
64) amino acid positions 394 and 510;
65) amino acid positions 186 and 187;
66) amino acid positions 62 and 336;
67) amino acid positions 383, 460, and 475;
68) amino acid positions 383, 401, and 475;
69) amino acid positions 28, 60, and 336;
70) amino acid positions 226, 228, and 237;
71) amino acid positions 223, 224, and 225;
72) amino acid positions 226, 237, 383, and 475;
73) amino acid positions 383, 401, 405, and 475;
74) amino acid positions 383, 401, 408, and 475;
75) amino acid positions 226, 237, 383, 460, and 475;
76) amino acid positions 226, 228, 237, 383 and 475;
77) amino acid positions 383, 401, 405, 408, and 475; or
78) amino acid positions 28, 60, 141, 143, and 336
   as indexed by reference to the amino acid sequence of SEQ ID NO: 1. As shown herein, such one or more amino acid substitution(s) can stabilize the modified influenza B HA polypeptide in a prefusion (closed) conformation, as, e.g., measured by an increased binding of the modified influenza B HA polypeptide to a stem region-specific antibody (e.g., CR9114) as compared to the corresponding wild-type influenza B HA polypeptide. In some embodiments, stabilization of the modified influenza B HA polypeptide in the prefusion conformation is measured by determining the binding ratio of a stem region-specific antibody (e.g., CR9114) to an RBS-specific antibody (e.g., R95-1D05) as compared to the corresponding wild-type influenza B HA polypeptide. In some embodiments, the binding ratio of stem region-specific antibody (e.g., CR9114) to RBS-specific antibody (e.g., R95-1D05) is at least two-fold higher for the modified influenza B HA polypeptide as compared to the corresponding wild-type influenza B HA polypeptide. In some embodiments, stabilization of the prefusion conformation is measured by determining the binding of the modified influenza B HA polypeptide to a stem region-specific antibody (e.g., CR9114) and by determining the binding ratio of a stem region-specific antibody (e.g., CR9114) to an RBS-specific antibody (e.g., R95-1D05) as compared to the corresponding wild-type influenza B HA polypeptide.

In some embodiments, the modified influenza B HA polypeptides provided herein comprise amino acid substitutions at amino acid positions 383 and 475 as indexed by reference to the amino acid sequence of SEQ ID NO: 1. In some embodiments, such amino acid substitutions are combined with one or more amino acid substitutions at amino acid position 460 (optionally in combination with amino acid substitutions at amino acid positions 226 and 227) or at amino acid positions 401 and 408 (optionally in combination with an amino acid substitution at amino acid position 405) as indexed by reference to the amino acid sequence of SEQ ID NO: 1.

In some embodiments, the modified influenza B HA polypeptides provided herein comprise amino acid substitutions at amino acid positions 383, 475, and 460 as indexed by reference to the amino acid sequence of SEQ ID NO: 1. In some embodiments, the modified influenza B HA polypeptides provided herein comprise amino acid substitutions at amino acid positions 226, 237, 383, 460, and 475 as indexed by reference to the amino acid sequence of SEQ ID NO: 1. In some embodiments, the modified influenza B HA polypeptides provided herein comprise amino acid substitutions at amino acid positions 383, 401, 405, 408, and 475 as indexed by reference to the amino acid sequence of SEQ ID NO: 1.

In some embodiments, the modified influenza B HA polypeptides provided herein comprise one or more amino acid substitutions at amino acid positions 349, 383, 397, 401, 421, 430, 436, or 460 as indexed by reference to the amino acid sequence of SEQ ID NO: 1. In some embodiments, the modified influenza B HA polypeptides provided herein comprise amino acid substitutions at amino acid position 349, 397, 421, or 430 as indexed by reference to the amino acid sequence of SEQ ID NO: 1. In some embodiments, the modified influenza B HA polypeptides provided herein comprise amino acid substitutions at amino acid positions 1) 430 and 436, 2) 383 and 401, or 3) 401 and 475 as indexed by reference to the amino acid sequence of SEQ ID NO: 1.

In some embodiments, relative to a corresponding wild-type influenza B HA polypeptide, the modified influenza B HA polypeptides provided herein comprise amino acid substitutions:
1) D18W;
2) P28T;
3) K62T;
4) Q121K;
5) S157A;
6) V177D;
7) T188N;
8) L218A;
9) K226M;
10) S257L;
11) P336T;
12) T349A;
13) T349V;
14) F363P;
15) A366P;
16) E372P;
17) A380P;
18) H383P;
19) H388W;
20) A390P;
21) H391P;
22) A397P;
23) L399P;
24) K408R;
25) K408M;
26) V421P;
27) A430P;
28) H435E;
29) H435L;
30) H435Y;
31) H435F;
32) N436P;
33) A460K;
34) A460R;
35) G467F;
36) G467Q;
37) G467Y;
38) E474Q;
39) H475Y;
40) H475W;
41) T35C and K408C;
42) G37C and K411C;
43) N232C and E433C;
44) A380C and A397C;
45) A396C and A514C;
46) H383L and H475L;
47) H383C and S401C;
48) S401C and H475C;
49) A430P and N436P;
50) M431P and L434P;
51) G467Y and E474Q;
52) Q214N and K216T;
53) L125C and M431C;
54) A430C and L439C;
55) V239C and T276C;
56) T188C and Q241C;
57) A36C and S415C;
58) S387C and L510C;
59) I20C and S387C;
60) P186C and V239C;
61) P186C and P224C;
62) A346C and N465C;
63) N185C and K223C;
64) M378C and A397C;
65) L127C and M431C;
66) G233C and L434C;
67) 1367C and A478C;
68) A430C and L439C;
69) A430C and I438C;
70) A430C and E437C;
71) A396C and L510C;
72) A394C and Q507C;
73) A394C and _L510C;
74) P186F and L187T;
75) K62T and P336T;
76) P28T, K60T, and P336T;
77) H383L, A460K, and H475L;
78) H383L, S401V, and H475L;
79) K226M, T228V, and H237L;
80) K223N, P224F, and Q225T;
81) K226Q, H237I, H383I, and H475I;
82) K226M, H237L, H383L, and H475L;
83) H383L, S4011, A4051, and H475L;
84) H383L, S401V, A405V, and H475L;
85) H383L, S401V, K408M, and H475L;
86) H383M, S401V, K408M, and H475M;
87) P28T, K60T, D141N, E143T, and P336T;
88) K226M, H237L, H383L, A460K, and H475L;
89) K226M, T228V, H237L, H383L, and H475L;
90) H383L, S401V, A405V, K408M, and H475L; or
91) H383M, S401V, A405V, K408M, and H475M,
   wherein the amino acid positions are as indexed by reference to the amino acid sequence of SEQ ID NO: 1.

In some embodiments, the modified influenza B HA polypeptides provided herein comprise amino acid substitutions H383L and H475L, or H383M and H475M, wherein the amino acid positions are as indexed by reference to the amino acid sequence of SEQ ID NO: 1. In some embodiments, such amino acid substitutions are combined with one or more additional amino acid substitutions, e.g., A460K (optionally in combination with K226M and H227L), or S401V and K408M (optionally in combination with A405V) as indexed by reference to the amino acid sequence of SEQ ID NO: 1.

In some embodiments, the modified influenza B HA polypeptides provided herein comprise amino acid substitutions H383L, H475L, and A460K, wherein the amino acid positions are as indexed by reference to the amino acid sequence of SEQ ID NO: 1. In some embodiments, the modified influenza B HA polypeptides provided herein comprise amino acid substitutions K226M, H237L, H383L, A460K, and H475L, wherein the amino acid positions are as indexed by reference to the amino acid sequence of SEQ ID NO: 1. In some embodiments, the modified influenza B HA polypeptides provided herein comprise amino acid substitutions H383M, S401V, A405V, K408M, and H475M, wherein the amino acid positions are as indexed by reference to the amino acid sequence of SEQ ID NO: 1.

In some embodiments, the modified influenza B HA polypeptides provided herein comprise the amino acid substitution T349V, A397P, V421P, or A430P as indexed by reference to the amino acid sequence of SEQ ID NO: 1. In some embodiments, the modified influenza B HA polypeptides provided herein comprise amino acid substitutions A430P and N436P, H383C and S401C, or S401C and H475C as indexed by reference to the amino acid sequence of SEQ ID NO: 1.

Exemplary influenza B HA polypeptides comprising substitutions relative to a wild-type HA polypeptide are listed below in Table 2A wherein the numbering of the amino acid positions is as indexed by reference to the amino acid sequence of SEQ ID NO: 1 and Table 2B wherein the numbering of the amino acid positions is as indexed by reference to the amino acid sequence of SEQ ID NO: 2.

TABLE 2A
Exemplary influenza B HA polypeptides (the numbering of the amino acid positions
is as indexed by reference to the amino acid sequence of SEQ ID NO: 1).

| | |
|---|---|
| A460K | K60T, P336T, P28T, D141N and E143T |
| H475L and H383L | N232C and E433C |
| H475L, H383L and A460K | N436P |
| H475L, H383L, A460K, K226M and H237L | S401C and H475C |
| H475L, H383L, K226M and H237L | T35C and K408C |
| H475L, H383L, K226M, T228V and H237L | T349V |
| H475L, H383L, S401I and A405I | V421P |
| H475L, H383L and S401V | L125C and M431C |
| H475L, H383L, S401V and A405V | A430C and L439C |
| H475L, H383L, S401V and K408M | S401C and H475C |
| H475L, H383L, S401V, K408M and A405V | H383C and S401C |
| H475M, H383M, S401V and K408M | T349A |
| H475M, H383M, S401V, K408M and A405V | P28T |
| A366P | Q214N and K216T |
| A380C and A397C | T349V |
| A380P | K60T, P28T, D141N, E143T and P336T |
| A390P | K408M |
| A396C and A514C | H475Y |
| A397P | H475W |
| A430P | H435E |
| A430P and N436P | K226M, H237L, H383L, A460K and H475L |
| E474Q | Q121K |
| F363P | K408R |
| G37C and K411C | K226M, T228V and H237L |
| G467Q | K226M |
| G467Y | H435L |
| G467Y and E474Q | L399P |
| H383C and S401C | E372P |
| H383P | A430P and N436P |
| H391P | A430P |
| K60T, P336T and P28T | A397P |

TABLE 2BA
Exemplary influenza B HA polypeptides (the numbering of the amino acid positions is as
indexed by reference to the amino acid sequence of SEQ ID NO: 2).

| | |
|---|---|
| V237C and T274C | K224M, T226V, H235L, H381L and H473L |
| T186C and Q239C | K224M, H235L, H381L and H473L K224M, |
| S413C and A34C | H235L, H381L, A458K and H473L |
| S399C and H473C | H473W |
| S385C and L508C | H433Y |
| S385C, I18C | H433F |
| P184C and V237C | H433E |
| P184C and P222C | H386W |
| N463C and A344C | H381M, S399V, A403V, K406M and H473M |
| N183C and K221C | H381M, S399V, A403V, K406M and H473M |
| M376C and A395C | H381M, S399V, K406M and H473M |
| L125C and M429C | H381L, S399V, A403V, K406M and H473L |
| H381C and S399C | H381L, S399V, K406M and H473L |
| G231C and L432C | H381L, A458K and H473L |
| A476C and I365C | T186N |
| A428C and L437C | K406R |
| A428C and I436C | D16W |
| A428C and E435C | A458R |
| A394C and L508C | A458K |
| A392C and Q505C | V419P |
| A392C and L508C | N434P |
| T347V | M429P and L432P |
| T347A | A428P and N434P |
| P334T | A428P |
| P184F and L185T | V175D |
| K221N, P222F and Q223T | S255L |
| K60T and P334T | S155A |
| K60T | L216A |
| K406M | |

A representative modified influenza B HA polypeptide according to the present disclosure may have the amino acid sequence set forth in SEQ ID NO: 3:

(SEQ ID NO: 3)
MKAIIVLLMVVTSNADRICTGITSSNSPHVVKTATQGEVNVTGVIPLT
TTPTKSHFANLKGTETRGKLCPKCLNCTDLDVALGRPKCTGKIPSARV
SILHEVRPVTSGCFPIMHDRTKIRQLPNLLRGYEHVRLSTHNVINTED
APGGPYEIGTSGSCLNITNGKGFFATMAWAVPKNKTATNPLTIEVPYI
CTEEEDQITVWGFHSDDETQMARLYGDSKPQKFTSSANGVTTHYVSQI
GGFPNQTEDGGLPQSGRIVVDYMVQKSGKTGTITYQRGILLPQKVWCA
SGKSKVIKGSLPLIGEADCLHEKYGGLNKSKPYYTGEHAKAIGNCPIW
VKTPLKLANGTKYRPPAKLLKERGFFGAIAGFLEGGWEGMIAGWHGYT
SHGAHGVAVAADLKSTQEAINKITKNLNSLSELEVKNLQRLSGPMDEL
HPEILELDEKVDDLRADTISSQIELAVLLSNEGIINSEDEHLLALERK
LKKMLGPSAVEIGNGCFETKHKCNQTCLDRIAAGTFDAGEFSLPTFDS
LNITAASLNDDGLDNHTILLYYSTAASSLAVTLMIAIFVVYMVSRDNV
SCSICL.

Another representative modified influenza B HA polypeptide according to the present disclosure may have the amino acid sequence set forth in SEQ ID NO: 5:

(SEQ ID NO: 5)
MKAIIVLLMVVTSNADRICTGITSSNSPHVVKTATQGEVNVTGVIPLT
TTPTKSHFANLKGTETRGKLCPKCLNCTDLDVALGRPKCTGKIPSARV
SILHEVRPVTSGCFPIMHDRTKIRQLPNLLRGYEHVRLSTHNVINTED
APGGPYEIGTSGSCLNITNGKGFFATMAWAVPKNKTATNPLTIEVPYI
CTEEEDQITVWGFHSDDETQMARLYGDSKPQKFTSSANGVTTHYVSQI
GGFPNQTEDGGLPQSGRIVVDYMVQKSGKTGTITYQRGILLPQKVWCA
SGKSKVIKGSLPLIGEADCLHEKYGGLNKSKPYYTGEHAKAIGNCPIW
VKTPLKLANGTKYRPPAKLLKERGFFGAIAGFLEGGWEGMIAGWMGYT
SHGAHGVAVAADLKVTQEVINMITKNLNSLSELEVKNLQRLSGAMDEL
HNEILELDEKVDDLRADTISSQIELAVLLSNEGIINSEDEMLLALERK
LKKMLGPSAVEIGNGCFETKHKCNQTCLDRIAAGTFDAGEFSLPTFDS
LNITAASLNDDGLDNHTILLYYSTAASSLAVTLMIAIFVVYMVSRDNV
SCSICL.

Accordingly, in some embodiments, the influenza B HA polypeptides of the present disclosure comprise an amino acid sequence having at least about 80%, such as at least about 85%, 90%, 91%, 92%, 93%, 94%, 95%, 96%, 97%, 98%, or 99%, including all values and subranges therebetween, sequence identity to the amino acid sequence of SEQ ID NO: 3. In some embodiments, the influenza B HA polypeptides of the present disclosure comprise an amino acid sequence having at least about 80%, such as at least about 85%, 90%, 91%, 92%, 93%, 94%, 95%, 96%, 97%, 98%, or 99%, including all values and subranges therebetween, sequence identity to the amino acid sequence of SEQ ID NO: 5. In some embodiments, the influenza B HA polypeptide comprises or consists of the amino acid sequence of SEQ ID NO: 3. In some embodiments, the influenza B HA polypeptide comprises or consists of the amino acid sequence of SEQ ID NO: 5.

Similar to wild-type influenza B HA polypeptides, the modified influenza B HA polypeptides disclosed herein are capable of forming a trimeric HA complex through symmetry operations. Thus, in some embodiments, provided herein is a trimeric influenza B HA polypeptide complex comprising three copies of any of the modified influenza B HA polypeptides according to the present disclosure. In certain embodiments, the trimeric influenza B HA polypeptide complex of the disclosure has improved stability in the prefusion conformation as compared to a trimeric influenza B HA polypeptide complex prepared from a control influenza B HA polypeptide without the one or more modifications. Conformation stability can be measured using any methods known in the art. In some embodiments, stability in the prefusion conformation is measured by an increased binding of the trimeric influenza B HA polypeptide complex of the disclosure to a stem region-specific antibody (e.g., CR9114) as compared to a trimeric influenza B HA polypeptide complex prepared from a control influenza B HA polypeptide without the one or more modifications. In some embodiments, stability in the prefusion conformation is measured by an increased binding ratio of the stem region-specific antibody (e.g., CR9114) to an RBS-specific antibody (e.g., R95-1D05) as compared to a trimeric influenza B HA polypeptide complex prepared from a control influenza B HA polypeptide without the one or more modifications.

In some embodiments, the trimeric influenza B HA polypeptide complex of the disclosure is more immunogenic as compared to a trimeric influenza B HA polypeptide complex prepared from a control influenza B HA polypeptide without the one or more modifications (e.g., a corresponding wild-type influenza B HA polypeptide without the amino acid substitutions). In some embodiments, the trimeric influenza B HA polypeptide complex of the present disclosure has a comparable immunogenicity as a trimeric influenza B HA polypeptide complex prepared from a control influenza B HA polypeptide without the one or more modifications (e.g., a corresponding wild-type influenza B HA polypeptide without the amino acid substitutions). Immunogenicity can be measured using any methods known in the art. In some embodiments, immunogenicity is measured using hemagglutination inhibition assay (HA1).

### Positional Variation of Mutations or Substitutions

While modified influenza B HA polypeptides with particular amino acid mutations or substitutions at exemplary amino acid positions are provided herein, the present disclosure also contemplates amino acid mutations or substitutions in close proximity to the specified amino acid positions. Therefore, in some embodiments, the present disclosure also comprises amino acid positions within three residues (e.g., within one or two residues) of an amino acid position specified herein. For example, the disclosure of a mutation or substitution at amino acid position 336 of influenza B HA polypeptide may also include a mutation or substitution at position 333, 334, 335, 337, 338 or 339, respectively.

This is illustrated by the exemplified modified influenza B HA polypeptides described herein, in particular with respect to mutations or substitutions resulting in disulfide bridge formation or proline stabilization. For instance, amino acid substitutions to cystine at amino acid positions 428 and 435, 428 and 436, as well as 428 and 437 all result in disulfide bridge formation. Likewise, amino acid substitutions to cystine at amino acid positions 392 and 505, 392 and 508 as well as 394 and 508 all result in disulfide bridge formation. Similarly, amino acid substitutions to proline at either amino acid position 397 or 399 stabilized the exemplified influenza B HA polypeptides in the prefusion conformation. Another example is amino acid substitutions to proline at amino acid positions 429 and 432 or, alternatively, at amino acid positions 428 and 434, which achieved the same or a similar effect.

### Nucleic Acid Construction and Expression

The present disclosure further provides artificial nucleic acid molecules encoding the disclosed modified influenza B HA polypeptides. The nucleic acids may comprise DNA or RNA and may be wholly or partially synthetic or recombinant. The modified influenza B HA polypeptides provided herein can be synthesized as DNA sequences by standard methods known in the art and subsequently cloned and expressed in a recombinant host system using a suitable vector. The modified influenza B HA polypeptides provided herein can also be synthesized as RNA, such as messenger RNA (mRNA), sequences. Reference to a nucleotide sequence as set out herein encompasses a DNA molecule with the specified sequence and encompasses an RNA molecule (e.g., mRNA) with the specified sequence in which U, or a derivative thereof, such as pseudouridine, is substituted for T, unless context requires otherwise. Other nucleotide derivatives or modified nucleotides can be incorporated into the artificial nucleic acid molecules encoding the disclosed modified influenza B HA polypeptide. The synthesized DNA or mRNA sequences encoding the modified influenza B HA polypeptides of the disclosure can be codon-optimized so that expression of the encoded protein is improved and optimized for a particular expression system. Any codon optimization algorithms known in the art can be used to generate codon-optimized nucleic acid sequences.

A representative codon-optimized mRNA sequence encoding the influenza B HA polypeptides of SEQ ID NO: 3 is set forth in SEQ ID NO: 4:

(SEQ ID NO: 4)
AUGAAAGCAAUCAUAGUGCUGCUGAUGGUGGUGACUAGCAAUGCCGAU
AGGAUCUGCACUGGAAUCACCAGCAGCAAUAGUCCCCACGUGGUGAAG
ACCGCUACCCAGGGGGAGGUGAACGUGACAGGGGUGAUCCCCCUGACC
ACCACCCCCACCAAGAGCCACUUCGCCAACCUGAAGGGCACAGAAACC
CGGGGGAAGCUGUGCCCUAAGUGCCUCAAUUGUACAGACCUCGAUGUG
GCCCUGGGGCGCCCCAAGUGCACUGGGAAGAUCCCCAGUGCACGGGUG
UCCAUCCUGCACGAGGUGCGGCCAGUGACCUCUGGCUGCUUUCCUAUC
AUGCACGAUAGGACCAAGAUCCGGCAGCUGCCCAACCUGCUGAGGGGC
UACGAGCACGUGCGCCUCAGCACUCACAACGUGAUCAAUACGGAGGAC
GCACCUGGUGGACCCUACGAGAUCGGCACCAGCGGAUCUUGUCUAAAC
AUCACCAAUGGGAAAGGGUUCUUCGCCACCAUGGCCUGGGCCGUGCCC
AAGAACAAGACCGCCACCAACCCCCUGACAAUUGAGGUGCCCUACAUC
UGCACAGAGGAAGAGGACCAGAUCACCGUCUGGGGCUUCCACUCUGAC
GACGAAACCCAGAUGGCCCGCCUGUAUGGUGACUCCAAGCCUCAGAAG
UUCACUUCUAGCGCCAAUGGAGUCACCACACACUAUGUGAGCCAGAUU
GGGGGGUUCCCCAACCAGACCGAGGAUGGGGGACUCCCCCAGUCUGGC
CGCAUCGUGGUGGACUAUAUGGUGCAAAAGAGCGGCAAGACGGGCACC
AUCACCUACCAGCGGGGCAUCCUGCUGCCCCAGAAGGUGUGGUGCGCG
UCUGGCAAAUCUAAGGUGAUCAAGGGCAGCCUCCCCCUGAUCGGGGAG
GCUGACUGCCUGCACGAGAAGUAUGGCGGACUGAACAAGUCCAAGCCA
UACUACACCGGGGAGCAUGCCAAGGCCAUUGGCAACUGCCCCAUCUGG
GUGAAGACCCCCCUGAAGCUGGCCAACGGCACCAAGUACAGGCCACCC
GCCAAGCUGCUCAAGGAGCGCGGGUUCUUCGGCGCCAUCGCCGGCUUC
CUGGAGGGAGGCUGGGAGGGCAUGAUCGCUGGGUGGCACGGGUACACC
AGCCACGGCGCCCACGGGGUGGCCGUGGCUGCUGACCUGAAGUCCACC
CAGGAGGCGAUCAACAAGAUCACCAAGAACCUGAACUCCUUGAGCGAG
CUGGAGGUGAAGAAUCUGCAGCGGCUGUCCGGCCCAAUGGACGAGCUG
CACCCAGAGAUUCUGGAGCUGGACGAGAAGGUGGACGAUCUGAGAGCU
GAUACCAUCAGCUCUCAGAUCGAGCUGGCUGUGCUCCUGAGCAACGAG
GGCAUCAUCAACAGCGAGGAUGAGCACCUCCUGGCUCUGGAGCGCAAG
CUGAAGAAGAUGCUGGGCCCCAGCGCCGUCGAGAUCGGUAACGGCUGC
UUCGAAACCAAGCACAAGUGCAACCAGACUUGUCUGGACCGGAUCGCA
GCCGGCACCUUCGACGCUGGGGAGUUCAGCCUCCCCACCUUCGACAGC
CUGAACAUCACCGCCGCCAGCCUGAACGACGACGGGCUGGACAACCAC
ACCAUCCUGCUGUACUACAGUACUGCAGCCAGCAGCCUGGCCGUGACA
CUGAUGAUCGCCAUCUUCGUGGUGUACAUGGUGUCACGGGACAAUGUG
AGCUGCAGUAUCUGCCUGUAAUAA.

A representative codon-optimized mRNA sequence encoding the influenza B HA polypeptides of SEQ ID NO: 5 is set forth in SEQ ID NO: 6:

(SEQ ID NO: 6)
AUGAAAGCAAUCAUAGUGCUGCUGAUGGUGGUGACUAGCAAUGCCGAC
AGGAUCUGCACCGGCAUCACCUCGUCCAACUCCCCCCACGUGGUGAAG
ACCGCCACGCAGGGGGAGGUGAAUGUGACGGGGGUGAUCCCCCUGACG
ACCACCCCCACCAAGUCGCACUUCGCCAACCUGAAGGGCACUGAGACG
CGCGGCAAGCUGUGCCCUAAGUGCCUCAACUGCACGGACCUGGACGUG
GCCCUGGGGAGGCCCAAGUGCACGGGCAAGAUCCCCAGCGCCAGGGUG
UCCAUCCUGCACGAGGUGAGGCCGGUCACGUCCGGGUGCUUCCCCAUC
AUGCACGACAGGACCAAGAUCCGGCAGCUGCCUAAUCUUCUGCGCGGU
UACGAGCACGUGCGCCUCAGCACGCACAACGUGAUCAACACGGAGGAU
GCCCCCGGCGGACCUUACGAGAUCGGCACCUCAGGCAGCUGCCUAAAU
AUCACCAACGGCAAAGGCUUCUUUGCCACCAUGGCAUGGGCCGUCCCA
AAGAAUAAGACGGCCACAAACCCUCUAACCAUCGAGGUGCCCUACAUC
UGCACUGAGGAAGAGGAUCAGAUAACUGUGUGGGGCUUCCACUCGGAU
GACGAAACCCAGAUGGCACGCCUGUAUGGCGAUAGUAAGCCACAGAAA
UUUACAUCAUCCGCCAAUGGCGUGACAACACAUUACGUGAGUCAGAUC
GGCGGCUUCCCCAAUCAAACAGAAGAUGGCGGCCUGCCACAGAGCGGC
CGCAUCGUCGUGGACUAUAUGGUCCAAAAAUCCGGAAAAACGGGCACC
AUCACCUAUCAGAGGGGCAUUCUGCUCCCUCAGAAGGUGUGGUGCGCC
AGUGGGAAAAGUAAAGUGAUCAAAGGCUCACUGCCUCUGAUCGGGGAG
GCCGACUGUCUGCACGAAAAGUAUGGAGGCUUGAAUAAAUCCAAGCCC
UAUUACACAGGCGAACACGCCAAGGCGAUUGGAAAUUGCCCCAUCUGG
GUGAAGACCCCACUGAAGCUGGCGAAUGGCACCAAAUAUAGGCCACCA
GCCAAGCUCCUCAAAGAGAGGGGCUUCUUUGGAGCCAUCGCCGGCUUC
UUGGAAGGCGGCUGGGAGGGCAUGAUCGCAGGUUGGAUGGGCUAUACU
AGCCAUGGCGCCCACGGCGUAGCCGUGGCCGCCGAUCUAAAAGUUACA
CAGGAGGUUAUAAAUAUGAUUACAAAAAAUCUAAACAGCCUAAGUGAA
CUGGAGGUAAAAAACCUCCAGAGGCUGUCCGGGGCAAUGGACGAGCUG
CACAACGAGAUUCUGGAACUGGACGAGAAGGUGGACGACCUUAGGGCC
GACACCAUUUCGUCACAGAUCGAGCUCGCUGUGCUGCUGUCCAAUGAG
GGCAUCAUCAAUUCAGAAGAUGAGAUGCUCCUCGCCCUGGAAAGAAAG
CUGAAAAAAAUGCUGGGCCCAUCCGCAGUGGAGAUCGGCAACGGGUGU
UUCGAAACAAAGCAUAAAUGUAACCAGACAUGUCUGGACAGAAUUGCA
GCAGGGACAUUCGAUGCCGGCGAAUUUUCGCUGCCAACCUUCGAUUCA
CUGAACAUCACUGCCGCCAGCCUCAAUGAUGAUGGGUUAGAUAACCAC
ACCAUCCUGCUGUAUUAUUCAACAGCAGCCUCGAGCCUUGCCGUGACA
CUCAUGAUCGCCAUCUUCGUGGUGUACAUGGUGUCACGUGAUAACGUG
UCGUGCUCCAUUUGUCUGUAAUAA.

In some embodiments, the artificial nucleic acid molecules (e.g., mRNAs) encoding the influenza B HA polypeptides disclosed herein comprise a nucleic acid sequence having at least about 80%, such as at least about 85%, 90%, 91%, 92%, 93%, 94%, 95%, 96%, 97%, 98%, or 99%, including all values and subranges therebetween, sequence identity to the nucleic acid sequence of SEQ ID NO: 4. In some embodiments, the artificial nucleic acid molecules (e.g., mRNAs) encoding the influenza B HA polypeptides disclosed herein comprise a nucleic acid sequence having at least about 80%, such as at least about 85%, 90%, 91%, 92%, 93%, 94%, 95%, 96%, 97%, 98%, or 99%, including all values and subranges therebetween, sequence identity to the nucleic acid sequence of SEQ ID NO: 6. In some embodiments, the artificial nucleic acid molecules (e.g., mRNAs) encoding the influenza B HA polypeptides disclosed herein comprise or consist of the nucleic acid sequence of SEQ ID NO: 4. In some embodiments, the artificial nucleic acid molecules (e.g., mRNAs) encoding the influenza B HA polypeptides disclosed herein comprise or consist of the nucleic acid sequence of SEQ ID NO: 6.

To express the modified influenza B HA polypeptides of the disclosure, suitable recombinant host cells include, but are not limited to, for example, insect cells, mammalian cells, avian cells, bacteria, and yeast cells. Examples of suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)). Examples of suitable mammalian cells include, but are not limited to, for example, Chinese hamster ovary (CHO) cells, human embryonic kidney cells (HEK293 or Expi 293 cells, typically transformed by sheared adenovirus type 5 DNA), NIH-3T3 cells, 293-T cells, Vero cells, and HeLa cells. Suitable avian cells include, but are not limited to, for example, chicken embryonic stem cells (e.g., EBx® cells), chicken embryonic fibroblasts, chicken embryonic germ cells, quail fibroblasts, and duck cells. Suitable insect cell expression systems, such as baculovirus-vectored systems, are known to those of skill in the art and described in, for instance, Summers and Smith, Texas Agricultural Experiment Station Bulletin No. 1555 (1987). Materials and methods for baculovirus/insect cell expression systems are commercially available in kit form from, for example, Invitrogen (San Diego, CA). Avian cell expression systems are also known to those of skill in the art and described in, for example, U.S. Pat. Nos. 5,340,740; 5,656,479; 5,830,510; 6,114,168; and 6,500,668. Similarly, bacterial and mammalian cell expression systems are also known in the art and described in, for example, Yeast Genetic Engineering (Barr et al., eds., 1989) Butterworths, London.

In some embodiments, the cell comprises one or more viral genes, e.g., a retinal cell that expresses a viral gene (e.g., a PER.C6™ cell). In some embodiments, the host cell is a SF9 cell of Spodoptera frugiperda. See U.S. Pat. No. 6,103,526, which is hereby incorporated by reference in its entirety. In some embodiments, the host cell is a SF9 cell of Spodoptera frugiperda which has been infected with a baculovirus vector (e.g., Autographa californica nuclear polyhedrosis virus). In some embodiments, the host cell is a CHO cell.

A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells, a suitable baculovirus expression vector, such as pFastBac (Invitrogen), is used to produce recombinant baculovirus particles. The baculovirus particles are amplified and used to infect insect cells to express recombinant protein. For expression in mammalian cells, a vector that will drive expression of the construct in the desired mammalian host cell (e.g., CHO cells) can be used.

The modified influenza B HA polypeptides can be purified using any suitable methods. For example, methods for purifying recombinant influenza HA polypeptides are known in the art. See e.g., Wang et al., Vaccine, 2006, 24(12):2176-2185. Suitable methods for purifying desired proteins including precipitation and various types of chromatography, such as hydrophobic interaction, ion exchange, affinity, chelating and size exclusion are well-known in the art. Suitable purification schemes can be created using two or more of these or other suitable methods. If desired, the modified influenza B HA polypeptides can include a “tag” that facilitates purification, such as an epitope tag or a histidine (HIS) tag. Such tagged polypeptides can conveniently be purified, for example from conditioned media, by chelating chromatography or affinity chromatography.

Purified polypeptides can be analyzed by spectroscopic methods known in the art, such as circular dichroism spectroscopy, Fourier-transform infrared spectroscopy, NMR spectroscopy, or X-ray crystallography, to investigate the presence of desired structures like helices and beta sheets. ELISA, Octet and FACS and the like can be used to investigate binding of the modified influenza B HA polypeptides of the disclosure to the broadly neutralizing antibodies known in the art, such as CR9114 (stem-specific), CR8071 (VE-specific), and R95-1D05 (RBS-specific) (Dreyfus et al., Science, 2012, 337(6100):1343-1348). Thus, modified influenza B HA polypeptides according to the disclosure having a desired conformation (e.g., stabilized prefusion conformation) can be selected.

Accordingly, in some embodiments, provided herein are artificial nucleic acids encoding any of the modified influenza B HA polypeptides described herein. The artificial nucleic acids of the disclosure can be in form of a DNA or a RNA, such as a messenger RNA (mRNA). In some embodiments, the artificial nucleic acids of the disclosure are DNA molecules. In some embodiments, the artificial nucleic acids of the disclosure are RNA molecules. In certain embodiments, the artificial nucleic acids of the disclosure are mRNA molecules.

Also provided herein are vectors comprising the artificial nucleic acid molecules (e.g., mRNAs) disclosed herein. The RNA sequences encoding a protein of interest (e.g., mRNA encoding an influenza HA protein) can be cloned into a number of types of vectors. For example, the nucleic acids can be cloned into a vector including, but not limited to, a plasmid, a phagemid, a phage derivative, an animal virus, and a cosmid. Vectors of particular interest can include expression vectors, replication vectors, probe generation vectors, sequencing vectors, and vectors optimized for in vitro transcription.

In certain embodiments, the vector can be used to express mRNA in a host cell. In various embodiments, the vector can be used as a template for in vitro transcription (IVT). The construction of optimally translated IVT mRNA suitable for therapeutic use is disclosed in detail in Sahin, et al. (2014). Nat. Rev. Drug Discov. 13, 759-780; Weissman (2015). Expert Rev. Vaccines 14, 265-281.

In some embodiments, the vectors disclosed herein can comprise at least the following, from 5′ to 3′: an RNA polymerase promoter; a polynucleotide sequence encoding a 5′ UTR; a polynucleotide sequence encoding an ORF; a polynucleotide sequence encoding a 3′ UTR; and a polynucleotide sequence encoding at least one RNA aptamer. In some embodiments, the vectors disclosed herein may comprise a polynucleotide sequence encoding a poly(A) sequence and/or a polyadenylation signal.

A variety of RNA polymerase promoters are known. In some embodiments, the promoter can be a T7 RNA polymerase promoter. Other useful promoters can include, but are not limited to, T3 and SP6 RNA polymerase promoters. Consensus nucleotide sequences for T7, T3, and SP6 promoters are known.

Also disclosed herein are host cells (e.g., mammalian cells, e.g., human cells) comprising the vectors or RNA compositions disclosed herein.

Polynucleotides can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg, Germany), cationic liposome mediated transfection using lipofection, polymer encapsulation, peptide mediated transfection, biolistic particle delivery systems such as “gene guns” (see, for example, Nishikawa, et al. (2001). Hum Gene Ther. 12(8):861-70, or the TransIT-RNA transfection Kit (Mirus, Madison, WI).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present disclosure, in order to confirm the presence of the mRNA sequence in the host cell a variety of assays may be performed.

### RNA

In certain embodiments, the vaccine or immunogenic compositions disclosed herein may comprise one or more self-amplifying ribonucleic acids encoding an influenza B HA polypeptide as disclosed herein. Antigen expression from traditional nRNA is proportional to the number of mRNA molecules successfully delivered to a subject from a vaccine or immunogenic composition. Self-amplifying RNA, however, comprise genetically-engineered replicons derived from self-replicating viruses, and therefore may be added to a vaccine or immunogenic composition in lower dosages than traditional nRNA while achieving comparable results.

In certain embodiments, the RNAs are messenger RNA s (mRNAs) comprising an open reading frame (ORF) encoding an influenza B HA polypeptide as disclosed herein. In certain embodiments, the RNAs (e.g., mRNAs) further comprises at least one 5′ UTR, 3′ UTR, poly(A) tail, and/or 5′ cap.

### A. 5′ Cap

An mRNA 5′ cap can provide resistance to nucleases found in most eukaryotic cells and promote translation efficiency. Several types of 5′ caps are known. A 7-methyiguanosine cap (also referred to as “m7G” or “Cap-0”) comprises a guanosine that is linked through a 5′-5-triphosphate bond to the first transcribed nucleotide.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp, (5′(A,G(5′)ppp(5′)A, and G(5′)ppp(5′)G. Additional cap structures are described in U.S. Publication No. US 2016/0032356 and U.S. Publication No. US 2018/0125989, which are incorporated herein by reference.

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G (the ARCA cap); G(5′)ppp(5′)A; G(5′)ppp(5′)G; m7G(5′)ppp(5′)A; m7G(5′)ppp(5′)G; m7G(5′)ppp(5′)(2′OMeA)pG; m7G(5′)ppp(5′)(2′OMeA)pU; m7G(5′)ppp(5′)(2′OMeG)pG (New England BioLabs, Ipswich, MA; TriLink Biotechnologies). 5′-capping of modified RNA may be completed post-transcriptionally using a vaccinia virus capping enzyme to generate the Cap 0 structure: m7G(5′)ppp(5′)G. Cap 1 structure may be generated using both vaccinia virus capping enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase.

In certain embodiments, the mRNA of the disclosure comprises a 5′ cap selected from the group consisting of 3′-O-Me-m7G(5′)ppp(5′)G (the ARCA cap), G(5′)ppp(5′)A, G(5′)ppp(5′)G, m7G(5′)ppp(5′)A, m7G(5′)ppp(5′)G, m7G(5′)ppp(5′)(2′OMeA)pG, m7G(5′)ppp(5′)(2′OMeA)pU, and m7G(5′)ppp(5′)(2′OMeG)pG.

In certain embodiments, the mRNA of the disclosure comprises a 5′ cap of:

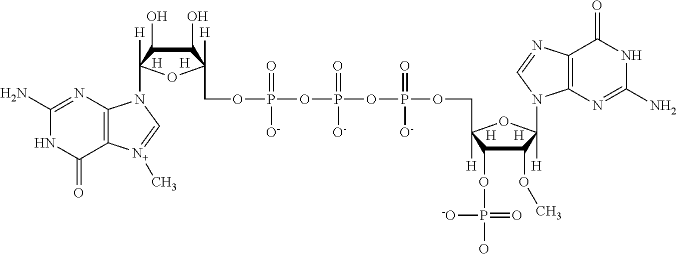

### B. Untranslated Region (UTR)

In some embodiments, the mRNA of the disclosure includes a 5′ and/or 3′ untranslated region (UTR). In mRNA, the 5′ UTR starts at the transcription start site and continues to the start codon but does not include the start codon. The 3′ UTR starts immediately following the stop codon and continues until the transcriptional termination signal.

In some embodiments, the mRNA disclosed herein may comprise a 5′ UTR that includes one or more elements that affect an mRNA's stability or translation. In some embodiments, a 5′ UTR may be about 10 to 5,000 nucleotides in length. In some embodiments, a 5′ UTR may be about 50 to 500 nucleotides in length. In some embodiments, the 5′ UTR is at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 3′ UTR comprising one or more of a polyadenylation signal, a binding site for proteins that affect an mRNA's stability of location in a cell, or one or more binding sites for miRNAs. In some embodiments, a 3′ UTR may be 50 to 5,000 nucleotides in length or longer. In some embodiments, a 3′ UTR may be 50 to 1,000 nucleotides in length or longer. In some embodiments, the 3′ UTR is at least about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Generally, these modifications improve the stability and/or pharmacokinetic properties (e.g., half-life) of the mRNA relative to their unmodified counterparts, and include, for example, modifications made to improve such mRNA resistance to in vivo nuclease digestion.

Exemplary 5′ UTRs include a sequence derived from a CMV immediate-early 1 (IE1) gene (U.S. Publication Nos. 2014/0206753 and 2015/0157565, each of which is incorporated herein by reference), or the sequence GGGAUCCUACC (SEQ ID NO: 7) (U.S. Publication No. 2016/0151409, incorporated herein by reference).

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of a hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

(SEQ ID NO: 8)
GGACAGAUCGCCUGGAGACGCCAUCCACGCUGUUUUGACCUCCAUAGA
AGACACCGGGACCGAUCCAGCCUCCGCGGCCGGGAACGGUGCAUUGGA
ACGCGGAUUCCCCGUGCCAAGAGUGACUCACCGUCCUUGACACG.

In some embodiments, the 5′ UTR comprises a nucleic acid sequence of

(SEQ ID NO: 9)
CGGGUGGCAUCCCUGUGACCCCUCCCCAGUGCCUCUCCUGGCCCUGGA
AGUUGCCACUCCAGUGCCCACCAGCCUUGUCCUAAUAAAAUUAAGUUG
CAUC.

The 5′ UTR and 3′ UTR are described in further detail in International Pub. No. WO 2012/075040, incorporated herein by reference.

### C. Polyadenylated Tail

As used herein, the terms “poly(A) sequence,” “poly(A) tail,” and “poly(A) region” refer to a sequence of adenosine nucleotides at the 3′ end of the mRNA molecule. The poly(A) tail may confer stability to the mRNA and protect it from exonuclease degradation. The poly(A) tail may enhance translation. In some embodiments, the poly(A) tail is essentially homopolymeric. For example, a poly(A) tail of 100 adenosine nucleotides may have essentially a length of 100 nucleotides. In certain embodiments, the poly(A) tail may be interrupted by at least one nucleotide different from an adenosine nucleotide (e.g., a nucleotide that is not an adenosine nucleotide). For example, a poly(A) tail of 100 adenosine nucleotides may have a length of more than 100 nucleotides (comprising 100 adenosine nucleotides and at least one nucleotide, or a stretch of nucleotides, that are different from an adenosine nucleotide). In certain embodiments, the poly(A) tail comprises the sequence:

(SEQ ID NO: 10)
AAAAAAAAAAAAAAAAAAAAAAAAAAAAAAGCAUAUGACUAAAAAAAA
AAAAAAAAAAAAAAAAAAAAAAAAAAAAAAAAAAAAAAAAAAAAAAAA
AAAAAAAAAAAAAA.

The “poly(A) tail,” as used herein, typically relates to RNA. However, in the context of the disclosure, the term likewise relates to corresponding sequences in a DNA molecule (e.g., a “poly(T) sequence”).

The poly(A) tail may comprise about 10 to about 500 adenosine nucleotides, about 10 to about 200 adenosine nucleotides, about 40 to about 200 adenosine nucleotides, or about 40 to about 150 adenosine nucleotides. The length of the poly(A) tail may be at least about 10, 50, 75, 100, 150, 200, 250, 300, 350, 400, 450, or 500 adenosine nucleotides.

In some embodiments where the nucleic acid is an RNA, the poly(A) tail of the nucleic acid is obtained from a DNA template during RNA in vitro transcription. In certain embodiments, the poly(A) tail is obtained in vitro by common methods of chemical synthesis without being transcribed from a DNA template. In various embodiments, poly(A) tails are generated by enzymatic polyadenylation of the RNA (after RNA in vitro transcription) using commercially available polyadenylation kits and corresponding protocols, or alternatively, by using immobilized poly(A) polymerases, e.g., using methods and means as described in International Pub. No. WO 2016/174271.

The nucleic acid may comprise a poly(A) tail obtained by enzymatic polyadenylation, wherein the majority of nucleic acid molecules comprise about 100 (+/−20) to about 500 (+1-50) or about 250 (+/−20) adenosine nucleotides.

In some embodiments, the nucleic acid may comprise a poly(A) tail derived from a template DNA and may additionally comprise at least one additional poly(A) tail generated by enzymatic polyadenylation, e.g., as described in International Pub. No. WO 2016/091391.

In certain embodiments, the nucleic acid comprises at least one polyadenylation signal.

In various embodiments, the nucleic acid may comprise at least one poly(C) sequence.

The term “poly(C) sequence,” as used herein, is intended to be a sequence of cytosine nucleotides of up to about 200 cytosine nucleotides. In some embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In some embodiments, the poly(C) sequence comprises about 30 cytosine nucleotides.

### D. Chemical Modification

The mRNA disclosed herein may be modified or unmodified. In some embodiments, the mRNA may comprise at least one chemical modification. In some embodiments, the mRNA disclosed herein may contain one or more modifications that typically enhance RNA stability. Exemplary modifications can include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A) and guanine (G)) or pyrimidines (thymine (T), cytosine (C), and uracil (U)). In certain embodiments, the disclosed mRNA may be synthesized from modified nucleotide analogues or derivatives of purines and pyrimidines, such as, e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, 3-D-mannosyl-queosine, phosphoramidates, phosphorothioates, peptide nucleotides, methylphosphonates, 7-deazaguanosine, 5-methylcytosine, and inosine.

In some embodiments, the disclosed mRNA may comprise at least one chemical modification including, but not limited to, pseudouridine, N1-methylpseudouridine, 2-thiouridine, 4′-thiouridine, 5-methylcytosine, 2-thio-1-methyl-1-deaza-pseudouridine, 2-thio-1-methylpseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methylpseudouridine, 4-thio-pseudouridine, 5-aza-uridine, dihydropseudouridine, 5-methyluridine, 5-methyluridine, 5-methoxyuridine, and 2′-O-methyl uridine.

In some embodiments, the chemical modification is selected from the group consisting of pseudouridine, N1-methylpseudouridine, 5-methylcytosine, 5-methoxyuridine, and a combination thereof.

In some embodiments, the chemical modification comprises N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

The preparation of such analogues is described, e.g., in U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

E. mRNA Synthesis

The mRNAs disclosed herein may be synthesized according to any of a variety of methods. For example, mRNAs according to the present disclosure may be synthesized via in vitro transcription (IVT). Some methods for in vitro transcription are described, e.g., in Geall et al. (2013) Semin. Immunol. 25(2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, an appropriate RNA polymerase (e.g., T3, T7, or SP6 RNA polymerase), DNase I, pyrophosphatase, and/or RNase inhibitor. The exact conditions may vary according to the specific application. The presence of these reagents is generally undesirable in a final mRNA product and these reagents can be considered impurities or contaminants which can be purified or removed to provide a clean and/or homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

### Immunogenic Compositions and Vaccines

The present disclosure also relates to immunogenic compositions that comprise any of the modified influenza B HA polypeptides disclosed herein or artificial nucleic acids or vectors encoding such modified influenza B HA polypeptides. As used herein, the term “immunogenic composition” refers to a composition that generates an immune response that may or may not be a protective immune response or protective immunity. The term “immune response” refers to a response of a cell of the immune system, such as a B cell, T cell, dendritic cell, macrophage or polymorphonucleocyte, to a stimulus such as an antigen, immunogen, or vaccine. An immune response can include any cell of the body involved in a host defense response, including for example, an epithelial cell that secretes an interferon or a cytokine. An immune response includes, but is not limited to, an innate and/or adaptive immune response. Methods of measuring immune responses are well known in the art and include, for example, measuring proliferation and/or activity of lymphocytes (such as B or T cells), secretion of cytokines or chemokines, inflammation, antibody production and the like. An antibody response or humoral response is an immune response in which antibodies are produced. A “cellular immune response” is one mediated by T cells and/or other white blood cells.

Also provided herein is a vaccine comprising the immunogenic composition of the present disclosure and a pharmaceutically acceptable carrier. As used herein, the term “vaccine” refers to a composition that generates a protective immune response or protective immunity in a subject. A “protective immune response” or “protective immunity” refers to an immune response that protects a subject from infection (prevents infection or prevents the development of disease associated with infection) or reduces the symptoms of infection (for instance, an infection by an influenza virus). Vaccines may elicit both prophylactic (preventative) and therapeutic responses. Methods of administration vary according to the vaccine, but may include inoculation, ingestion, inhalation or other forms of administration. Inoculations can be delivered by any of a number of routes, including parenteral, such as intravenous, subcutaneous, intraperitoneal, intradermal, intranasal, by inhalation, or intramuscular.

The term “pharmaceutically acceptable” means that the carrier, at the dosages and concentrations employed, will not cause unwanted or harmful effects in the subjects to which they are administered. Such pharmaceutically acceptable carriers and excipients are well known in the art (see e.g., Remington's Pharmaceutical Sciences, 19^{th} ed., Mack Publishing Co., Easton, PA, 1995; Pharmaceutical Formulation Development of Peptides and Proteins, S. Frokjaer and L. Hovgaard, Eds., Taylor & Francis, 2000; and Handbook of Pharmaceutical Excipients, 3^{rd} ed., A. Kibbe, Ed., Pharmaceutical Press, 2000). The term “carrier” refers to a diluent, adjuvant, excipient, or vehicle with which the composition is administered. Saline solutions and aqueous dextrose and glycerol solutions can, e.g., be employed as liquid carriers, particularly for injectable solutions. The exact formulation should suit the mode of administration. The modified influenza B HA polypeptides and/or nucleic acid (e.g., mRNA) molecules preferably are formulated and administered as a sterile solution. Sterile solutions are prepared by sterile filtration or by other methods known in the art. The solutions can then be lyophilized or filled into pharmaceutical dosage containers. The pH of the solution generally is in the range of pH 3.0 to 9.5, such as pH 5.0 to 7.5.

Accordingly, in some embodiments, provided herein is a composition comprising any of the modified influenza B HA polypeptides disclosed herein, a trimeric influenza B HA polypeptide complex comprising three copies of any of the modified influenza B HA polypeptides disclosed herein, an artificial nucleic acid encoding any of the modified influenza B HA polypeptides disclosed herein, or a vector comprising such an artificial nucleic acid. In some embodiments, provided herein is a composition comprising one or more mRNA molecules encapsulated in a lipid nanoparticle (LNP), wherein the one or more mRNA encode any of the modified influenza B HA polypeptides disclosed herein. In some embodiments, such a composition is an immunogenic composition.

In some embodiments, also provided herein is an immunogenic composition or vaccine comprising any of the modified influenza B HA polypeptides disclosed herein. In some embodiments, provided herein is an immunogenic composition or vaccine comprising a trimeric influenza B HA polypeptide complex comprising three copies of any of the modified influenza B HA polypeptides disclosed herein. In some embodiments, provided herein is an immunogenic composition or vaccine comprising an artificial nucleic acid molecule, or a vector comprising such an artificial nucleic acid molecule, that encodes any of the modified influenza B HA polypeptides disclosed herein. In some embodiments, provided herein is an immunogenic composition or vaccine comprising one or more messenger RNA (mRNA) molecules encoding any of the modified influenza B HA polypeptides disclosed herein. In certain embodiments, the one or more mRNA molecules in the immunogenic composition or vaccine of the disclosure are encapsulated in a lipid nanoparticle (LNP).

In some embodiments, the immunogenic compositions or vaccines comprise other polypeptides in addition to the modified HA B polypeptides disclosed herein. In some embodiments, the immunogenic compositions or vaccines comprise, or comprise mRNAs that encode, more than one polypeptide (e.g., two, three, four, five, six, seven, eight, nine, ten, or more polypeptides). In some embodiments, the immunogenic compositions or vaccines comprise, or comprise mRNAs that encode, three polypeptides. In some embodiments, the immunogenic compositions or vaccines comprise, or comprise mRNAs that encode, six polypeptides.

In some embodiments, the immunogenic compositions or vaccines comprise, or comprise mRNAs that encode, polypeptides derived from two or more (e.g., three, four, five, six, seven, eight, nine, or ten) influenza viral proteins selected from hemagglutinin (e.g., hemagglutinin 1 (HA1) and hemagglutinin 3 (HA3)) and neuraminidase (NA). In some embodiments, the immunogenic compositions or vaccines comprise, or comprise mRNAs that encode, one or more (e.g., three, four five, six, seven, eight, or more) polypeptides derived from an HA protein, from an NA protein, and/or from both HA and NA proteins. In some embodiments, the polypeptides are derived from different influenza strains.

In some embodiments, the immunogenic compositions or vaccines comprise, or comprise mRNAs that encode, one or more polypeptides of influenza A, B and C viruses. In some embodiments, the HA polypeptides of influenza A viruses are selected from subtypes H1, H2, H3, H4, H5, H6, H7, H8, H9, H10, H11, H12, H13, H14, H15, H16, H17, and H18. In some embodiments, the NA polypeptides of influenza A viruses are selected from subtypes N1, N2, N3, N4, N5, N6, N7, N8, N9, N10, and N11.

In some embodiments, the immunogenic compositions or vaccines comprise, or comprise mRNAs that encode, two, three, four, five, six, seven, eight, nine, or more of (i) one or more HA polypeptides, (ii) one or more NA polypeptides, or (iii) a combination of one or more HA polypeptides and NA polypeptides.

In certain embodiments, the immunogenic compositions or vaccines comprise, or comprise mRNAs that encode, two, three, four, five, six, seven, eight, nine or more of (i) one or more HA polypeptides, (ii) one or more NA polypeptides, or (iii) a combination of one or more HA polypeptides and NA polypeptides selected from H1N1, H3N2, H2N2, H5N1, H7N9, H7N7, H1N2, H9N2, H7N2, H7N3, H5N2, and H10N7 subtypes and/or B/Yamagata and B/Victoria lineages.

In some embodiments, the immunogenic composition or vaccine comprises an mRNA encoding an influenza H3 HA polypeptide, an mRNA encoding an influenza H1 HA polypeptide, and an mRNA encoding a modified influenza B HA polypeptide derived from an influenza B/Victoria lineage disclosed herein.

In some embodiments, the immunogenic composition or vaccine comprises an mRNA encoding an influenza H3 HA polypeptide, an mRNA encoding an influenza N2 NA polypeptide, an mRNA encoding an influenza H1 HA polypeptide, an mRNA encoding an influenza N1 NA polypeptide, an mRNA encoding a modified influenza B HA polypeptide derived from an influenza B/Victoria lineage disclosed herein, and an mRNA encoding an NA polypeptide from the influenza B/Victoria lineage.

In some embodiments, the immunogenic composition or vaccine comprises an mRNA encoding an influenza H3 HA polypeptide, an mRNA encoding an influenza H1 HA polypeptide, an mRNA encoding an influenza B HA polypeptide from the influenza B/Victoria lineage, and an mRNA encoding an influenza B HA polypeptide from the influenza B/Yamagata lineage, wherein at least one of the influenza B HA polypeptide from the influenza B/Victoria lineage and the influenza B HA polypeptide from the influenza B/Yamagata lineage is a modified influenza B HA polypeptide according to the present disclosure.

In some embodiments, the immunogenic composition or vaccine comprises an mRNA encoding an influenza H3 HA polypeptide, an mRNA encoding an influenza N2 NA polypeptide, an mRNA encoding an influenza H1 HA polypeptide, an mRNA encoding an influenza N1 NA polypeptide, an mRNA encoding an influenza B HA polypeptide from the influenza B/Victoria lineage, an mRNA encoding an NA polypeptide from the influenza B/Victoria lineage, an mRNA encoding an influenza B HA polypeptide from the influenza B/Yamagata lineage, and an mRNA encoding an NA polypeptide from the influenza B/Yamagata lineage, wherein at least one of the influenza B HA polypeptide from the influenza B/Victoria lineage and the influenza B HA polypeptide from the influenza B/Yamagata lineage is a modified influenza B HA polypeptide according to the present disclosure.

In some embodiments, the immunogenic composition or vaccine comprises an influenza H3 HA polypeptide, an influenza H1 HA polypeptide, and a modified influenza B HA polypeptide derived from an influenza B/Victoria lineage disclosed herein.

In some embodiments, the immunogenic composition or vaccine comprises an influenza H3 HA polypeptide, an influenza N2 NA polypeptide, an influenza H1 HA polypeptide, an influenza N1 NA polypeptide, a modified influenza B HA polypeptide derived from an influenza B/Victoria lineage disclosed herein, and an NA polypeptide from the influenza B/Victoria lineage.

In some embodiments, the immunogenic composition or vaccine comprises an influenza H3 HA polypeptide, an influenza H1 HA polypeptide, an influenza B HA polypeptide from the influenza B/Victoria lineage, and an influenza B HA polypeptide from the influenza B/Yamagata lineage, wherein at least one of the influenza B HA polypeptide from the influenza B/Victoria lineage and the influenza B HA polypeptide from the influenza B/Yamagata lineage is a modified influenza B HA polypeptide according to the present disclosure.

In some embodiments, the immunogenic composition or vaccine comprises an influenza H3 HA polypeptide, an influenza N2 NA polypeptide, an influenza H1 HA polypeptide, an influenza N1 NA polypeptide, an influenza B HA polypeptide from the influenza B/Victoria lineage, an NA polypeptide from the influenza B/Victoria lineage, an influenza B HA polypeptide from the influenza B/Yamagata lineage, and an NA polypeptide from the influenza B/Yamagata lineage, wherein at least one of the influenza B HA polypeptide from the influenza B/Victoria lineage and the influenza B HA polypeptide from the influenza B/Yamagata lineage is a modified influenza B HA polypeptide according to the present disclosure.

Each ribonucleic acid molecule may be present in the compositions disclosed herein in an amount effective to induce an immune response in a subject to which the composition is administered. In certain embodiments, each ribonucleic acid molecule may be present in the vaccine or immunogenic compositions disclosed herein in an amount ranging, for example, from about 0.1 μg to about 150 μg, such as from about 5 μg to about 120 μg, from about 10 μg to about 60 μg, or about 15 μg to about 45 μg, including all values and subranges therebetween. In certain embodiments, each ribonucleic acid molecule is present in the vaccine or immunogenic composition in an amount sufficient to encode, for example, from about 5 μg to about 120 μg, such as from about 10 μg to about 60 μg, or about 15 μg to about 45 μg of the modified influenza B HA polypeptide.

The LNP compositions of the present disclosure may be provided as a frozen liquid form or a lyophilized form. A variety of cryoprotectants may be used, including, without limitations, sucrose, trehalose, glucose, mannitol, mannose, dextrose, and the like. The cryoprotectant may constitute 5-30% (w/v) of the LNP composition. In some embodiments, the LNP composition comprises trehalose, e.g., at 5-30% (e.g., 10%) (w/v). Once formulated with the cryoprotectant, the LN P compositions may be frozen (or lyophilized and cryopreserved) at −20° C. to −80° C. The LNP compositions may be provided to a patient in an aqueous buffered solution—thawed if previously frozen, or if previously lyophilized, reconstituted in an aqueous buffered solution at bedside. The buffered solution preferably is isotonic and suitable for e.g., intramuscular or intradermal injection. In some embodiments, the buffered solution is a phosphate-buffered saline (PBS).

In some embodiments, the composition of the disclosure is an immunogenic composition capable of eliciting an immune response against influenza B viruses in a subject.

In some embodiments, the immunogenic composition or vaccine of the disclosure may further comprise one or more carriers, targeting ligands, stabilizing reagents (e.g., preservatives and antioxidants), and/or other pharmaceutically acceptable excipients to stabilize the modified influenza B HA polypeptides comprised therein, or mRNA molecules encoding the same and/or LNP encapsulating such mRNA molecules, or to facilitate administration of the immunogenic composition or vaccine. Examples of such excipients include, but are not limited to, parabens, thimerosal, thiomersal, chlorobutanol, bezalkonium chloride, and chelators (e.g., ethylenediaminetetraacetic acid, or EDTA).

### I. Lipid Nanoparticle

The term “lipid nanoparticle” or “LNP” refers to a particle having at least one dimension on the order of nanometers (e.g., 1-1,000 nm) which includes one or more lipids, for example a cationic lipid and/or non-cationic lipid, and one or more excipients selected from neutral lipids, anionic lipids, zwitterionic lipids, ionizable lipids, steroids, and polymer conjugated lipids (e.g., a pegylated lipid). Examples of suitable lipids include, but are not limited to, for example, the phosphatidyl compounds (e.g., phosphatidylglycerol, phosphatidylcholine, phosphatidylserine, phosphatidylethanolamine, sphingolipids, cerebrosides, and gangliosides). RNA-encapsulated LNP compositions are known in the art, such as those described in PCT Publication Nos. WO 2021/237084 and WO 2022/099003, the entire contents of which are incorporated by reference herein.

Any known LNP formulations may be used in the embodiments disclosed herein. In some embodiments, the LNPs comprise four categories of lipids: (i) an ionizable lipid (e.g., a cationic lipid); (ii) a PEGylated lipid; (iii) a cholesterol-based lipid, and (iv) a helper lipid.

### A. Cationic Lipid

An ionizable lipid facilitates mRNA encapsulation and may be a cationic lipid. A cationic lipid affords a positively charged environment at low pH to facilitate efficient encapsulation of the negatively charged mRNA drug substance. Exemplary cationic lipids are shown below in Table 3.

TABLE 3
Cationic lipids.

| Name | Structure |
|---|---|
| OF-02 (ML7) | 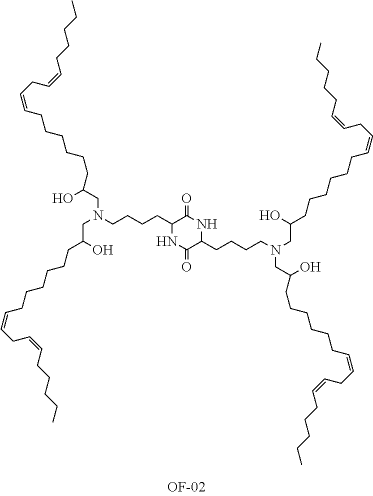 [SMILES: CCCCC/C=C\C/C=C\CCCCCCC(O)CN(CCCCC1NC(=O)C(CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC] |
| cKK-E10 | 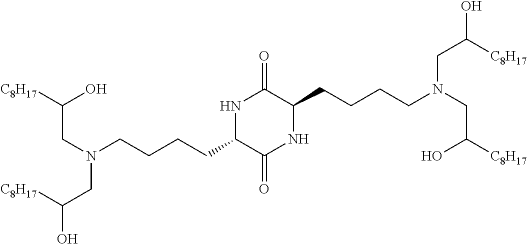 |
| GL-HEPES-E3- E10-DS-3-E18-1 ((2-(4-(2-((3- (Bis((Z)-2- hydroxyoctadec- 9-en-1- yl)amino)propyl) disulfaneyl)ethyl) piperazin-1- yl)ethyl 4-(bis(2- hydroxydecyl) amino)butanoate) | 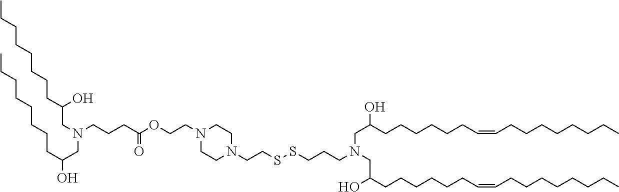 [SMILES: CCCCCCCC/C=C\CCCCCCC(O)CN(CCCSSCCN1CCN(CCOC(=O)CCCN(CC(O)CCCCCCCC)CC(O)CCCCCCCC)CC1)CC(O)CCCCCC/C=C\CCCCCCCC] |
| GL-HEPES-E3- E12-DS-4-E10 (2-(4-(2-((3- (bis(2- hydroxydecyl) amino)butyl) disulfaneyl) ethyl)piperazin- 1-yl)ethyl 4- (bis(2- hydroxydodecyl) amino)butanoate) | 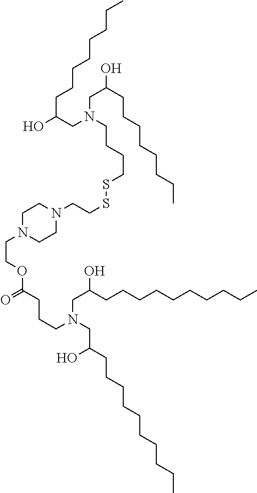 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC] |
| GL-HEPES-E3- E12-DS-3-E14 (2-(4-(2-((3- (Bis(2- hydroxytetradecyl) amino)propyl) disulfaneyl) ethyl)piperazin- 1-yl)ethyl 4- (bis(2- hydroxydodecyl) amino)butanoate) | 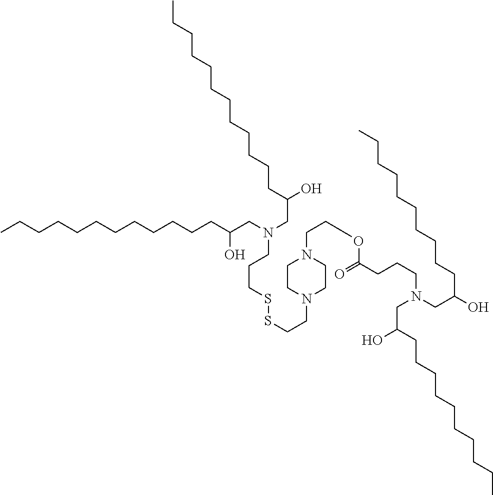 [SMILES: CCCCCCCCCCCCC(O)CN(CCCSSCCN1CCN(CCOC(=O)CCCN(CC(O)CCCCCCCCCC)CC(O)CCCCCCCCCC)CC1)CC(O)CCCCCCCCCCCC] |
| MC3 | 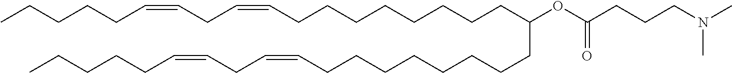 [SMILES: CCCCC/C=C\C/C=C\CCCCCCCCC(CCCCCCCC/C=C\C/C=C\CCCCC)OC(=O)CCCN(C)C] |
| SM-102 (9-heptadecanyl 8-{(2- hydroxyethyl)[6- oxo-6- (undecyloxy) hexyl]amino} octanoate) | 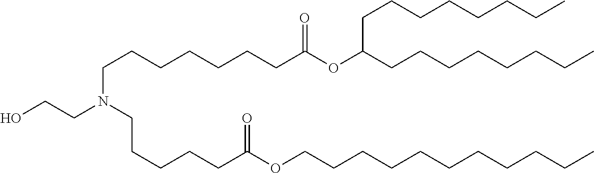 [SMILES: CCCCCCCCCCCOC(=O)CCCCCN(CCO)CCCCCCCC(=O)OC(CCCCCCCC)CCCCCCCC] |
| ALC-0315 [(4- hydroxybutyl) azanediyl]di (hexane-6,1-diyl) bis(2- hexyldecanoate) | 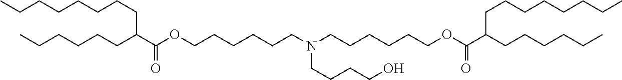 [SMILES: CCCCCCCCC(CCCCCC)C(=O)OCCCCCCN(CCCCO)CCCCCCOC(=O)C(CCCCCC)CCCCCCCC] |
| IM-001 | 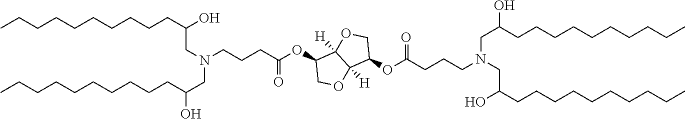 [SMILES: CCCCCCCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCC] |

The cationic lipid may be selected from the group comprising [ckkE10]/[OF-02], [(6Z,9Z,28Z,31Z)-heptatriaconta-6,9,28,31-tetraen-19-yl]4-(dimethylamino)butanoate (D-Lin-MC3-DMA); 2,2-dilinoleyl-4-dimethylaminoethyl-[1,3]-dioxolane (DLin-KC2-DMA); 1,2-dilinoleyloxy-N,N-dimethyl-3-aminopropane (DLin-DMA); di((Z)-non-2-en-1-yl)9-((4-(dimethylamino)butanoyl)oxy)heptadecanedioate (L319); 9-heptadecanyl 8-{(2-hydroxyethyl)[6-oxo-6-(undecyloxy)hexyl]amino}octanoate (SM-102); [(4-hydroxybutyl)azanediyl]di(hexane-6,1-diyl)bis(2-hexyldecanoate) (ALC-0315); [3-(dimethylamino)-2-[(Z)-octadec-9-enoyl]oxypropyl](Z)-octadec-9-enoate (DODAP); 2,5-bis(3-aminopropylamino)-N-[2-[di(heptadecyl) amino]-2-oxoethyl]pentanamide (DOGS); [(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl]N-[2-(dimethylamino)ethyl]carbamate (DC-Chol); tetrakis(8-methylnonyl) 3,3′,3″,3′″-(((methylazanediyl)bis(propane-3,1diyl))bis(azanetriyl))tetrapropionate (306Oi10); decyl(2-(dioctylammonio)ethyl)phosphate (9A1P9); ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18); bis(2-(dodecyldisulfanyl)ethyl)3,3′-((3-methyl-9-oxo-10-oxa-13,14-dithia-3,6-diazahexacosyl) azanediyl)dipropionate (BAME-016B); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200); 3,6-bis(4-(bis(2-hydroxydodecyl)amino)butyl)piperazine-2,5-dione (cKK-E12); hexa(octan-3-yl) 9,9′,9″,9′″,9″″,9′″″-((((benzene-1,3,5-tricarbonyl)yris(azanediyl))tris(propane-3,1-diyl))tris (azanetriyl))hexanonanoate (FTT5); (((3,6-dioxopiperazine-2,5-diyl)bis(butane-4,1-diyl))bis (azanetriyl))tetrakis(ethane-2,1-diyl)(9Z,9′Z,9″Z,9′″Z,12Z,12′Z,12″Z,12′″Z)-tetrakis(octadeca-9,12-dienoate) (OF-Deg-Lin); TT3; N1,N3,N5-tris(3-(didodecylamino)propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); heptadecan-9-yl 8-((2-hydroxyethyl)(8-(nonyloxy)-8-oxooctyl)amino)octanoate (Lipid 5); IM-001; and combinations thereof.

In certain embodiments, the cationic lipid is biodegradable. In various embodiments, the cationic lipid is not biodegradable. In some embodiments, the cationic lipid is cleavable. In certain embodiments, the cationic lipid is not cleavable.

Cationic lipids are described in further detail in Dong et al. (PNAS. 111(11):3955-60. 2014); Fenton et al. (Adv. Mater. 28:2939. 2016); U.S. Pat. Nos. 9,512,073; and 10,201,618, each of which is incorporated herein by reference.

### B. PEGylated Lipid

The PEGylated lipid component provides control over particle size and stability of the nanoparticle. The addition of such components may prevent complex aggregation and provide a means for increasing circulation lifetime and increasing the delivery of the lipid-nucleic acid pharmaceutical composition to target tissues (Klibanov et al., FEBS Letters 268(1):235-7. 1990). These components may be selected to rapidly exchange out of the pharmaceutical composition in vivo (see, e.g., U.S. Pat. No. 5,885,613).

Contemplated PEGylated lipids include, but are not limited to, a polyethylene glycol (PEG) chain of up to 5 kDa in length covalently attached to a lipid with alkyl chain(s) of C6-C20 (e.g., C8, C10, C12, C14, C16, or C18) length, such as a derivatized ceramide (e.g., N-octanoyl-sphingosine-1-[succinyl(methoxypolyethylene glycol)](C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); or 1,2-distearoyl-rac-glycero-polyethelene glycol (DSG-PEG), PEG-DAG; PEG-PE; PEG-S-DAG; PEG-S-DMG; PEG-cer; a PEG-dialkyoxypropylcarbamate; 2-[(polyethylene glycol)-2000]-N,N-ditetradecylacetamide (ALC-0159); and combinations thereof.

In certain embodiments, the PEG has a high molecular weight, e.g., 2000-2400 g/mol. In certain embodiments, the PEG is PEG2000 (or PEG-2K). In certain embodiments, the PEGylated lipid herein is DMG-PEG2000, DSPE-PEG2000, DLPE-PEG2000, DSG-PEG2000, C8 PEG2000, or ALC-0159 (2-[(polyethylene glycol)-2000]-N,N-ditetradecylacetamide). In certain embodiments, the PEGylated lipid herein is DMG-PEG2000.

### C. Cholesterol-Based Lipid

The cholesterol component provides stability to the lipid bilayer structure within the nanoparticle. In some embodiments, the LNPs comprise one or more cholesterol-based lipids. Suitable cholesterol-based lipids include, for example: DC-Choi (N,N-dimethyl-N-ethylcarboxamidocholesterol), 1,4-bis(3-N-oleylamino-propyl)piperazine (Gao et al., Biochem Biophys Res Comm. (1991) 179:280; Wolf et al., BioTechniques (1997) 23:139; U.S. Pat. No. 5,744,335), imidazole cholesterol ester (“ICE”; WO2011/068810), sitosterol (22,23-dihydrostigmasterol), β-sitosterol, sitostanol, fucosterol, stigmasterol (stigmasta-5,22-dien-3-ol), ergosterol; desmosterol (3β-hydroxy-5,24-cholestadiene); lanosterol (8,24-lanostadien-3b-ol); 7-dehydrocholesterol (A5,7-cholesterol); dihydrolanosterol (24,25-dihydrolanosterol); zymosterol (5α-cholesta-8,24-dien-3M-ol); lathosterol (5α-cholest-7-en-3M-ol); diosgenin ((30,25R)-spirost-5-en-3-ol); campesterol (campest-5-en-3M-ol); campestanol (5α-campestan-3b-ol); 24-methylene cholesterol (5,24(28)-cholestadien-24-methylen-3β-ol); cholesteryl margarate (cholest-5-en-3M-yl heptadecanoate); cholesteryl oleate; cholesteryl stearate and other modified forms of cholesterol. In some embodiments, the cholesterol-based lipid used in the LNPs is cholesterol.

### D. Helper Lipid

A helper lipid enhances the structural stability of the LNP and helps the LNP in endosome escape. It improves uptake and release of the mRNA drug payload. In some embodiments, the helper lipid is a zwitterionic lipid, which has fusogenic properties for enhancing uptake and release of the drug payload. Examples of helper lipids are 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), DMPC, 1,2-dilauroyl-sn-glycero-3-phosphocholine (DLPC), 1,2-Distearoylphosphatidylethanolamine (DSPE), and 1,2-dilauroyl-sn-glycero-3-phosphoethanolamine (DLPE).

Other exemplary helper lipids are dioleoylphosphatidylcholine (DOPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), phosphatidylserine, sphingolipids, sphingomyelins, ceramides, cerebrosides, gangliosides, 16-O-monomethyl PE, 16-O-dimethyl PE, 18-1-trans PE, 1-stearoyl-2-oleoyl-phosphatidyethanolamine (SOPE), or a combination thereof. In certain embodiments, the helper lipid is DOPE. In certain embodiments, the helper lipid is DSPC.

In various embodiments, the present LNPs comprise (i) a cationic lipid selected from OF-02, cKK-E10, GL-HEPES-E3-E10-DS-3-E18-1, GL-HEPES-E3-E12-DS-4-E10, or GL-HEPES-E3-E12-DS-3-E14; (ii) DMG-PEG2000; (iii) cholesterol; and (iv) DOPE.

### E. Molar Ratios of the Lipid Components

The molar ratios of the herein components are important for the LNPs' effectiveness in delivering mRNA. The molar ratio of the cationic lipid, the PEGylated lipid, the cholesterol-based lipid, and the helper lipid is A: B: C: D, where A+B+C+D=100%. In some embodiments, the molar ratio of the cationic lipid in the LNPs relative to the total lipids (i.e., A) is 35-55%, such as 35-50% (e.g., 38-42% such as 40%, or 45-50%). In some embodiments, the molar ratio of the PEGylated lipid component relative to the total lipids (i.e., B) is 0.25-2.75% (e.g., 1-2% such as 1.5%). In some embodiments, the molar ratio of the cholesterol-based lipid relative to the total lipids (i.e., C) is 20-50% (e.g., 27-30% such as 28.5%, or 38-43%). In some embodiments, the molar ratio of the helper lipid relative to the total lipids (i.e., D) is 5-35% (e.g., 28-32% such as 30%, or 8-12%, such as 10%). In some embodiments, the (PEGylated lipid+cholesterol) components have the same molar amount as the helper lipid. In some embodiments, the LNPs contain a molar ratio of the cationic lipid to the helper lipid that is more than 1.

In certain embodiments, the LNP of the disclosure comprises:
i) a cationic lipid at a molar ratio of 35% to 55% or 40% to 50% (e.g., a cationic lipid at a molar ratio of 35%, 36%, 37%, 38%, 39%, 40%, 41% 42%, 43%, 44%, 45%, 46%, 47%, 48%, 49%, 50%, 51%, 52%, 53%, 54%, or 55%);
ii) a polyethylene glycol (PEG) conjugated (PEGylated) lipid at a molar ratio of 0.25% to 2.75% or 1.00% to 2.00% (e.g., a PEGylated lipid at a molar ratio of 0.25%, 0.50%, 0.75%, 1.00%, 1.25%, 1.50%, 1.75%, 2.00%, 2.25%, 2.50%, or 2.75%);
iii) a cholesterol-based lipid at a molar ratio of 20% to 45%, 20% to 50%, 25% to 45%, or 28.5% to 43% (e.g., a cholesterol-based lipid at a molar ratio of 20%, 21%, 22%, 23%, 24%, 25%, 26%, 27%, 28%, 29%, 30%, 31%, 32%, 33%, 34%, 35%, 36%, 37%, 38%, 39%, 40%, 41% 42%, 43%, 44%, 45%, 46%, 47%, 48%, 49%, or 50%); and
iv) a helper lipid at a molar ratio of 5% to 35%, 8% to 30%, or 10% to 30% (e.g., a helper lipid at a molar ratio of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35%),
wherein all of the molar ratios are relative to the total lipid content of the LNP.

In certain embodiments, the LNP comprises: a cationic lipid at a molar ratio of 40%; a PEGylated lipid at a molar ratio of 1.5%; a cholesterol-based lipid at a molar ratio of 28.5%; and a helper lipid at a molar ratio of 30%.

In certain embodiments, the PEGylated lipid is dimyristoyl-PEG2000 (DMG-PEG2000).

In various embodiments, the cholesterol-based lipid is cholesterol.

In some embodiments, the helper lipid is 1,2-dioleoyl-SN-glycero-3-phosphoethanolamine (DOPE).

In certain embodiments, the LNP comprises: OF-02 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: cKK-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E10-DS-3-E18-1 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-4-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-3-E14 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: SM-102 at a molar ratio of 35% to 55%; DMG-PEG2000 at a molar ratio of 0.25% to 2.75%; cholesterol at a molar ratio of 20% to 50%; and DSPC at a molar ratio of 5% to 35%.

In certain embodiments, the LNP comprises: ALC-0315 at a molar ratio of 35% to 55%; ALC-0159 at a molar ratio of 0.25% to 2.75%; cholesterol at a molar ratio of 20% to 50%; and DSPC at a molar ratio of 5% to 35%.

In certain embodiments, the LNP comprises: OF-02 at a molar ratio of 40%; DMG-PEG2000 at a molar ratio of 1.5%; cholesterol at a molar ratio of 28.5%; and DOPE at a molar ratio of 30%.

In certain embodiments, the LNP comprises: cKK-E10 at a molar ratio of 40%; DMG-PEG2000 at a molar ratio of 1.5%; cholesterol at a molar ratio of 28.5%; and DOPE at a molar ratio of 30%.

In certain embodiments, the LNP comprises: GL-HEPES-E3-E10-DS-3-E18-1 at a molar ratio of 40%; DMG-PEG2000 at a molar ratio of 1.5%; cholesterol at a molar ratio of 28.5%; and DOPE at a molar ratio of 30%.

In certain embodiments, the LNP comprises: GL-HEPES-E3-E12-DS-4-E10 at a molar ratio of 40%; DMG-PEG2000 at a molar ratio of 1.5%; cholesterol at a molar ratio of 28.5%; and DOPE at a molar ratio of 30%.

In certain embodiments, the LNP comprises: GL-HEPES-E3-E12-DS-3-E14 at a molar ratio of 40%; DMG-PEG2000 at a molar ratio of 1.5%; cholesterol at a molar ratio of 28.5%; and DOPE at a molar ratio of 30%.

In certain embodiments, the LNP comprises: 9-heptadecanyl 8-{(2-hydroxyethyl)[6-oxo-6-(undecyloxy)hexyl]amino}octanoate (SM-102) at a molar ratio of 50%; 1,2-distearoyl-sn-glycero-3-phosphocholine (DSPC) at a molar ratio of 10%; cholesterol at a molar ratio of 38.5%; and 1,2-dimyristoyl-rac-glycero-3-methoxypolyethylene glycol-2000 (DMG-PEG2000) at a molar ratio of 1.5%.

In certain embodiments, the LNP comprises: (4-hydroxybutyl)azanediyl]di(hexane-6,1-diyl) bis(2-hexyldecanoate) (ALC-0315) at a molar ratio of 46.3%; 1,2-distearoyl-sn-glycero-3-phosphocholine (DSPC) at a molar ratio of 9.4%; cholesterol at a molar ratio of 42.7%; and 2-[(polyethylene glycol)-2000]-N,N-ditetradecylacetamide (ALC-0159) at a molar ratio of 1.6%.

In certain embodiments, the LNP comprises: (4-hydroxybutyl)azanediyl]di(hexane-6,1-diyl) bis(2-hexyldecanoate) (ALC-0315) at a molar ratio of 47.4%; 1,2-distearoyl-sn-glycero-3-phosphocholine (DSPC) at a molar ratio of 10%; cholesterol at a molar ratio of 40.9%; and 2-[(polyethylene glycol)-2000]-N,N-ditetradecylacetamide (ALC-0159) at a molar ratio of 1.7%.

In certain embodiments, the LNP comprises: IM-001 at a molar ratio of 40%; DMG-PEG2000 at a molar ratio of 1.5%; cholesterol at a molar ratio of 28.5%; and DOPE at a molar ratio of 30%.

To calculate the actual amount of each lipid to be put into an LNP formulation, the molar amount of the cationic lipid is first determined based on a desired N/P ratio, where N is the number of nitrogen atoms in the cationic lipid and P is the number of phosphate groups in the mRNA to be transported by the LNP. Next, the molar amount of each of the other lipids is calculated based on the molar amount of the cationic lipid and the molar ratio selected. These molar amounts are then converted to weights using the molecular weight of each lipid.

Accordingly, in some embodiments, provided herein is a composition comprising an artificial messenger RNA (mRNA) encoding any of the influenza B HA polypeptides disclosed herein encapsulated in a LNP, wherein the LNP comprises a cationic lipid. In some embodiments, the cationic lipid comprises or is OF-02. In some embodiments, the cationic lipid comprises or is cKK-E10. In some embodiments, the cationic lipid comprises or is GL-HEPES-E3-E10-DS-3-E18-1. In some embodiments, the cationic lipid comprises or is GL-HEPES-E3-E12-DS-4-E10. In some embodiments, the cationic lipid comprises or is GL-HEPES-E3-E12-DS-3-E14. In some embodiments, the cationic lipid comprises or is (4-hydroxybutyl)azanediyl]di(hexane-6,1-diyl) bis(2-hexyldecanoate) (ALC-0315). In some embodiments, the cationic lipid comprises or is IM-001.

In some embodiments, the LNP encapsulating the artificial mRNA of the present disclosure further comprises a PEGylated lipid, a cholesterol-based lipid, and a helper lipid. In some embodiments, the PEGylated lipid comprises or is DMG-PEG2000. In some embodiments, the cholesterol-based lipid comprises or is cholesterol. In some embodiments, the helper lipid comprises or is DOPE. In some embodiments, the LNP comprises the cationic lipid at a molar ratio between about 35% and about 55%, the PEGylated lipid at a molar ratio between about 0.25% and about 2.75%, the cholesterol-based lipid at a molar ratio between about 20% and about 45%, and the helper lipid at a molar ratio between about 5% and about 35%, wherein all of the molar ratios are relative to the total lipid content of the LNP. In some embodiments, the LNP comprises the cationic lipid at a molar ratio of about 40%, the PEGylated lipid at a molar ratio of about 1.5%, the cholesterol-based lipid at a molar ratio of about 28.5%, and the helper lipid at a molar ratio of about 30%, wherein all of the molar ratios are relative to the total lipid content of the LNP.

In some embodiments, provided herein is a composition comprising an artificial messenger RNA (mRNA) encoding the influenza B HA polypeptide of SEQ ID NO: 3 encapsulated in a LNP, wherein the LNP comprises GL-HEPES-E3-E12-DS-4-E10 at a molar ratio of about 40%, DMG-PEG2000 at a molar ratio of about 1.5%, cholesterol at a molar ratio of about 28.5%, and DOPE at a molar ratio of about 30%. In some embodiments, provided herein is a composition comprising an artificial messenger RNA (mRNA) encoding the influenza B HA polypeptide of SEQ ID NO: 5 encapsulated in a LNP, wherein the LNP comprises GL-HEPES-E3-E12-DS-4-E10 at a molar ratio of about 40%, DMG-PEG2000 at a molar ratio of about 1.5%, cholesterol at a molar ratio of about 28.5%, and DOPE at a molar ratio of about 30%. In some embodiments, provided herein is a composition comprising an artificial messenger RNA (mRNA) comprising the nucleic acid sequence of SEQ ID NO: 4 encapsulated in a LNP, wherein the LNP comprises GL-HEPES-E3-E12-DS-4-E10 at a molar ratio of about 40%, DMG-PEG2000 at a molar ratio of about 1.5%, cholesterol at a molar ratio of about 28.5%, and DOPE at a molar ratio of about 30%. In some embodiments, provided herein is a composition comprising an artificial messenger RNA (mRNA) comprising the nucleic acid sequence of SEQ ID NO: 6 encapsulated in a LNP, wherein the LNP comprises GL-HEPES-E3-E12-DS-4-E10 at a molar ratio of about 40%, DMG-PEG2000 at a molar ratio of about 1.5%, cholesterol at a molar ratio of about 28.5%, and DOPE at a molar ratio of about 30%.

### II. Processes for Making LNP Vaccines

The LNPs can be prepared by various techniques presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion that results in the formation of MLVs. Unilamellar vesicles (ULV) can then be formed by homogenization, sonication or extrusion of the multilamellar vesicles. In addition, unilamellar vesicles can be formed by detergent removal techniques.

Various methods are described in Patent Application Pub. Nos. US 2011/0244026, US 2016/0038432, US 2018/0153822, US 2018/0125989, and US 2021/0046192 and can be used for making LNP vaccines. One exemplary process entails encapsulating mRNA by mixing it with a mixture of lipids, without first pre-forming the lipids into lipid nanoparticles, as described in Patent Application Pub. No. US 2016/0038432. Another exemplary process entails encapsulating mRNA by mixing pre-formed LNPs with mRNA, as described in Patent Application Pub. No. US 2018/0153822.

In some embodiments, the process of preparing mRNA-loaded LNPs includes a step of heating one or more of the solutions to a temperature greater than ambient temperature, the one or more solutions being the solution comprising the pre-formed lipid nanoparticles, the solution comprising the mRNA and the mixed solution comprising the LNP-encapsulated mRNA. In some embodiments, the process includes the step of heating one or both of the mRNA solution and the pre-formed LNP solution, prior to the mixing step. In some embodiments, the process includes heating one or more of the solutions comprising the pre-formed LNPs, the solution comprising the mRNA and the solution comprising the LNP-encapsulated mRNA, during the mixing step. In some embodiments, the process includes the step of heating the LNP-encapsulated mRNA, after the mixing step. In some embodiments, the temperature to which one or more of the solutions is heated is or is greater than about 30° C., 37° C., 40° C., 45° C., 50° C., 55° C., 60° C., 65° C., or 70° C., including all values and subranges therebetween. In some embodiments, the temperature to which one or more of the solutions is heated ranges from about 25-70° C., about 30-70° C., about 35-70° C., about 40-70° C., about 45-70° C., about 50-70° C., or about 60-70° C., including all values and subranges therebetween. In some embodiments, the temperature is about 65° C.

Various methods may be used to prepare an mRNA solution suitable for the present invention. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water or a buffer at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml, including all values and subranges therebetween.

In some embodiments, an mRNA stock solution is mixed with a buffer solution using a pump. Exemplary pumps include but are not limited to gear pumps, peristaltic pumps and centrifugal pumps. Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute, including all values and subranges therebetween). In some embodiments, a buffer solution is mixed at a flow rate of, or greater than, about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute, including all values and subranges therebetween.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute, including all values and subranges therebetween). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute, including all values and subranges therebetween.

The process of incorporation of a desired mRNA into a lipid nanoparticle is referred to as “loading.” Exemplary methods are described in Lasic et al., FEBS Lett. (1992) 312:255-8. The LNP-incorporated nucleic acids may be completely or partially located in the interior space of the lipid nanoparticle, within the bilayer membrane of the lipid nanoparticle, or associated with the exterior surface of the lipid nanoparticle membrane. The incorporation of an mRNA into lipid nanoparticles is also referred to herein as “encapsulation” wherein the nucleic acid is entirely or substantially contained within the interior space of the lipid nanoparticle.

Suitable LNPs may be made in various sizes. In some embodiments, decreased size of lipid nanoparticles is associated with more efficient delivery of an mRNA. Selection of an appropriate LNP size may take into consideration the site of the target cell or tissue and to some extent the application for which the lipid nanoparticle is being made.

A variety of methods known in the art are available for sizing of a population of lipid nanoparticles. Preferred methods herein utilize Zetasizer Nano ZS (Malvern Panalytical) to measure LNP particle size. In one protocol, 10 μl of an LNP sample are mixed with 990 μl of 10% trehalose. This solution is loaded into a cuvette and then put into the Zetasizer machine. The z-average diameter (nm), or cumulants mean, is regarded as the average size for the LNPs in the sample. The Zetasizer machine can also be used to measure the polydispersity index (PDI) by using dynamic light scattering (DLS) and cumulant analysis of the autocorrelation function. Average LNP diameter may be reduced by sonication of formed LNP. Intermittent sonication cycles may be alternated with quasi-elastic light scattering (QELS) assessment to guide efficient lipid nanoparticle synthesis.

In some embodiments, the majority of purified LNPs, i.e., greater than about 50%, 55%, 60%, 65%, 70%, 75%, 80%, 85%, 90%, 95%, 96%, 97%, 98%, or 99%, including all values and subranges therebetween, of the LNPs, have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm, including all values and subranges therebetween). In some embodiments, substantially all (e.g., greater than 80 or 90%) of the purified lipid nanoparticles have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm, including all values and subranges therebetween).

In certain embodiments, the LNP has an average diameter of 30-200 nm. In various embodiments, the LNP has an average diameter of 80-150 nm.

In some embodiments, the LNPs in the present composition have an average size of less than 150 nm, less than 120 nm, less than 100 nm, less than 90 nm, less than 80 nm, less than 70 nm, less than 60 nm, less than 50 nm, less than 30 nm, or less than 20 nm, including all values and subranges therebetween.

In some embodiments, greater than about 70%, 75%, 80%, 85%, 90%, 95%, 96%, 97%, 98%, 99%, including all values and subranges therebetween, of the LNPs in the present composition have a size ranging from about 40-90 nm (e.g., about 45-85 nm, about 50-80 nm, about 55-75 nm, about 60-70 nm, including all values and subranges therebetween) or about 50-70 nm (e.g., 55-65 nm) are particular suitable for pulmonary delivery via nebulization.

In some embodiments, the dispersity, or measure of heterogeneity in size of molecules (PDI), of LNPs in a pharmaceutical composition provided by the present invention is less than about 0.5. In some embodiments, an LNP has a PDI of less than about 0.5, less than about 0.4, less than about 0.3, less than about 0.28, less than about 0.25, less than about 0.23, less than about 0.20, less than about 0.18, less than about 0.16, less than about 0.14, less than about 0.12, less than about 0.10, or less than about 0.08, including all values and subranges therebetween. The PDI may be measured by a Zetasizer machine as described above.

In some embodiments, greater than about 75%, 80%, 85%, 90%, 95%, 96%, 97%, 98%, or 99%, including all values and subranges therebetween, of the purified LNPs in a pharmaceutical composition provided herein encapsulate an mRNA within each individual particle. In some embodiments, substantially all (e.g., greater than 80% or 90%) of the purified lipid nanoparticles in a pharmaceutical composition encapsulate an mRNA within each individual particle. In some embodiments, a lipid nanoparticle has an encapsulation efficiency of 50% to 99%; or greater than about 60%, 65%, 70%, 75%, 80%, 85%, 90%, 92%, 95%, 98%, or 99%, including all values and subranges therebetween. Typically, lipid nanoparticles for use herein have an encapsulation efficiency of at least 90% (e.g., at least 91%, 92%, 93%, 94%, or 95%, including all values and subranges therebetween).

In some embodiments, an LNP has a N/P ratio of between 1 and 10. In some embodiments, a lipid nanoparticle has a N/P ratio above 1, about 1, about 2, about 3, about 4, about 5, about 6, about 7, or about 8, including all values and subranges therebetween. In further embodiments, a typical LNP herein has an N/P ratio of 4.

In some embodiments, a pharmaceutical composition according to the present invention contains at least about 0.5 g, 1 g, 5 g, 10 g, 100 g, 500 g, or 1000 g, including all values and subranges therebetween, of encapsulated mRNA. In some embodiments, a pharmaceutical composition contains about 0.1 g to 1000 g, at least about 0.5 ag, at least about 0.8 g, at least about 1 ag, at least about 5 ag, at least about 8 ag, at least about 10 ag, at least about 50 ag, at least about 100 g, at least about 500 ag, or at least about 1000 g, including all values and subranges therebetween, of encapsulated mRNA.

In some embodiments, mRNA can be made by chemical synthesis or by in vitro transcription (IVT) of a DNA template. For example, in an IVT process, a cDNA template is used to produce an mRNA transcript and the DNA template is degraded by a DNase. The transcript is purified by depth filtration and tangential flow filtration (TFF). The purified transcript is further modified by adding a cap and a tail, and the modified RNA is purified again by depth filtration and TFF.

The mRNA is then prepared in an aqueous buffer and mixed with an amphiphilic solution containing the lipid components of the LNPs. An amphiphilic solution for dissolving the four lipid components of the LNPs may be an alcohol solution. In some embodiments, the alcohol is ethanol. The aqueous buffer may be, for example, a citrate, phosphate, acetate, or succinate buffer and may have a pH of about 3.0-7.0, e.g., about 3.5, about 4.0, about 4.5, about 5.0, about 5.5, about 6.0, or about 6.5, including all values and subranges therebetween. The buffer may contain other components such as a salt (e.g., sodium, potassium, and/or calcium salts). In particular embodiments, the aqueous buffer has 1 mM citrate, 150 mM NaCl, pH 4.5.

An exemplary, nonlimiting process for making an mRNA-LNP composition involves mixing a buffered mRNA solution with a solution of lipids in ethanol in a controlled homogeneous manner, where the ratio of lipids:mRNA is maintained throughout the mixing process. In this illustrative example, the mRNA is presented in an aqueous buffer containing citric acid monohydrate, tri-sodium citrate dihydrate, and sodium chloride. The mRNA solution is added to the solution (1 mM citrate buffer, 150 mM NaCl, pH 4.5). The lipid mixture of four lipids (e.g., a cationic lipid, a PEGylated lipid, a cholesterol-based lipid, and a helper lipid) is dissolved in ethanol. The aqueous mRNA solution and the ethanol lipid solution are mixed at a volume ratio of 4:1 in a “T” mixer with a near “pulseless” pump system. The resultant mixture is then subjected for downstream purification and buffer exchange. The buffer exchange may be achieved using dialysis cassettes or a TFF system. TFF may be used to concentrate and buffer-exchange the resulting nascent LNP immediately after formation via the T-mix process. The diafiltration process is a continuous operation, keeping the volume constant by adding appropriate buffer at the same rate as the permeate flow.

### Adjuvants

In some embodiments, the immunogenic composition or vaccine of the present disclosure comprises an adjuvant. In other embodiments, the immunogenic composition or vaccine of the present disclosure does not contain an adjuvant. Similarly, in some embodiments, the immunogenic composition or vaccine of the present disclosure can be administered with an adjuvant to boost the immune response. In other embodiments, the immunogenic composition or vaccines can be administered without an adjuvant. As used herein, the term “adjuvant” refers to a substance or combination of substances that may be used to enhance an immune response to an antigen component of a vaccine or immunogenic composition. Adjuvants can include a suspension of minerals (alum, aluminum salts, including, for example, aluminum hydroxide/oxyhydroxide (AlOOH), aluminum phosphate (AlPO₄), aluminum hydroxyphosphate sulfate (AAHS) and/or potassium aluminum sulfate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as lipids and costimulatory molecules. Exemplary biological adjuvants include, but are not limited to, AS04 (Didierlaurent et al., J. Immunol., 2009, 183:6186-6197), IL-2, RANTES, GM-CSF, TNF-α, IFN-γ, G-CSF, LFA-3, CD72, B7-1, B7-2, OX-40L and 41 BBL.

In certain embodiments, the adjuvant is a squalene-based adjuvant comprising an oil-in-water adjuvant emulsion comprising at least: squalene, an aqueous solvent, a polyoxyethylene alkyl ether hydrophilic nonionic surfactant, and a hydrophobic nonionic surfactant. In certain embodiments, the emulsion is thermoreversible, optionally wherein about 90% of the population by volume of the oil drops has a size less than about 200 nm.

In certain embodiments, the polyoxyethylene alkyl ether is of formula CH₃—(CH₂)ₓ—(O—CH₂—CH₂)ₙ—OH, in which n is an integer from 10 to 60, and x is an integer from 11 to 17. In certain embodiments, the polyoxyethylene alkyl ether surfactant is polyoxyethylene(12) cetostearyl ether.

In certain embodiments, about 90% of the population by volume of the oil drops has a size less than about 160 nm. In certain embodiments, about 90% of the population by volume of the oil drops has a size less than about 150 nm. In certain embodiments, about 50% of the population by volume of the oil drops has a size less than about 100 nm. In certain embodiments, about 50% of the population by volume of the oil drops has a size less than about 90 nm.

In certain embodiments, the adjuvant further comprises at least one alditol, including, but not limited to, glycerol, erythritol, xylitol, sorbitol and mannitol.

In some embodiments the hydrophilic/lipophilic balance (HLB) of the hydrophilic nonionic surfactant is greater than or equal to about 10. In certain embodiments, the HLB of the hydrophobic nonionic surfactant is less than about 9. In certain embodiments, the HLB of the hydrophilic nonionic surfactant is greater than or equal to about 10 and the HLB of the hydrophobic nonionic surfactant is less than about 9.

In certain embodiments, the hydrophobic nonionic surfactant is a sorbitan ester, such as sorbitan monooleate, or a mannide ester surfactant. In certain embodiments, the amount of squalene is between about 5% and about 45%. In certain embodiments, the amount of polyoxyethylene alkyl ether surfactant is between about 0.9% and about 9%. In certain embodiments, the amount of hydrophobic nonionic surfactant is between about 0.7% and about 7%. In certain embodiments, the adjuvant comprises: i) about 32.5% of squalene, ii) about 6.18% of polyoxyethylene(12) cetostearyl ether, iii) about 4.82% of sorbitan monooleate, and iv) about 6% of mannitol.

In certain embodiments, the adjuvant further comprises an alkylpolyglycoside and/or a cryoprotective agent, such as a sugar, in particular dodecylmaltoside and/or sucrose.

In certain embodiments, the adjuvant comprises AF03, as described in Klucker et al., J. Pharm. Sci., 2012, 101(12):4490-4500, which is hereby incorporated by reference in its entirety. In certain embodiments, the adjuvant comprises a liposome-based adjuvant, such as SPA14. SPA14 is a liposome-based adjuvant (AS01-like) containing a toll-like receptor 4 (TLR4) agonist (E6020) and saponin (QS21).

In some embodiments, the vaccine or immunogenic composition does not comprise an adjuvant. In certain embodiments, the one or more mRNA molecules encapsulated in a LNP may serve to adjuvant one or more of the modified influenza B HA polypeptides in the vaccine or immunogenic composition. See e.g., Shirai et al., Vaccines, 2020, 8(433):1-18. In other embodiments, the vaccine or immunogenic composition further comprises an adjuvant.

### Administration

The immunogenic compositions or vaccines of the present disclosure can be formulated for administration in any way known in the art of drug delivery, for example, orally, parenterally, intravenously, intramuscularly, subcutaneously, intradermally, transdermally, intrathecally, submucosally, sublingually, rectally, vaginally, etc. In some embodiments, the immunogenic composition or vaccine of the present disclosure is formulated for sublingual administration, intramuscular administration, intradermal administration, subcutaneous administration, intravenous administration, intranasal administration, administration by inhalation, or intraperitoneal administration.

In some embodiments, the immunogenic composition or vaccine of the present disclosure is formulated for parenteral administration, such as intravenous, subcutaneous, intraperitoneal, intradermal, or intramuscular. In some embodiments, the immunogenic composition or vaccine of the present disclosure is formulated for sublingual administration. In some embodiments, the immunogenic composition or vaccine is formulated for intramuscular injection. The immunogenic composition or vaccine of the present disclosure may also be formulated for intranasal or inhalation administration. The immunogenic composition or vaccine of the present disclosure can also be formulated for any other intended route of administration.

In some embodiments, the immunogenic composition or vaccine of the present disclosure is formulated for intradermal injection, intranasal administration or intramuscular injection. General considerations in the formulation and manufacture of pharmaceutical agents for administration by these routes may be found, for example, in Remington's Pharmaceutical Sciences, 19^{th} ed., Mack Publishing Co., Easton, PA, 1995; incorporated herein by reference. At present the oral or nasal spray or aerosol route (e.g., by inhalation) are most commonly used to deliver therapeutic agents directly to the lungs and respiratory system. In some embodiments, the immunogenic composition or vaccine of the present disclosure is administered using a device that delivers a metered dosage of the vaccine or immunogenic composition. Suitable devices for use in delivering intradermal pharmaceutical compositions described herein include short needle devices such as those described in U.S. Pat. Nos. 4,886,499, 5,190,521, 5,328,483, 5,527,288, 4,270,537, 5,015,235, 5,141,496, 5,417,662, all of which are incorporated herein by reference. Intradermal compositions may also be administered by devices which limit the effective penetration length of a needle into the skin, such as those described in WO1999/34850, incorporated herein by reference, and functional equivalents thereof.

Also suitable are jet injection devices which deliver liquid vaccines or immunogenic compositions to the dermis via a liquid jet injector or via a needle which pierces the stratum corneum and produces a jet which reaches the dermis. Jet injection devices are described for example in U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, WO1997/37705, and WO1997/13537, all of which are incorporated herein by reference. Additionally, conventional syringes may be used in the classical Mantoux method of intradermal administration.

Preparations for parenteral administration typically include sterile aqueous or nonaqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like.

The immunogenic compositions or vaccines of the present disclosure may be packaged in a container, such as a prefilled syringe, a vial, or an autoinjector. In some embodiments, the immunogenic compositions or vaccines of the present disclosure are packaged in a prefilled syringe. In some embodiments, the immunogenic compositions or vaccines of the present disclosure are packaged in a vial. In some embodiments, the immunogenic compositions or vaccines of the present disclosure are packaged in an autoinjector. In other embodiments, the immunogenic compositions or vaccines of the present disclosure are packaged cartridges for patient-friendly autoinjector and infusion pump devices.

Prefilled syringes provide several advantages over other types of packages, such as convenience, affordability, accuracy, sterility, and safety. Accordingly, in some embodiments, provided herein is a pre-filled syringe comprising about 0.1, 0.15, 0.2, 0.25, 0.3, 0.35, 0.4, 0.45, or 0.5 mL volume of any of the immunogenic compositions or vaccines disclosed herein.

### Methods of Use

Also provided herein are methods of administering the immunogenic compositions or vaccines described herein to a subject. The methods may be used to vaccinate a subject to prevent an influenza B virus infection in the subject, to decrease the subject's likelihood of getting an influenza B virus infection, or to reduce the subject's likelihood of getting serious illness from an influenza B virus infection. Likewise, the present disclosure provides any of the vaccine or immunogenic compositions described herein for use in vaccinating a subject against an influenza B virus infection. Also disclosed is use of any of the immunogenic compositions as described herein for the manufacture of a vaccine for use in vaccinating a subject against an influenza B virus infection. In some embodiments, the vaccination method or use comprises administering to a subject in need thereof an immunologically effective amount of any of the immunogenic compositions or vaccines described herein.

As used herein, the term “immunologically effective amount” or “therapeutically effective amount” means an amount sufficient to immunize a subject. In some embodiments, the immunologically effective amount or therapeutically effective amount is capable of eliciting protective immunity against an infectious disease, which include, but are not limited to, an increase of antibody titers and/or T cell immunity against an infectious disease. In some embodiments, an immunologically effective amount or therapeutically effective amount of the vaccine or immunogenic composition as disclosed herein increases protective immunity in a subject by about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, about 100%, including all values and subranges therebetween, when compared with a subject who is not administered with the vaccine or immunogenic composition as disclosed herein.

Accordingly, in some embodiments, the disclosure provides a method of immunizing a subject comprising administering to the subject in need thereof any of the vaccines or immunogenic compositions described herein. In some embodiments, the disclosure provides a method of immunizing a subject comprising administering to the subject in need thereof an immunologically effective amount any of the vaccines or immunogenic compositions described herein. As used herein, “immunize” or “immunizing” means to induce in a subject a protective immune response against an influenza B virus infection. Likewise, the present disclosure provides any of the vaccine or immunogenic compositions described herein for use in immunizing a subject against an influenza B virus infection. Also disclosed is use of any of the immunogenic compositions as described herein, for the manufacture of a vaccine for use in immunizing a subject against an influenza B virus infection.

In some embodiments, the method or use prevents influenza B virus infection or disease caused by the influenza B virus infection in the subject. In some embodiments, the method or use decreases the subject's likelihood of getting an influenza B virus infection by about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, about 100%, including all values and subranges therebetween, when compared with a subject who is not administered with the vaccine or immunogenic composition as disclosed herein. In some embodiments, the method or use reduces the subject's likelihood of getting serious illness from the influenza B virus infection by about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, about 100%, including all values and subranges therebetween, when compared with a subject who is not administered with the vaccine or immunogenic composition as disclosed herein. In some embodiments, the method or use raises a protective immune response in the subject. In some embodiments, the protective immune response is an antibody response.

Also provided, in some embodiments, is a method of reducing one or more symptoms of an influenza B virus infection comprising administering to a subject in need thereof any of the vaccines or immunogenic compositions described herein. In some embodiments, provided herein is a method of reducing one or more symptoms of an influenza B virus infection comprising administering to a subject in need thereof a prophylactically effective amount of any of the vaccines or immunogenic compositions described herein.

The present disclosure provides any of the vaccine or immunogenic compositions described herein for use in reducing one or more symptoms of an influenza B virus infection. Also disclosed is any of the immunogenic compositions as described herein, for the manufacture of a vaccine for use in reducing one or more symptoms of an influenza B virus infection in a subject.

In some embodiments, the method or use of the present disclosure reduces one or more symptoms of an influenza B virus infection by about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, about 100%, including all values and subranges therebetween, when compared with a subject who is not administered with the vaccine or immunogenic composition as disclosed herein.

In some embodiments, the vaccine or immunogenic composition, and an optional adjuvant, may be administered prior to or after development of one or more symptoms of the influenza B virus infection. That is, in some embodiments, the vaccines or immunogenic compositions described herein may be administered prophylactically to prevent the influenza B virus infection or ameliorate the symptoms of a potential influenza B virus infection.

In some embodiments, the subject is at risk of infection if the subject will be in contact with other individuals or other animals known or suspected to have been infected with an influenza B virus infection and/or if the subject will be present in a location in which influenza B virus infection is known or thought to be prevalent or endemic. In some embodiments, the vaccine or immunogenic composition is administered to a subject suffering from an influenza B virus infection, or the subject is displaying one or more symptoms commonly associated with an influenza B virus infection. In some embodiments, the subject is known or believed to have been exposed to an influenza B virus infection.

Vaccines or immunogenic compositions in accordance with the present disclosure may be administered in any amount or dose appropriate to achieve a desired outcome. In some embodiments, the desired outcome is induction of a lasting adaptive immune response against the influenza B virus. In some embodiments, the desired outcome is reduction in intensity, severity, and/or frequency, and/or delay of onset of one or more symptoms associated with influenza B virus infection. In some embodiments, the desired outcome is to provide vaccines or immunogenic compositions with consistent RNA quality. The dose required may vary from subject to subject depending on the species, age, weight and general condition of the subject, the severity of the infection being treated, the particular composition being used and its mode of administration.

In some embodiments, the vaccines or immunogenic compositions described herein are administered to subjects, wherein the subjects can be any member of the animal kingdom. In some embodiments, the subject is a non-human animal. In some embodiments, the non-human subject is an avian (e.g., a chicken or a bird), a reptile, an amphibian, a fish, an insect, and/or a worm. In some embodiments, the non-human subject is a mammal (e.g., a ferret, a rodent, a mouse, a rat, a rabbit, a monkey, a dog, a cat, a sheep, cattle, a primate, and/or a pig).

In some embodiments, the vaccines or immunogenic compositions described herein are administered to a human subject. In some embodiments, a human subject is 6 months of age or older, 6 months through 35 months of age, at least two years of age, at least 3 years of age, 36 months through 8 years of age, 9 years of age or older, at least 6 months of age and less than 5 years of age, at least 6 months of age and less than 18 years of age, or at least 3 years of age and less than 18 years of age. In some embodiments, the human subject is an infant (less than 36 months). In some embodiments, the human subject is a child or adolescent (less than 18 years of age). In some embodiments, the human subject is a child of at least 6 months of age and less than 5 years of age. In some embodiments, the human subject is at least 5 years of age and less than 60 years of age. In some embodiments, the human subject is at least 5 years of age and less than 65 years of age. In some embodiments, the human subject is elderly (at least 60 years of age or at least 65 years of age). In some embodiments, the human subject is a non-elderly adult (at least 18 years of age and less than 65 years of age or at least 18 years of age and less than 60 years of age).

The methods and uses of the vaccines or immunogenic compositions described herein include administration of a single dose to a subject (i.e., no booster dose). In some embodiments, the methods and uses of the vaccines or immunogenic compositions described herein include prime-boost vaccination strategies. Prime-boost vaccination comprises administering a priming vaccine or immunogenic composition and then, after a period of time has passed, administering to the subject a boosting vaccine or immunogenic composition. The immune response is “primed” upon administration of the priming vaccine or immunogenic composition and is “boosted” upon administration of the boosting vaccine or immunogenic composition. The priming vaccine or immunogenic composition can include a vaccine or immunogenic composition as described herein and an optional adjuvant. Likewise, the boosting vaccine or immunogenic composition can include a vaccine or immunogenic composition as described herein and an optional adjuvant. The priming vaccine or immunogenic composition can be, but need not be, the same as the boosting vaccine or immunogenic composition. Administration of the boosting vaccine or immunogenic composition is generally weeks or months after administration of the priming vaccine or immunogenic composition, preferably about 2-3 weeks or 4 weeks, or 8 weeks, or 16 weeks, or 20 weeks, or 24 weeks, or 28 weeks, or 32 weeks. In certain embodiments, the recipient of the prime-boost vaccination is a naïve subject, typically a naïve infant or child.

The vaccine or immunogenic composition can be administered using any suitable route of administration, including, for example, parenteral delivery, as discussed above. In some embodiments, the vaccine or immunogenic composition is administered intramuscularly, intradermally, subcutaneously, intravenously, intranasally, by inhalation, or intraperitoneally.

### Other Applications

The modified influenza B HA polypeptides disclosed herein may have other applications, such as use in an in vitro method for preparation of a trimeric influenza B HA polypeptide complex. Accordingly, provided herein is an in vitro method of preparing a trimeric influenza B HA polypeptide complex, the method comprising expressing an artificial nucleic acid molecule encoding any of the modified influenza B HA polypeptides disclosed herein in a host cell to prepare the trimeric influenza B HA polypeptide complex. In some embodiments, the artificial nucleic acid molecule encoding the modified influenza B HA polypeptide is part of a vector. In other embodiments, the expression of the modified influenza B HA polypeptide is by culturing the host cell in cell culture medium. Thus, in some embodiments, provided herein is an in vitro method of preparing a trimeric influenza B HA polypeptide complex, the method comprising culturing the host cell in a cell culture medium, and expressing the trimeric influenza B HA polypeptide complex. In some embodiments, the in vitro method disclosed herein further comprises a step of purifying the trimeric influenza B HA polypeptide complex from the cell culture medium.

In certain embodiments, the trimeric influenza B HA polypeptide complex prepared according to the in vitro method disclosed herein is more stable in prefusion conformation as compared to a trimeric influenza B HA polypeptide complex prepared from a control influenza B HA polypeptide without the one or more modifications. In some embodiments, stability in prefusion conformation is measured by an increased binding of the trimeric influenza B HA polypeptide complex to a stem region-specific antibody (e.g., CR9114) as compared to a trimeric influenza B HA polypeptide complex prepared from a control influenza B HA polypeptide without the one or more modifications. In certain embodiments, stability in prefusion conformation is measured by an increased binding ratio of a stem region-specific antibody (e.g., CR9114) to an RBS-specific antibody (e.g., R95-1D05) as compared to a trimeric influenza B HA polypeptide complex prepared from a control influenza B HA polypeptide without the one or more modifications. In some embodiments, stability in prefusion conformation is measured by an increased binding of the trimeric influenza B HA polypeptide complex to a stem region-specific antibody (e.g., CR9114) and an increased binding ratio of a stem region-specific antibody (e.g., CR9114) to an RBS-specific antibody (e.g., R95-1D05) as compared to a trimeric influenza B HA polypeptide complex prepared from a control influenza B HA polypeptide without the one or more modifications.

In certain embodiments, the trimeric influenza B HA polypeptide complex prepared according to the in vitro method disclosed herein is more immunogenic as compared to a trimeric influenza B HA polypeptide complex prepared from a control influenza B HA polypeptide without the one or more modifications. Immunogenicity can be measured using any methods known in the art. For instance, in some embodiments, immunogenicity is measured using hemagglutination inhibition assay (HAI).

### FIRST SET OF REPRESENTATIVE EMBODIMENTS OF THE PRESENT DISCLOSURE

Embodiment 1. A modified influenza B hemagglutinin (HA) polypeptide comprising a head region and a stem region, wherein the modified influenza B HA polypeptide comprises one or more modifications selected from:
a) at least one proline mutation in the stem region, wherein the at least one proline mutation breaks at least one helical structure in the stem region of the modified influenza B HA polypeptide in a postfusion conformation;
b) at least two cysteine mutations, wherein the at least two cysteine mutations form a disulfide bridge in the modified influenza B HA polypeptide;
c) one or more amino acid mutations in the head and/or stem region, wherein the one or more amino acid mutations stabilize the modified influenza B HA polypeptide in a prefusion conformation through interface stabilization;
d) one or more amino acid mutations in the head and/or stem region, wherein the one or more amino acid mutations inactivate one or more pH sensors in the head and/or stem region;
e) at least one amino acid mutation in the head region, wherein the at least one amino acid mutation introduces at least one N-linked glycosylation motif in the head region;
f) at least one amino acid mutation in the stem region, wherein the at least one amino acid mutation introduces or disrupts a N-linked glycosylation motif in the stem region; and
g) at least one amino acid mutation in the head region, wherein the at least one amino acid mutation reduces sialic acid binding of the modified influenza B HA polypeptide as compared to a control influenza B HA polypeptide without the at least one amino acid mutation that reduces sialic acid binding of the modified influenza B HA polypeptide.

Embodiment 2. The modified influenza B HA polypeptide of Embodiment 1, comprising at least one of two of the modifications listed in a) through g).

Embodiment 3. The modified influenza B HA polypeptide of Embodiment 1 or 2, comprising at least one of three of the modifications listed in a) through g).

Embodiment 4. The modified influenza B HA polypeptide of any one of Embodiments 1-3, comprising at least one of four of the modifications listed in a) through g).

Embodiment 5. The modified influenza B HA polypeptide of any one of Embodiments 1-4, comprising at least one of five of the modifications listed in a) through g).

Embodiment 6. The modified influenza B HA polypeptide of any one of Embodiments 1-5, comprising at least one of six of the modifications listed in a) through g).

Embodiment 7. The modified influenza B HA polypeptide of any one of Embodiments 1-6, comprising at least one of each of the modifications listed in a) through g).

Embodiment 8. The modified influenza B HA polypeptide of any one of Embodiments 1-7, wherein the one or more modifications stabilize the modified influenza B HA polypeptide in a prefusion conformation.

Embodiment 9. The modified influenza B HA polypeptide of Embodiment 8, wherein stabilization of the prefusion conformation is measured by an increased binding of the modified influenza B HA polypeptide to a stem region-specific antibody as compared to a wild-type influenza B HA polypeptide and/or an increased binding ratio of the stem region-specific antibody to a RBS-specific antibody as compared to the wild type influenza B HA polypeptide.

Embodiment 10. The modified influenza B HA polypeptide of any one of Embodiments 1-9, wherein the head region is a segment of the modified influenza B HA polypeptide at approximately amino acid positions 57-307 as indexed by reference to the amino acid sequence of SEQ ID NO: 1.

Embodiment 11. The modified influenza B HA polypeptide of any one of Embodiments 1-10, wherein the stem region is a segment of the modified influenza B HA polypeptide at approximately amino acid positions 16-56 and 308-547 as indexed by reference to the amino acid sequence of SEQ ID NO: 1.

Embodiment 12. The modified influenza B HA polypeptide of any one of Embodiments 1-11, wherein the at least one proline mutation is at amino acid position 363, 366, 371, 372, 376, 380, 383, 390, 391, 393, 395, 397, 399, 421, 430, 431, 434, 436, and/or 490 as indexed by reference to the amino acid sequence of SEQ ID NO: 1.

Embodiment 13. The modified influenza B HA polypeptide of Embodiment 12, wherein the at least one proline mutation is at amino acid position 372, 397, 399, 421, 430, 431, 434, and/or 436.

Embodiment 14. The modified influenza B HA polypeptide of any one of Embodiments 1-13, wherein the at least two cysteine mutations are at amino acid positions 20 and 387, 35 and 408, 36 and 415,37 and 411, 125 and 431, 127 and 431, 185 and 223, 186 and 224, 186 and 239, 188 and 241, 232 and 433, 233 and 434, 239 and 276, 346 and 465, 367 and 478, 378 and 397, 380 and 397, 383 and 401, 387 and 510, 394 and 507, 394 and 510, 396 and 510, 396 and 514, 401 and 475, 430 and 437, 430 and 438, and/or 430 and 439, as indexed by reference to the amino acid sequence of SEQ ID NO: 1.

Embodiment 15. The modified influenza B HA polypeptide of Embodiment 14, wherein the at least two cysteine mutations are at amino acid positions 20 and 387, 36 and 415, 125 and 431, 127 and 431, 185 and 223, 186 and 224, 186 and 239, 188 and 241, 233 and 434, 239 and 276, 346 and 465, 367 and 478, 378 and 397, 383 and 401, 387 and 510, 394 and 507, 394 and 510, 396 and 510, 401 and 475, 430 and 437, 430 and 438, and/or 430 and 439.

Embodiment 16. The modified influenza B HA polypeptide of any one of Embodiments 1-15, wherein the one or more amino acid mutations that stabilize the modified influenza B HA polypeptide in the prefusion conformation through interface stabilization comprise at least one cavity filling mutation in the stem region and/or one or more amino acid mutations in the head and/or stem region to form a polar interaction with a neighboring amino acid residue.

Embodiment 17. The modified influenza B HA polypeptide of Embodiment 16, wherein the at least one cavity filling mutation is at amino acid position 460, 467, and/or 474 as indexed by reference to the amino acid sequence of SEQ ID NO: 1.

Embodiment 18. The modified influenza B HA polypeptide of Embodiment 17, wherein the at least one cavity filling mutation is a substitution at amino acid position 460 with a Lysine (K), at amino acid position 467 with a Phenylalanine (F) or a Tyrosine (Y), and/or at amino acid position 474 with a Glutamine (Q).

Embodiment 19. The modified influenza B HA polypeptide of Embodiment 16, wherein the polar interaction comprises a salt bridge or a hydrogen bond.

Embodiment 20. The modified influenza B HA polypeptide of Embodiments 1-19, wherein the one or more amino acid mutations that stabilize the modified influenza B HA polypeptide in the prefusion conformation through interface stabilization are at amino acid positions 18, 121, 188, 226, 228, 408, 435, and/or 460 as indexed by reference to the amino acid sequence of SEQ ID NO: 1.

Embodiment 21. The modified influenza B HA polypeptide of any one of Embodiments 1-20, wherein the one or more amino acid mutations that inactivate one or more pH sensors in the head and/or stem region are at amino acid position 226, 228, 237, 239, 383, 388, 391, 401, 405, 408, 435, 460, 474 and/or 475 as indexed by reference to the amino acid sequence of SEQ ID NO: 1.

Embodiment 22. The modified influenza B HA polypeptide of Embodiment 21, wherein the one or more amino acid mutations that inactivate one or more pH sensors in the head and/or stem region are at amino acid positions 226, 228, 237, 239, 383, 388, 401, 405, 408, 435, 460, and/or 475.

Embodiment 23. The modified influenza B HA polypeptide of any one of Embodiments 1-22, wherein the head region comprises a receptor binding site (RBS), and wherein the at least one N-linked glycosylation motif introduced in the head region is located in or adjacent to the RBS of the head region.

Embodiment 24. The modified influenza B HA polypeptide of Embodiment 23, wherein the RBS is a region of the modified influenza B HA polypeptide made up of residues at amino acid positions 110, 151-157, 165, 173-175, 208-218, 248, and 254-259 as indexed by reference to the amino acid sequence of SEQ ID NO: 1.

Embodiment 25. The modified influenza B HA polypeptide of any one of Embodiments 23 or 24, wherein the at least one N-linked glycosylation motif introduced in the head region is generated by introducing at least one substitution at amino acid position 60, 62, 141, 143, 186, 187, 214, 216, 223, and/or 224 as indexed by reference to the amino acid sequence of SEQ ID NO: 1.

Embodiment 26. The modified influenza B HA polypeptide of any one of Embodiments 1-25, wherein the at least one N-linked glycosylation motif introduced or disrupted in the stem region is generated by introducing at least one substitution at amino acid position 28, 336, and/or 349 as indexed by reference to the amino acid sequence of SEQ ID NO: 1.

Embodiment 27. The modified influenza B HA polypeptide of any one of Embodiments 1-26, wherein the N-linked glycosylation motif comprises a consensus sequence of NxS/Ty, wherein x and y are not proline (P).

Embodiment 28. The modified influenza B HA polypeptide of any one of Embodiments 1-27, wherein the at least one amino acid mutation that reduces sialic acid binding of the modified influenza B HA polypeptide is at amino acid position 157, 177, 218, and/or 257 as indexed by reference to the amino acid sequence of SEQ ID NO: 1.

Embodiment 29. A modified influenza B HA polypeptide comprising amino acid substitutions at:
a) amino acid positions 383, 475, and 460;
b) amino acid positions 226, 237, 383, 460, and 475; or
c) amino acid positions 383, 401, 405, 408, and 475 as indexed by reference to the amino acid sequence of SEQ ID NO: 1.

Embodiment 30. The modified influenza B HA polypeptide of Embodiment 29, comprising amino acid substitutions:
a) H383L, H475L, and A460K;
b) K226M, H237L, H383L, A460K, and H475L; or
c) H383M, S401V, A405V, K408M, and H475M.

Embodiment 31. The modified influenza B HA polypeptide of any one of Embodiments 1-30, wherein the modified influenza B HA polypeptide is from a B/Yamagata influenza virus.

Embodiment 32. The modified influenza B HA polypeptide of Embodiment 31, wherein the B/Yamagata influenza virus is B/Phuket/3073/2013.

Embodiment 33. The modified influenza B HA polypeptide of any one of Embodiments 1-30, wherein the modified influenza B HA polypeptide is from a B/Victoria influenza virus.

Embodiment 34. The modified influenza B HA polypeptide of Embodiment 33, wherein the B/Victoria influenza virus is B/Austria/1359417/2021.

Embodiment 35. The modified influenza B HA polypeptide of any one of Embodiments 1-34, further comprising a signal peptide.

Embodiment 36. The modified influenza B HA polypeptide of Embodiment 35, wherein the signal peptide is an influenza HA signal peptide.

Embodiment 37. A trimeric influenza B HA polypeptide complex, comprising three copies of the modified influenza B HA polypeptide of any one of Embodiments 1-36.

Embodiment 38. The trimeric influenza B HA polypeptide complex of Embodiment 37, wherein the trimeric influenza B HA polypeptide complex is more stable in a prefusion conformation as compared to a trimeric influenza B HA polypeptide complex prepared from a control influenza B HA polypeptide without the one or more modifications.

Embodiment 39. The trimeric influenza B HA polypeptide complex of Embodiment 38, wherein stability in prefusion conformation is measured by an increased binding of the trimeric influenza B HA polypeptide complex to a stem region-specific antibody as compared to the trimeric influenza B HA polypeptide complex prepared from the control influenza B HA polypeptide without the one or more modifications and/or an increased binding ratio of the stem region-specific antibody to a RBS-specific antibody as compared to the trimeric influenza B HA polypeptide complex prepared from the control influenza B HA polypeptide without the one or more modifications.

Embodiment 40. The trimeric influenza B HA polypeptide complex of any one of Embodiments 37-40, wherein the trimeric influenza B HA polypeptide complex is more immunogenic as compared to a trimeric influenza B HA polypeptide complex prepared from a control influenza B HA polypeptide without the one or more modifications.

Embodiment 41. The trimeric influenza B HA polypeptide complex of Embodiment 40, wherein immunogenicity is measured using a hemagglutination-inhibition assay.

Embodiment 42. An artificial nucleic acid encoding the modified influenza B HA polypeptide of any one of Embodiments 1-36.

Embodiment 43. A vector comprising the artificial nucleic acid of Embodiment 42.

Embodiment 44. A host cell comprising the vector of Embodiment 43.

Embodiment 45. A composition comprising the modified influenza B HA polypeptide of any one of Embodiments 1-36, the trimeric influenza B HA polypeptide complex of any one of Embodiments 37-41, the artificial nucleic acid of Embodiment 42, or the vector of Embodiment 43.

Embodiment 46. A composition comprising one or more messenger RNA (mRNA) molecules encapsulated in a lipid nanoparticle (LNP), wherein the one or more mRNA encode the modified influenza B HA polypeptide of any one of Embodiments 1-36.

Embodiment 47. The composition of Embodiment 45 or 46, wherein the composition is an immunogenic composition.

Embodiment 48. A vaccine comprising the composition of Embodiment 47, and a pharmaceutically acceptable carrier.

Embodiment 49. The vaccine of Embodiment 48, further comprising an adjuvant.

Embodiment 50. A method of immunizing a subject, the method comprising administering to the subject in need thereof the vaccine of Embodiment 48 or 49.

Embodiment 51. The method of Embodiment 50, wherein the method prevents an influenza B virus infection in the subject, decreases the subject's likelihood of getting an influenza B virus infection, or reduces the subject's likelihood of getting serious illness from an influenza B virus infection.

Embodiment 52. The method of Embodiment 50 or 51, wherein the subject is a human.

Embodiment 53. The method of Embodiment 52, wherein the human is 6 months of age or older, less than 18 years of age, at least 6 months of age and less than 18 years of age, at least 18 years of age and less than 65 years of age, at least 6 months of age and less than 5 years of age, at least 5 years of age and less than 65 years of age, at least 60 years of age, or at least 65 years of age.

Embodiment 54. The method of any one of Embodiments 50-53, wherein the vaccine is administered intramuscularly, intradermally, subcutaneously, intravenously, intranasally, by inhalation, or intraperitoneally.

Embodiment 55. A method of reducing one or more symptoms of an influenza B virus infection, the method comprising administering to a subject in need thereof the vaccine of Embodiment 48 or 49.

Embodiment 56. An in vitro method of preparing a trimeric influenza B HA polypeptide complex, the method comprising culturing the host cell of Embodiment 44 in a cell culture medium, and expressing the trimeric influenza B HA polypeptide complex.

Embodiment 57. The in vitro method of Embodiment 56, wherein the trimeric influenza B HA polypeptide complex is more stable in prefusion conformation as compared to a trimeric influenza B HA polypeptide complex prepared from a control influenza B HA polypeptide without the one or more modifications.

Embodiment 58. The in vitro method of Embodiment 57, wherein stability in prefusion conformation is measured by an increased binding of the trimeric influenza B HA polypeptide complex to a stem region-specific antibody as compared to the trimeric influenza B HA polypeptide complex prepared from the control influenza B HA polypeptide without the one or more modifications and/or an increased binding ratio of the stem region-specific antibody to a RBS-specific antibody as compared to the trimeric influenza B HA polypeptide complex prepared from the control influenza B HA polypeptide without the one or more modifications.

Embodiment 59. The in vitro method of any one of Embodiments 56-58, wherein the trimeric influenza B HA polypeptide complex is more immunogenic as compared to a trimeric influenza B HA polypeptide complex prepared from a control influenza B HA polypeptide without the one or more modifications.

Embodiment 60. The in vitro method of Embodiment 59, wherein, wherein immunogenicity is measured using a hemagglutination-inhibition assay.

Embodiment 61. The in vitro method of any one of Embodiments 56-60, further comprising a step of purifying the trimeric influenza B HA polypeptide complex from the cell culture medium.

### SECOND SET OF REPRESENTATIVE EMBODIMENTS OF THE PRESENT DISCLOSURE

Embodiment 1. A modified influenza B hemagglutinin (HA) polypeptide comprising a head region and a stem region, wherein the modified influenza B HA polypeptide comprises one or more modifications selected from:
a) at least one proline mutation in the stem region, wherein the at least one proline mutation breaks at least one helical structure in the stem region of the modified influenza B HA polypeptide in a postfusion conformation;
b) at least two cysteine mutations, wherein the at least two cysteine mutations form a disulfide bridge in the modified influenza B HA polypeptide;
c) one or more amino acid mutations in the head and/or stem region, wherein the one or more amino acid mutations stabilize the modified influenza B HA polypeptide in a prefusion conformation through interface stabilization;
d) one or more amino acid mutations in the head and/or stem region, wherein the one or more amino acid mutations inactivate one or more pH sensors in the head and/or stem region;
e) at least one amino acid mutation in the head region, wherein the at least one amino acid mutation introduces at least one N-linked glycosylation motif in the head region;
f) at least one amino acid mutation in the stem region, wherein the at least one amino acid mutation introduces or disrupts a N-linked glycosylation motif in the stem region; and
g) at least one amino acid mutation in the head region, wherein the at least one amino acid mutation reduces sialic acid binding of the modified influenza B HA polypeptide as compared to a control influenza B HA polypeptide without the at least one amino acid mutation that reduces sialic acid binding of the modified influenza B HA polypeptide.

Embodiment 2. The modified influenza B HA polypeptide of Embodiment 1, comprising at least one of two, three, four, five, six or each of the modifications listed in a) through g).

Embodiment 3. The modified influenza B HA polypeptide of Embodiment 1 or 2, wherein the one or more modifications stabilize the modified influenza B HA polypeptide in a prefusion conformation, optionally wherein stabilization of the prefusion conformation is measured by an increased binding of the modified influenza B HA polypeptide to a stem region-specific antibody as compared to a wild-type influenza B HA polypeptide and/or an increased binding ratio of the stem region-specific antibody to an RBS-specific antibody as compared to the wild type influenza B HA polypeptide.

Embodiment 4. The modified influenza B HA polypeptide of any one of Embodiments 1-3, wherein:
(i) the head region is a segment of the modified influenza B HA polypeptide at approximately amino acid positions 57-307 as indexed by reference to the amino acid sequence of SEQ ID NO: 1; and/or
(ii) the stem region is a segment of the modified influenza B HA polypeptide at approximately amino acid positions 16-56 and 308-547 as indexed by reference to the amino acid sequence of SEQ ID NO: 1.

Embodiment 5. The modified influenza B HA polypeptide of any one of Embodiments 1-4, wherein:
(i) the at least one proline mutation is at amino acid position 363, 366, 371, 372, 376, 380, 383, 390, 391, 393, 395, 397, 399, 421, 430, 431, 434, 436, and/or 490 as indexed by reference to the amino acid sequence of SEQ ID NO: 1; and/or
(ii) the at least two cysteine mutations are at amino acid positions 20 and 387, 35 and 408, 36 and 415, 37 and 411, 125 and 431, 127 and 431, 185 and 223, 186 and 224, 186 and 239, 188 and 241, 232 and 433, 233 and 434, 239 and 276, 346 and 465, 367 and 478, 378 and 397, 380 and 397, 383 and 401, 387 and 510, 394 and 507, 394 and 510, 396 and 510, 396 and 514, 401 and 475, 430 and 437, 430 and 438, and/or 430 and 439, as indexed by reference to the amino acid sequence of SEQ ID NO: 1.

Embodiment 6. The modified influenza B HA polypeptide of any one of Embodiments 1-5, wherein the one or more amino acid mutations that stabilize the modified influenza B HA polypeptide in the prefusion conformation through interface stabilization comprise at least one cavity filling mutation in the stem region and/or one or more amino acid mutations in the head and/or stem region to form a polar interaction with a neighboring amino acid residue; optionally wherein:
(i) the at least one cavity filling mutation is at amino acid position 460, 467, and/or 474 as indexed by reference to the amino acid sequence of SEQ ID NO: 1, e.g., a substitution at amino acid position 460 with a Lysine (K), at amino acid position 467 with a Phenylalanine (F) or a Tyrosine (Y), and/or at amino acid position 474 with a Glutamine (Q); and/or
(ii) the polar interaction comprises a salt bridge or a hydrogen bond.

Embodiment 7. The modified influenza B HA polypeptide of any one of Embodiments 1-6, wherein the one or more amino acid mutations that stabilize the modified influenza B HA polypeptide in the prefusion conformation through interface stabilization are at amino acid positions 18, 121, 188, 226, 228, 408, 435, and/or 460 as indexed by reference to the amino acid sequence of SEQ ID NO: 1.

Embodiment 8. The modified influenza B HA polypeptide of any one of Embodiments 1-7, wherein the one or more amino acid mutations that inactivate one or more pH sensors in the head and/or stem region are at amino acid position 226, 228, 237, 383, 388, 391, 401, 405, 408, 435, 460, 474 and/or 475 as indexed by reference to the amino acid sequence of SEQ ID NO: 1.

Embodiment 9. The modified influenza B HA polypeptide of any one of Embodiments 1-8, wherein the head region comprises a receptor binding site (RBS), and wherein the at least one N-linked glycosylation motif introduced in the head region is located in or adjacent to the RBS of the head region, optionally wherein the at least one N-linked glycosylation motif introduced in the head region is generated by introducing at least one substitution at amino acid position 60, 62, 141, 143, 186, 187, 214, 216, 223, and/or 224 as indexed by reference to the amino acid sequence of SEQ ID NO: 1.

Embodiment 10. The modified influenza B HA polypeptide of any one of Embodiments 1-9, wherein the at least one N-linked glycosylation motif introduced or disrupted in the stem region is generated by introducing at least one substitution at amino acid position 28, 336, and/or 349 as indexed by reference to the amino acid sequence of SEQ ID NO: 1.

Embodiment 11. The modified influenza B HA polypeptide of any one of Embodiments 1-10, wherein the at least one amino acid mutation that reduces sialic acid binding of the modified influenza B HA polypeptide is at amino acid position 157, 177, 218, and/or 257 as indexed by reference to the amino acid sequence of SEQ ID NO: 1.

Embodiment 12. A trimeric influenza B HA polypeptide complex, comprising three copies of the modified influenza B HA polypeptide of any one of Embodiments 1-11.

Embodiment 13. An artificial nucleic acid, e.g., an mRNA, encoding the modified influenza B HA polypeptide of any one of Embodiments 1-11.

Embodiment 14. A vector comprising the artificial nucleic acid of Embodiment 13.

Embodiment 15. A host cell comprising the vector of Embodiment 14.

Embodiment 16. A composition, e.g., an immunogenic composition, comprising the modified influenza B HA polypeptide of any one of Embodiments 1-11, the trimeric influenza B HA polypeptide complex of Embodiment 12, the mRNA according to Embodiment 13, or the vector of Embodiment 14.

Embodiment 17. The composition of Embodiment 16 for use in a method of immunizing a subject.

Embodiment 18. An in vitro method of preparing a trimeric influenza B HA polypeptide complex, the method comprising culturing the host cell of Embodiment 15 in a cell culture medium, and expressing the trimeric influenza B HA polypeptide complex.

### THIRD SET OF REPRESENTATIVE EMBODIMENTS OF THE PRESENT DISCLOSURE

Embodiment 1. An artificial messenger ribonucleic acid (mRNA) encoding an influenza B HA polypeptide, wherein the influenza B HA polypeptide comprises:
a) at least one proline substitution relative to a corresponding wild-type influenza B HA polypeptide, wherein the at least one proline substitution is at amino acid position 363, 366, 371, 372, 376, 380, 383, 390, 391, 393, 395, 397, 399, 421, 430, 431, 434, 436, and/or 490 as indexed by reference to the amino acid sequence of SEQ ID NO: 1;
b) at least two cysteine substitutions relative to a corresponding wild-type influenza B HA polypeptide, wherein the at least two cysteine substitutions are at amino acid positions 20 and 387, 35 and 408, 36 and 415, 37 and 411, 125 and 431, 127 and 431, 185 and 223, 186 and 224, 186 and 239, 188 and 241, 232 and 433, 233 and 434, 239 and 276, 346 and 465, 367 and 478, 378 and 397, 380 and 397, 383 and 401, 387 and 510, 394 and 507, 394 and 510, 396 and 510, 396 and 514, 401 and 475, 430 and 437, 430 and 438, and/or 430 and 439, as indexed by reference to the amino acid sequence of SEQ ID NO: 1;
c) at least one cavity filling amino acid substitution relative to a corresponding wild-type influenza B HA polypeptide, wherein the at least one cavity filling amino acid substitution is at amino acid position 460, 467, and/or 474 as indexed by reference to the amino acid sequence of SEQ ID NO: 1;
d) one or more interface stabilization amino acid substitutions relative to a corresponding wild-type influenza B HA polypeptide, wherein the one or more interface stabilization amino acid substitutions are at amino acid position 18, 121, 188, 226, 228, 408, 435, and/or 460 as indexed by reference to the amino acid sequence of SEQ ID NO: 1;
e) one or more pH sensor knock-out amino acid substitutions relative to a corresponding wild-type influenza B HA polypeptide, wherein the one or more pH sensor knock-out amino acid substitutions are at amino acid position 226, 228, 237, 239, 383, 388, 391, 401, 405, 408, 435, 460, 474 and/or 475 as indexed by reference to the amino acid sequence of SEQ ID NO: 1;
f) at least one amino acid substitution relative to a corresponding wild-type influenza B HA polypeptide, wherein the at least one amino acid substitution generates or disrupts a N-linked glycosylation motif in the influenza B HA polypeptide and is at amino acid position 28, 60, 62, 141, 143, 186, 187, 214, 216, 223, 224, 336, and/or 349 as indexed by reference to the amino acid sequence of SEQ ID NO: 1; and/or
g) at least one amino acid substitution relative to a corresponding wild-type influenza B HA polypeptide, wherein the at least one amino acid substitution is at amino acid position 157, 177, 218, and/or 257 as indexed by reference to the amino acid sequence of SEQ ID NO: 1.

Embodiment 2. The artificial mRNA of Embodiment 1, wherein the influenza B HA polypeptide comprises:
a) two proline substitutions relative to a corresponding wild-type influenza B HA polypeptide, wherein the two proline substitutions are at amino acid positions 430 and 436 as indexed by reference to the amino acid sequence of SEQ ID NO: 1; or
b) five amino acid substitutions relative to a corresponding wild-type influenza B HA polypeptide, wherein the five amino acid substitutions are at amino acid positions 383, 401, 405, 408, and 475 as indexed by reference to the amino acid sequence of SEQ ID NO: 1.

Embodiment 3. The artificial mRNA of Embodiment 2, wherein the influenza B HA polypeptide comprises amino acid substitutions:
a) A430P and N436P; or
b) H383M, S401V, A405V, K408M, and H475M,
as indexed by reference to the amino acid sequence of SEQ ID NO: 1.

Embodiment 4. The artificial mRNA of any one of Embodiments 1-3, wherein the influenza B HA polypeptide is from a B/Victoria influenza virus.

Embodiment 5. The artificial mRNA of Embodiment 5, wherein the B/Victoria influenza virus is B/Austria/1359417/2021.

Embodiment 6. The artificial mRNA of any one of Embodiments 1-5, wherein the influenza B HA polypeptide comprises an amino acid sequence having at least about 90% sequence identity to the amino acid sequence of SEQ ID NO: 3 or SEQ ID NO: 5.

Embodiment 7. The artificial mRNA of Embodiment 6, wherein the influenza B HA polypeptide comprises the amino acid sequence of SEQ ID NO: 3 or SEQ ID NO: 5.

Embodiment 8. The artificial mRNA of any one of Embodiments 1-7, comprising a nucleic acid sequence having at least about 90% sequence identity to the nucleic acid sequence of SEQ ID NO: 4 or SEQ ID NO: 6.

Embodiment 9. The artificial mRNA of Embodiment 8, comprising the nucleic acid sequence of SEQ ID NO: 4 or SEQ ID NO: 6.

Embodiment 10. The artificial mRNA of any one of Embodiments 1-9, comprising a 5′-cap structure and/or a 3′-poly(A) sequence.

Embodiment 11. The artificial mRNA of any one of Embodiments 1-10, comprising at least one chemically modified nucleotide and/or a phosphorothioate bond.

Embodiment 12. The artificial mRNA of Embodiment 11, wherein the at least one chemically modified nucleotide comprises a pseudouridine, a 2′-fluoro ribonucleotide, or a 2′-methoxy ribonucleotide, optionally wherein the pseudouridine is a N1-methylpseudouridine.

Embodiment 13. A composition comprising the artificial mRNA of any one of Embodiments 1-12 encapsulated in a lipid nanoparticle (LNP).

Embodiment 14. The composition of Embodiment 13, wherein the LNP comprises a cationic lipid.

Embodiment 15. The composition of Embodiment 14, wherein the cationic lipid comprises or is OF-02, cKK-E10, GL-HEPES-E3-E10-DS-3-E18-1, GL-HEPES-E3-E12-DS-4-E10, GL-HEPES-E3-E12-DS-3-E14, (4-hydroxybutyl)azanediyl]di(hexane-6,1-diyl) bis(2-hexyldecanoate) (ALC-0315), or IM-001.

Embodiment 16. The composition of Embodiment 14 or 15, wherein the LNP further comprises a polyethylene glycol conjugated (PEGylated) lipid, a cholesterol-based lipid, and a helper lipid.

Embodiment 17. The composition of Embodiment 16, wherein:
a) the PEGylated lipid comprises or is 1,2-dimyristoyl-rac-glycero-3-methoxypolyethylene glycol-2000 (DMG-PEG2000); and/or
b) the cholesterol-based lipid comprises or is cholesterol; and/or
c) the helper lipid comprises or is dioleoyl-sn-glycero-3-phosphoethanolamine (DOPE).

Embodiment 18. The composition of Embodiment 16 or 17, wherein:
a) the cationic lipid is present at a molar ratio between about 35% and about 55%;
b) the PEGylated lipid is present at a molar ratio between about 0.25% and about 2.75%;
c) the cholesterol-based lipid is present at a molar ratio between about 20% and about 45%; and
d) the helper lipid is present at a molar ratio between about 5% and about 35%, wherein all of the molar ratios are relative to the total lipid content of the LNP.

Embodiment 19. The composition of Embodiment 18, wherein:
a) the cationic lipid is present at a molar ratio of about 40%;
b) the PEGylated lipid is present at a molar ratio of about 1.5%;
c) the cholesterol-based lipid is present at a molar ratio of about 28.5%; and
d) the helper lipid is present at a molar ratio of about 30%,
wherein all of the molar ratios are relative to the total lipid content of the LNP.

Embodiment 20. The composition of Embodiment 19, wherein the artificial mRNA encodes the influenza B HA polypeptide of SEQ ID NO: 3, and wherein the LNP comprises GL-HEPES-E3-E12-DS-4-E10 at a molar ratio of about 40%, DMG-PEG2000 at a molar ratio of about 1.5%, cholesterol at a molar ratio of about 28.5%, and DOPE at a molar ratio of about 30%.

Embodiment 21. The composition of Embodiment 19, wherein the artificial mRNA encodes the influenza B HA polypeptide of SEQ ID NO: 5, and wherein the LNP comprises GL-HEPES-E3-E12-DS-4-E10 at a molar ratio of about 40%, DMG-PEG2000 at a molar ratio of about 1.5%, cholesterol at a molar ratio of about 28.5%, and DOPE at a molar ratio of about 30%.

Embodiment 22. The composition of any one of Embodiments 13-21, wherein the composition is an immunogenic composition.

Embodiment 23. An influenza B HA polypeptide comprising one or more amino acid substitutions relative to a corresponding wild-type influenza B HA polypeptide, wherein the one or more amino acid substitutions comprises:
a) two proline substitutions at amino acid positions 430 and 436 as indexed by reference to the amino acid sequence of SEQ ID NO: 1; or
b) amino acid substitutions at amino acid positions 383, 401, 405, 408, and 475 as indexed by reference to the amino acid sequence of SEQ ID NO: 1.

Embodiment 24. The influenza B HA polypeptide of Embodiment 23, comprising amino acid substitutions:
a) A430P and N436P; or
b) H383M, S401V, A405V, K408M, and H475M,
as indexed by reference to the amino acid sequence of SEQ ID NO: 1.

Embodiment 25. The influenza B HA polypeptide of Embodiment 23 or 24, wherein the influenza B HA polypeptide is from a B/Victoria influenza virus.

Embodiment 26. The influenza B HA polypeptide of Embodiment 25, wherein the B/Victoria influenza virus is B/Austria/1359417/2021.

Embodiment 27. The influenza B HA polypeptide of any one of Embodiments 23-26, comprising an amino acid sequence having at least about 90% sequence identity to the amino acid sequence of SEQ ID NO: 3 or SEQ ID NO: 5.

Embodiment 28. The influenza B HA polypeptide of Embodiment 27, wherein the influenza B HA polypeptide comprises the amino acid sequence of SEQ ID NO: 3 or SEQ ID NO: 5.

Embodiment 29. A trimeric influenza B HA polypeptide complex, comprising three copies of the influenza B HA polypeptide of any one of Embodiments 23-28.

Embodiment 30. An artificial nucleic acid encoding the influenza B HA polypeptide of any one of Embodiments 23-28.

Embodiment 31. The artificial nucleic acid of Embodiment 30, wherein the artificial nucleic acid comprises at least one chemically modified nucleotide and/or a phosphorothioate bond.

Embodiment 32. A vector comprising the artificial nucleic acid of Embodiment 30 or 31.

Embodiment 33. The vector of Embodiment 32, wherein the vector is a messenger RNA (mRNA) production vector.

Embodiment 34. A host cell comprising the vector of Embodiment 32 or 33.

Embodiment 35. A composition comprising the influenza B HA polypeptide of any one of Embodiments 23-28, the trimeric influenza B HA polypeptide complex of Embodiment 29, the artificial nucleic acid of Embodiment 30 or 31, or the vector of Embodiment 32 or 33.

Embodiment 36. The composition of Embodiment 35, wherein the composition is an immunogenic composition.

Embodiment 37. A vaccine comprising the composition of Embodiment 22 or 36, and a pharmaceutically acceptable carrier.

Embodiment 38. The vaccine of Embodiment 37, further comprising an adjuvant.

Embodiment 39. The vaccine of Embodiment 37 or 38, wherein the vaccine is an mRNA vaccine, and wherein the vaccine further comprises an mRNA encoding an influenza H3 HA polypeptide and an mRNA encoding an influenza H1 HA polypeptide.

Embodiment 40. The vaccine of Embodiment 37 or 38, wherein the vaccine is an mRNA vaccine, and wherein the vaccine further comprises an mRNA encoding an influenza H3 HA polypeptide, an mRNA encoding an influenza H1 HA polypeptide, an mRNA encoding an influenza N2 neuraminidase (NA) polypeptide, an mRNA encoding an influenza N1 NA polypeptide, and an mRNA encoding an influenza NA polypeptide from an influenza B/Victoria lineage.

Embodiment 41. The vaccine of Embodiment 37 or 38, wherein the vaccine is a recombinant vaccine, and wherein the vaccine further comprises an influenza H3 HA polypeptide and an influenza H1 HA polypeptide.

Embodiment 42. The vaccine of Embodiment 37 or 38, wherein the vaccine is a recombinant vaccine, and wherein the vaccine further comprises an influenza H3 HA polypeptide, an influenza H1 HA polypeptide, an influenza N2 NA polypeptide, an influenza N1 NA polypeptide, and an influenza NA polypeptide from an influenza B/Victoria lineage.

Embodiment 43. A method of immunizing a subject, the method comprising administering to the subject in need thereof the vaccine of any one of Embodiments 37-42.

Embodiment 44. The method of Embodiment 43, wherein the method prevents an influenza B virus infection in the subject, decreases the subject's likelihood of getting an influenza B virus infection, or reduces the subject's likelihood of getting serious illness from an influenza B virus infection.

Embodiment 45. The method of Embodiment 43 or 44, wherein the subject is a human.

Embodiment 46. The method of Embodiment 45, wherein the human is 6 months of age or older, less than 18 years of age, at least 6 months of age and less than 18 years of age, at least 18 years of age and less than 65 years of age, at least 6 months of age and less than 5 years of age, at least 5 years of age and less than 65 years of age, at least 60 years of age, or at least 65 years of age.

Embodiment 47. The method of any one of Embodiments 43-46, wherein the vaccine is administered intramuscularly, intradermally, subcutaneously, intravenously, intranasally, by inhalation, or intraperitoneally.

Embodiment 48. A method of reducing one or more symptoms of an influenza B virus infection, the method comprising administering to a subject in need thereof the vaccine of any one of Embodiments 37-42.

Embodiment 49. An in vitro method of preparing a trimeric influenza B HA polypeptide complex, the method comprising culturing the host cell of Embodiment 34 in a cell culture medium, and expressing the trimeric influenza B HA polypeptide complex.

Embodiment 50. The in vitro method of Embodiment 49, further comprising a step of purifying the trimeric influenza B HA polypeptide complex from the cell culture medium.

### EXAMPLES

The following examples are to be considered illustrative and not limiting on the scope of the present disclosure described above.

### Example 1. Modified Influenza B Hemagglutinin (HA) Polypeptides with Designs to Stabilize Prefusion Conformation

This example illustrates the design and preparation of various modified influenza B HA polypeptides with one or more introduced amino acid mutations that contribute to the stabilization of the modified influenza B HA polypeptides in the prefusion conformation. The structure of an influenza B HA trimer in the prefusion conformation is shown in FIG. 1. The working hypothesis was that a HA trimer in its prefusion, closed conformation would elicit better neutralizing responses in animals than a HA trimer presented in a more open conformation, such as postfusion conformation. Given that influenza HA transitions to postfusion conformation upon pH drop, an attempt was made to introduce pH sensor inactivating mutations at the HA2 stem and at the HA1 head interface.

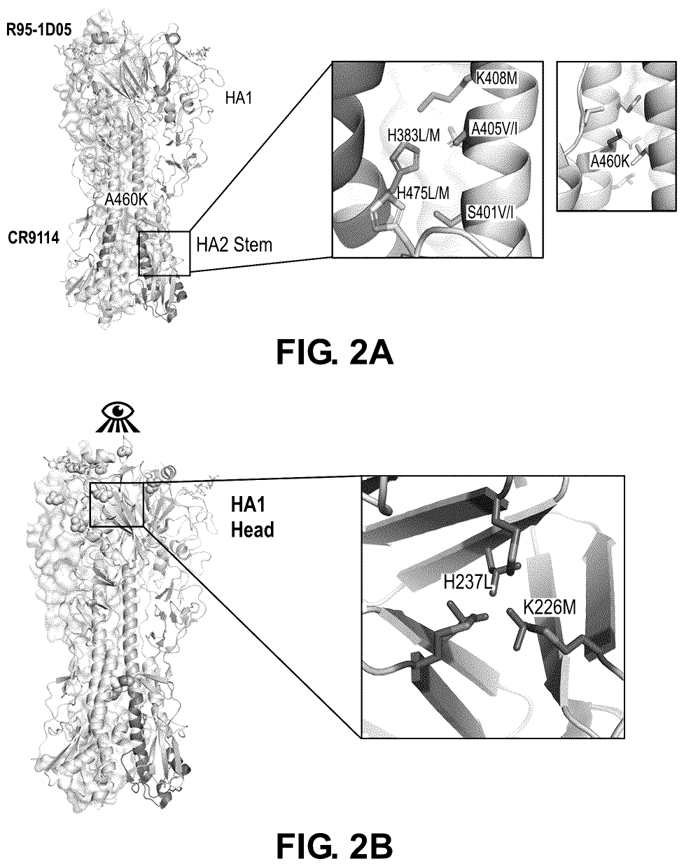

In the HA2 stem, amino acid substitutions were introduced in the polypeptides at positions 383, 401, 405, 408, and/or 475 (FIG. 2A). In an attempt to increase stem interface stabilization, an amino acid substitution was introduced in the polypeptides at position 460 (FIG. 2A). To try and increase head interface stabilization, substitutions were introduced in the polypeptides at positions 226 and/or 237 (FIG. 2B). It is noted that the numbering of the amino acid positions shown in FIG. 2A and FIG. 2B is as indexed by reference to the amino acid of the wild-type influenza B HA from B/PHUKET/3073/2013 (a B/Yamagata lineage; SEQ ID NO: 1).

In an attempt to knockout pH sensor at the HA2 stem, stabilize stem interface, and/or stabilize head interface, amino acid substitutions were introduced into the wild-type influenza B HA from B/PHUKET/3073/2013 (a B/Yamagata lineage; SEQ ID NO: 1) and the wild-type influenza B HA from B/Austria/1359417/2021 (a B/Victoria lineage; SEQ ID NO: 2) as summarized in Table 4. Monoclonal antibodies to well-defined, structural epitopes were selected against the surface of influenza B HA to ensure that the protein was properly folded and presented at the cell surface. A stem antibody (CR9114), a vestigial esterase (VE) antibody (CR8071), and a receptor binding site (RBS) antibody (R95-1D05) were used to investigate the conformation of modified influenza B HA on the surface of cells using flow cytometry. The binding of R95-1D05 indicates the total surface expression and integrity of the sialic acid binding pocket on surface expressed HAs. The binding of CR8071 and CR9114 indicates that the surface expressed HA is in a prefusion conformation and is more likely to elicit a more potent neutralizing antibody response when used in vivo as an immunogen. The binding ratio of CR9114 to R95-1D05 (CR9114 MFI/R95-1D05 MFI) suggests the ratio of prefusion, closed HA to total HA presented on the cell surface, with a higher ratio suggesting that a higher percentage of the surface expressed HA is in a prefusion, locked conformation.

TABLE 4
Structural designs and rationale of modified influenza B HA polypeptides with the aim
to knockout pH sensor at the HA2 stem, stabilize stem interface, and/or stabilize head interface.

| Construct | | | |
|---|---|---|---|
| Aus^{a} | Phu^{b} | Design^{c} | Rationale |
| 1_Aus | 17_Phu | Base Sequence | Base sequence, codon |
| | | | optimized; baseline control |
| 2_Aus | 18_Phu | A460K | Stem interface stabilization; |
| | | | variant of positive control 1 |
| 5_Aus | 21_Phu | H475L_H383L | Inactivate lower stem pH |
| | | | sensor by replacing/creating |
| | | | new hydrophobic contacts |
| 6_Aus | 22_Phu | H475L_H383L_A460K | Inactivate lower stem pH |
| | | | sensor by replacing/creating |
| | | | new hydrophobic contacts + |
| | | | stem interface stabilization |
| 7_Aus | 23_Phu | H475L_H383L_A460K_K226M_H237L | Inactivate 2 (stem & head) pH |
| | | | sensors by replacing/creating |
| | | | new hydrophobic contacts + |
| | | | stem interface stabilization |
| 8_Aus | 24_Phu | H475L_H383L_K226M_H237L | Inactivate 2 (stem & head) pH |
| | | | sensors by replacing/creating |
| | | | new hydrophobic contacts |
| 9_Aus | 25_Phu | H475L_H383L_K226M_T228V_H237L | Inactivate 2 (stem & head) pH |
| | | | sensors by replacing/creating |
| | | | new hydrophobic contacts |
| 10_Aus | 26_Phu | H475L_H383L_S401I_A405I | Inactivate lower stem pH |
| | | | sensor by replacing/creating |
| | | | new hydrophobic contacts |
| 11_Aus | 27_Phu | H475L_H383L_S401V | Inactivate lower stem pH |
| | | | sensor by replacing/creating |
| | | | new hydrophobic contacts |
| 12_Aus | 28_Phu | H475L_H383L_S401V_A405V | Inactivate lower stem pH |
| | | | sensor by replacing/creating |
| | | | new hydrophobic contacts |
| 13_Aus | 29_Phu | H475L_H383L_S401V_K408M | Inactivate lower stem pH |
| | | | sensor by replacing/creating |
| | | | new hydrophobic contacts |
| 14_Aus | 30_Phu | H475L_H383L_S401V_K408M_A405V | Inactivate lower stem pH |
| | | | sensor by replacing/creating |
| | | | new hydrophobic contacts |
| 15_Aus | 31_Phu | H475M_H383M_S401V_K408M | Inactivate lower stem pH |
| | | | sensor by replacing/creating |
| | | | new hydrophobic contacts |
| 16_Aus | 32_Phu | H475M_H383M_S401V_K408M_A405V | Inactivate lower stem pH |
| | | | sensor by replacing/creating |
| | | | new hydrophobic contacts |

^{a}Aus = B/Austria/1359417/2021|EPI1845793 (a B/Victoria lineage).
^{b}Phu = B/PHUKET/3073/2013 (a B/Yamagata lineage).
^{c}The numbering of the amino acid positions is as indexed by reference to the amino acid sequence of SEQ ID NO: 1.

To assess the surface expression and presentation of both the wild-type baseline (1_Aus and 17-Phu) and the modified influenza B HA designs, 239FT cells were transfected with a single DNA plasmid encoding each individual HA design. One day after transfection, cells were harvested, washed, and stained for analysis by flow cytometry. All cells were first stained with live/dead dye to assess cell viability. 293FT cells expressing each construct were then divided into four staining reactions and stained with primary antibodies including CR9114, CR8071, R95-1D05, or left in staining buffer alone. All reactions were then stained with a secondary antibody against human IgG conjugated to Alexa Fluor 488. Cells were then fixed with paraformaldehyde. Cells were washed in staining buffer following each of the previously described steps. Cells were then analyzed for monoclonal antibody binding using a Cytek Aurora flow cytometer with a five-laser configuration. Data was analyzed using FlowJo 10.8.1.

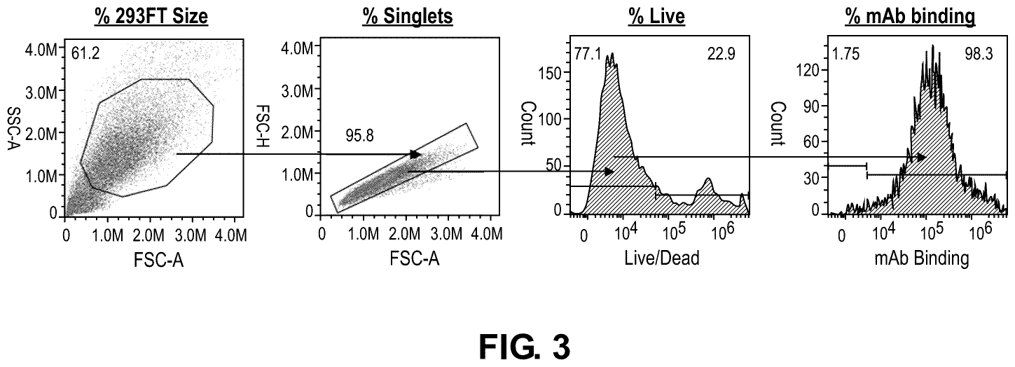

The gating scheme for flow cytometry data analyzing surface expressed HA on 293FT cells is shown in FIG. 3. Briefly, samples were processed by gating 293FT cells by size (FIG. 3, “% 293FT Size”), followed by singlets using forward-scatter height by forward-scatter area (FIG. 3, “% Singlets”), followed by live cells contain no live/dead dye (FIG. 3, “% Live”), and followed by gating on cells that bind to monoclonal antibodies of interest (FIG. 3, “% mAb binding”). Cells in the final gate “% mAb binding” were used for all downstream analysis.

As summarized in Table 4, 13 modified/Austria/1359417/2021 HA designs (constructs 2_Aus and 5_Aus through 16_Aus) were transfected as DNA plasmids into 293FT cells and assessed for their expression levels on the surface of 293FT cells using the gating scheme shown in FIG. 3 to gate single, live, 293FT cells capable of binding to monoclonal antibodies of interest against stem region, VE, and RBS and compared to a baseline HA sequence (construct 1_Aus; baseline control). All modified B/Austria/1359417/2021 HA constructs generated in this Example expressed and bound to monoclonal antibodies against the stem region, VE, and RBS with little background signal.

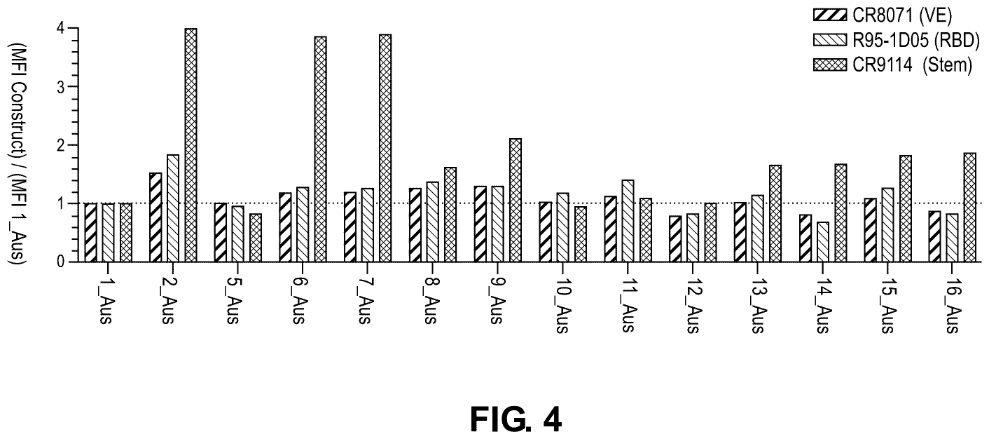

The assessment of surface expression levels of the modified B/Austria/1359417/2021 HA designs on 293FT cells measured by flow cytometry is shown in FIG. 4. The graph depicts the normalized median fluorescence intensity (MFI) of live cells that bound to each individual monoclonal antibody targeting HA. Bars with diagonal stripes depict CR8071 (VE) binding, bars with horizontal stripes depict R95-1D05 (RBS) binding, and bars with checker board depict CR9114 (stem) binding. As shown in FIG. 4, constructs 2_Aus, 6_Aus, and 7_Aus bound to the stem antibody at an increased MFI compared to both VE and RBS monoclonal antibodies as well as compared to the baseline control (construct 1_Aus), suggesting that these constructs present the stem epitope in an improved manner compared to the baseline control (construct 1_Aus) as well as the other modified influenza B HA constructs tested.

The ratio of stem to head binding monoclonal antibodies against the modified B/Austria/1359417/2021 HAs measured by flow cytometry was also assessed using the normalized ratio of stem to head binding antibodies MFI for each construct. Data were calculated as (MFI of CR9114 binding/MFI of R95-1D05 binding) per construct. These values were then normalized by the ratio of stem to head binding antibodies for construct 1_Aus (baseline control). This assessment identified constructs 2_Aus, 6_Aus, 7_Aus, 14_Aus, and 16_Aus as having an improved stem to RBS binding antibody ratio, suggesting that these modified influenza B HA polypeptides are presented in a more closed, prefusion conformation than the remaining constructs.

The same modifications were also used to generate 13 novel Phuket HA designs (constructs 18_Phu and 21_Phu through 32_Phu). These 13 modified B/PHUKET/3073/2013 HA designs were transfected as DNA plasmids into 293FT cells and assessed for their expression levels on the surface of 293FT cells using the same gating scheme shown in FIG. 3 to gate single, live, 293FT cells capable of binding to monoclonal antibodies of interest against stem, VE, and RBS and compared to the baseline control (construct 17_Phu). All modified B/PHUKET/3073/2013 HA constructs expressed and bound to monoclonal antibodies against stem, VE, and RBS with little background signal.

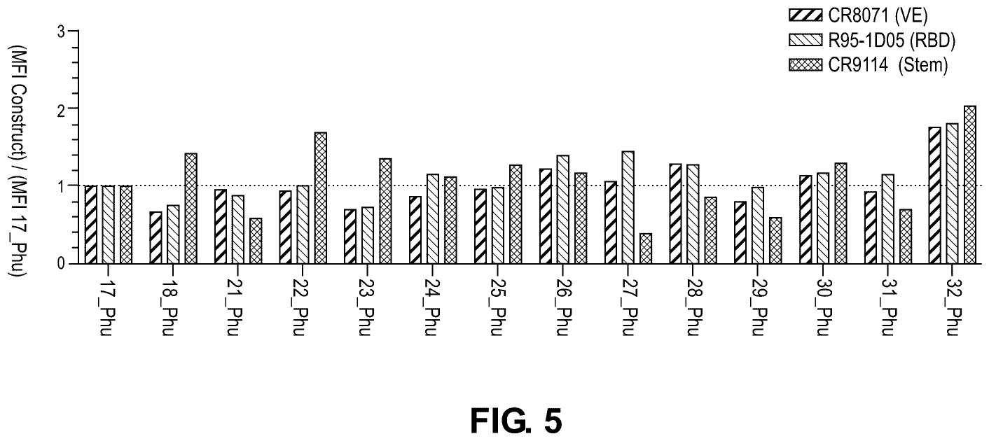

The assessment of surface expression levels of the modified B/PHUKET/3073/2013 HA designs on 293FT cells measured by flow cytometry is shown in FIG. 5. The graph depicts the normalized MFI of live cells that bound to each individual monoclonal antibody targeting HA. Bars with diagonal stripes depict CR8071 (VE) binding, bars with horizontal stripes depict R95-1D05 (RBS) binding, and bars with checker board depict CR9114 (stem) binding. As shown in FIG. 5, constructs 18_Phu, 22_Phu, and 23_Phu bound to the stem antibody at an increased MFI compared to both VE and RBS monoclonal antibodies as well as compared to the baseline control (construct 17_Phu), suggesting that these constructs present the stem epitope in an improved manner compared to the baseline control (construct 17_Phu) as well as the other modified influenza B HA constructs tested.

The ratio of stem to head binding monoclonal antibodies against the modified B/PHUKET/3073/2013 HAs measured by flow cytometry was also assessed using the normalized ratio of stem to head binding antibodies MFI for each construct. Data were calculated as (MFI of CR9114 binding/MFI of R95-1D05 binding) per construct. These values were then normalized by the ratio of stem to head binding antibodies for construct 17_Phu (baseline control). This assessment identified constructs 18_Phu, 22_Phu, and 23_Phu as having an improved stem to RBS binding antibody ratio, suggesting that these modified influenza B HA polypeptides are presented in a more closed, prefusion conformation than the remaining constructs.

### Example 2. Additional Modification Designs to Improve Stability in Prefusion Conformation, to Reduce Cell Surface Binding, and/or to Elicit Neutralizing Antibody-Focused Responses

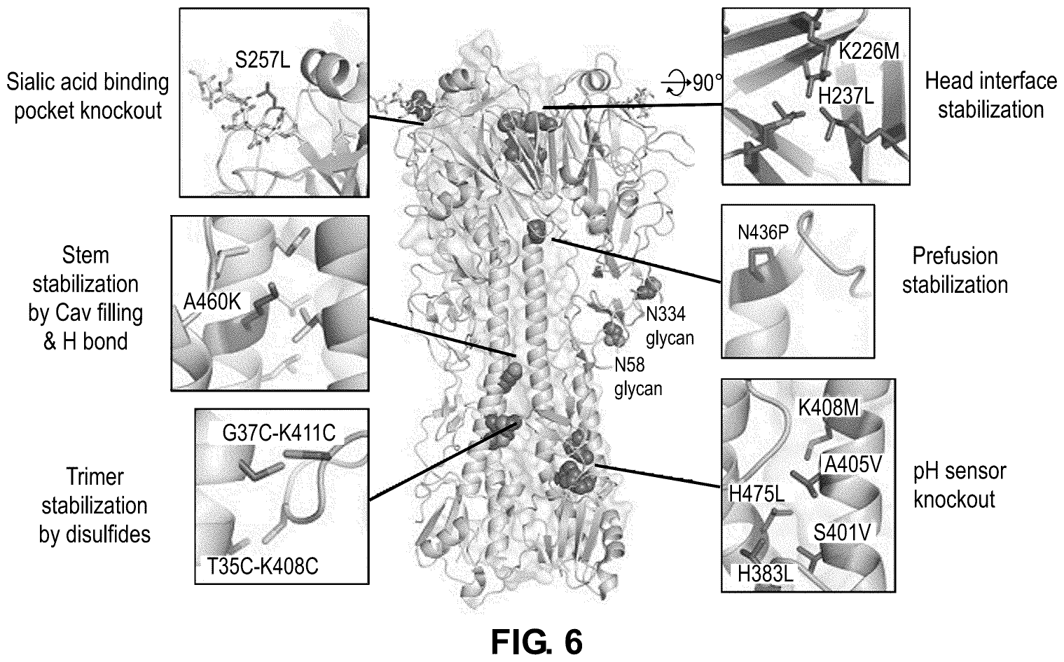

Additional modification designs were made to the modified influenza B HA polypeptides in an attempt to improve stability in prefusion conformation, to reduce cell surface binding, and/or to elicit neutralizing antibody-focused responses. The working hypothesis was that stabilizing the HA in the prefusion-closed conformation would elicit an increased neutralizing response, reducing HA binding to sialic acid would increase exposure of the RBS neutralizing epitopes to the immune system, and masking non-neutralizing epitopes would help elicit a neutralizing response. More specifically, cavity filling mutations were made in the stem region to further stabilize pH sensitive stem and head interfaces. Proline mutations were placed at the fusion peptide or the refolding region 1 (RR1). At the mid stem region, Lys substitutions were introduced to fill the cavity among central helices and to form polar interaction with a neighboring residue. Inter-protomer disulfides bonds were placed between a loop region (analogous to 30-loop from influenza A strain) of HA1 and a helix of HA2 by introducing two Cys substitutions. Amino acid substitutions were also introduced at the receptor binding site (RBS) to disrupt sialic acid binding while maintaining the integrity of the epitope targeted by RBS neutralizing antibodies. Exemplified amino acid substitutions are shown in FIG. 6. It is noted that the numbering of the amino acid positions shown in FIG. 6 is as indexed by reference to the amino acid sequence of SEQ ID NO: 1.

Similar to Example 1, amino acid substitutions were introduced into the wild-type influenza B HA from B/PHUKET/3073/2013 (a B/Yamagata lineage; SEQ ID NO: 1) and the wild-type influenza B HA from B/Austria/1359417/2021 (a B/Victoria lineage; SEQ ID NO: 2) as summarized in Table 5. The same monoclonal antibodies to well-defined, structural eptiopes, namely CR9114 (stem antibody), CR8071 (vestigial esterase antibody), and R95-1D05 (RBS antibody), were used to assess the conformation of the modified influenza B HA on the surface of cells using flow cytometry. The binding of R95-1D05 indicates the total surface expression and integrity of the sialic acid binding pocket on surface expressed HAs. The binding of CR8071 and CR9114 indicates that the surface expressed HA is in a prefusion conformation and is more likely to elicit a more potent neutralizing antibody response when used in vivo as an immunogen. The binding ratio of CR9114 to R95-1D05 (CR9114 MFI/R95-1D05 MFI) suggests the ratio of prefusion, closed HA to total HA presented on the cell surface, with a higher ratio suggesting that a higher percentage of the surface expressed HA is in a prefusion, locked conformation.

TABLE 5
Structural designs and rationale of additional modified influenza B HA polypeptides
with the aim to improve stability in prefusion conformation, to reduce cell surface binding, and/or
to elicit neutralizing antibody-focused responses.

| Construct | | | |
|---|---|---|---|
| Aus^{a} | Phu^{b} | Design^{c} | Rationale |
| 1_Aus | 17_Phu | Base Sequence | Baseline construct |
| 33_Aus | 60_Phu | A366P | Proline stabilization, |
| | | | proline helix break in |
| | | | lower stem |
| 34_Aus | 61_Phu | A380C_A397C | Disulfide bridge, SS |
| | | | bridge blocks formation of |
| | | | post-fusion helix in lower |
| | | | stem |
| 35_Aus | 62_Phu | A380P | Proline stabilization, |
| | | | proline helix break in |
| | | | lower stem |
| 36_Aus | 63_Phu | A390P | Proline stabilization, |
| | | | proline helix break in |
| | | | lower stem |
| 37_Aus | 64_Phu | A396C_A514C | Disulfide bridge, SS |
| | | | bridge blocks formation of |
| | | | post-fusion helix in lower |
| | | | stem |
| 38_Aus | 65_Phu | A397P | Proline stabilization, |
| | | | proline helix break in |
| | | | lower stem |
| 39_Aus | 66_Phu | A430P | Proline stabilization, |
| | | | proline helix break in |
| | | | upper stem |
| 40_Aus | 67_Phu | A430P_N436P | Proline stabilization, |
| | | | proline helix break in stem |
| 41_Aus | 68_Phu | E474Q | Interface stabilization, pH |
| | | | sensor interference |
| 42_Aus | 69_Phu | F363P | Proline stabilization, |
| | | | proline helix break in |
| | | | lower stem |
| 43_Aus | 70_Phu | G37C_K411C | Disulfide bridge, SS |
| | | | bridge blocks formation of |
| | | | post-fusion helix in stem |
| 45_Aus | 72_Phu | G467Q | Cavity filling, central |
| | | | stem |
| 46_Aus | 73_Phu | G467Y | Cavity filling, central |
| | | | stem |
| 47_Aus | 74_Phu | G467Y_E474Q | Disulfide bridge, SS |
| | | | bridge blocks formation of |
| | | | post-fusion helix in lower |
| | | | stem |
| 48_Aus | 75_Phu | H383C_S401C | Disulfide bridge, SS |
| | | | bridge blocks formation of |
| | | | post-fusion helix in lower |
| | | | stem |
| 49_Aus | 76_Phu | H383P | Inactivate pH sensor |
| 50_Aus | 77_Phu | H391P | Inactivate pH sensor |
| 51_Aus | 78_Phu | K60T_P336T_P28T | Glycan engineering, lower |
| | | | head, +3 glycans |
| 52_Aus | 79_Phu | K60T_P336T_P28T_D141N_E143T | Glycan engineering, head, |
| | | | +4 glycans |
| 54_Aus | 81_Phu | N232C_E433C | Disulfide bridge, links |
| | | | HA1 with HA2 preventing |
| | | | PostF HA2 conformation |
| 55_Aus | 82_Phu | N436P | Proline stabilization, |
| | | | proline helix break in |
| | | | upper stem |
| 56_Aus | 83_Phu | S401C_H475C | Disulfide bridge, SS |
| | | | bridge blocks formation of |
| | | | post-fusion helix in lower |
| | | | stem |
| 57_Aus | 84_Phu | T35C_K408C | Disulfide bridge, links |
| | | | HA1 with HA2 preventing |
| | | | PostF HA2 conformation |
| 58_Aus | 85_Phu | T349V | Glycan engineering, stem |
| | | | glycan removal |
| 59_Aus | 86_Phu | V421P | Proline stabilization, |
| | | | proline helix break in stem |

^{a}Aus = B/Austria/1359417/2021|EPI1845793 (a B/Victoria lineage).
^{b}Phu = B/PHUKET/3073/2013 (a B/Yamagata lineage).
^{c}The numbering of the amino acid positions is as indexed by reference to the amino acid sequence of SEQ ID NO: 1.

To assess the surface expression and presentation of both the wild-type baseline (1_Aus and 17_Phu) and modified influenza B HA designs, 239FT cells were transfected with a single DNA plasmid encoding each individual HA design. One day after transfection, cells were harvested, washed, and stained for analysis by flow cytometry. All cells were first stained with live/dead dye to assess cell viability. 293FT cells expressing each construct were then divided into four staining reactions and stained with primary antibodies including CR9114, CR8071, R95-1D05, or left in staining buffer alone. All reactions were then stained with a secondary antibody against human IgG conjugated to Alexa Fluor 488. Cells were then fixed with paraformaldehyde. Cells were washed in staining buffer following each of the previously described steps. Cells were then analyzed for monoclonal antibody binding using a Cytek Aurora flow cytometer with a five-laser configuration. Data was analyzed using FlowJo 10.8.1.

As summarized in Table 5, 25 modified B/Austria/1359417/2021 HA designs (constructs 33_Aus through 43_Aus, 45_Aus through 52_Aus, and 54_Aus through 59_Aus) were transfected as DNA plasmids into 293FT cells and assessed for their expression levels on the surface of 293FT cells using the same gating scheme described in Example 1 and shown in FIG. 3 to gate single, live, 293FT cells capable of binding to monoclonal antibodies of interest against stem region, VE, and RBS and compared to the baseline HA sequence (construct 1_Aus; baseline control). The majority of the modified B/Austria/1359417/2021 HA constructs generated in this Example expressed and bound to monoclonal antibodies against the stem region, VE, and RBS.

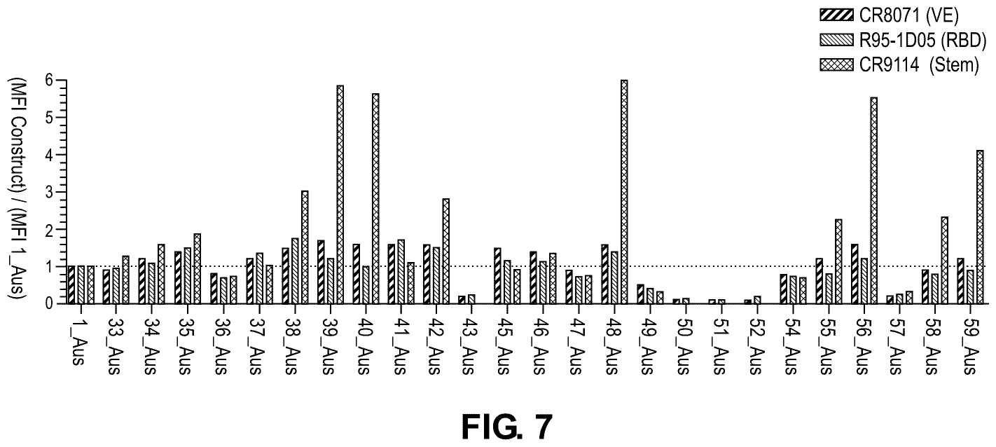

The assessment of surface expression levels of the modified B/Austria/1359417/2021 HA designs on 293FT cells measured by flow cytometry is shown in FIG. 7. The graph depicts the normalized median fluorescence intensity (MFI) of live cells that bound to each individual monoclonal antibody targeting HA. Bars with diagonal stripes depict CR8071 (VE) binding, bars with horizontal stripes depict R95-1D05 (RBS) binding, and bars with checker board depict CR9114 (stem) binding. The MFI data is normalized to the baseline control construct 1_Aus. As shown in FIG. 7, constructs 39_Aus, 40_Aus, 48_Aus, 56_Aus, and 59_Aus bound to the stem antibody at an increased MFI compared to both VE and RBS monoclonal antibodies as well as compared to the baseline controls (constructs 1_Aus), suggesting that these constructs present the stem epitope in an improved manner compared to the baseline controls (constructs 1_Aus), as well as the other novel HA constructs tested.

The ratio of stem to head binding monoclonal antibodies against modified B/Austria/1359417/2021 HAs measured by flow cytometry was also assessed using the normalized ratio of stem to head binding antibodies MFI for each construct. Data were calculated as (MFI of CR9114 binding/MFI of R95-1D05 binding) per construct. These values were then normalized by the ratio of stem to head binding antibodies for construct 1_Aus (baseline control). This assessment identified constructs 39_Aus, 40_Aus, 48_Aus, 56_Aus, and 59_Aus as having an improved stem to RBS binding antibody ratio, suggesting that these modified influenza B HA polypeptides are presented in a more closed, prefusion conformation than the remaining constructs.

The same modifications were also used to generate 25 modified B/PHUKET/3073/2013 HA designs (constructs 60_Phu through 70_Phu, 72_Phu through 79_Phu, and 81_Phu through 86_Phu). These 25 modified B/PHUKET/3073/2013 HA designs were transfected as DNA plasmids into 293FT cells and assessed for their expression levels on the surface of 293FT cells using the same gating scheme shown in FIG. 3 to gate single, live, 293FT cells capable of binding to monoclonal antibodies of interest against stem, VE, and RBS and compared to the baseline control (construct 17_Phu). The majority of the modified B/PHUKET/3073/2013 HA constructs generated in this Example expressed and bound to monoclonal antibodies against the stem region, VE, and RBS.

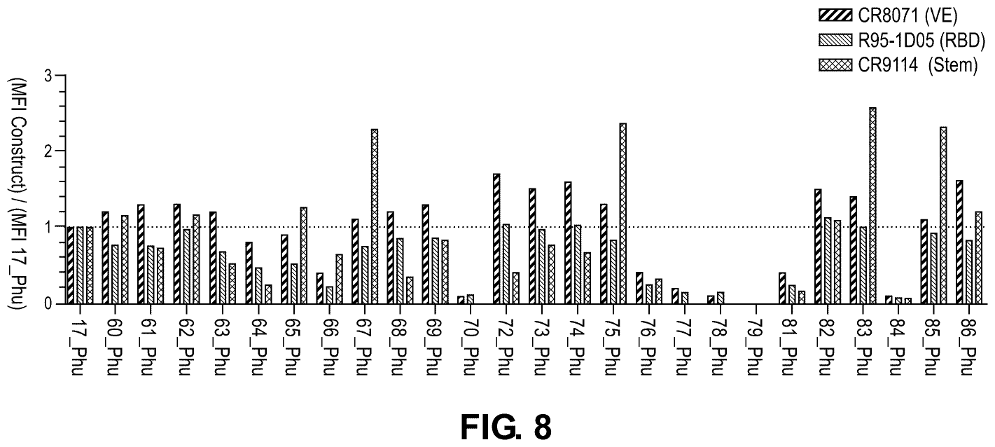

The assessment of surface expression levels of the modified B/PHUKET/3073/2013 HA designs on 293FT cells measured by flow cytometry is shown in FIG. 8. The graph depicts the normalized MFI of live cells that bound to each individual monoclonal antibody targeting HA. Bars with diagonal stripes depict CR8071 (VE) binding, bars with horizontal stripes depict R95-1D05 (RBS) binding, and bars with checker board depict CR9114 (stem) binding. As shown in FIG. 8, constructs 65_Phu, 67_Phu, 75_Phu, 83_Phu, and 85_Phu bound to the stem antibody at an increased MFI compared to both VE and RBS monoclonal antibodies as well as compared to the baseline control (construct 17_Phu), suggesting that these constructs present the stem epitope in an improved manner compared to the baseline control (construct 17_Phu), as well as the other novel HA constructs tested.

The ratio of stem to head binding monoclonal antibodies against modified B/PHUKET/3073/2013 HAs by flow cytometry was also assessed using the normalized ratio of stem to head binding antibodies MFI for each construct. Data were calculated as (MFI of CR9114 binding/MFI of R95-1D05 binding) per construct. These values were then normalized by the ratio of stem to head binding antibodies for construct 17_Phu (baseline control). This assessment identified constructs 65_Phu, 66_Phu, 67_Phu, 75_Phu, 83_Phu, and 85_Phu as having an improved stem to RBS binding antibody ratio, suggesting that these modified influenza B HA polypeptides are presented in a more closed, prefusion conformation than the remaining constructs.

### Example 3. Assessment of Additional Modified Influenza B HA Polypeptides

In this example, additional modified influenza B HA polypeptides were generated to include one or more modification designs described in Examples 1 and 2, namely proline mutations, disulfide bridge forming mutations, interface stabilizing mutations, pH sensor inactivating mutations, glycan engineering mutations, and sialic acid binding interfering mutations. Various constructs were generated by introducing amino acid substitutions into the wild-type influenza B HA from B/PHUKET/3073/2013 (a B/Yamagata lineage; SEQ ID NO: 1) and the wild-type influenza B HA from B/Austria/1359417/2021 (a B/Victoria lineage; SEQ ID NO: 2). Approximately 125 additional modified influenza B HA polypeptides were generated based on each wild-type strain.

Monoclonal antibodies to well-defined, structural epitopes were selected against the surface of influenza B HA to ensure that the protein was properly folded and presented at the cell surface. A stem-specific antibody (CR9114) and an RBS-specific antibody (R95-1D05) were used to investigate the conformation of modified influenza B HA on the surface of cells using flow cytometry. The binding of R95-1D05 indicates the total surface expression and integrity of the sialic acid binding pocket on surface expressed HAs. The binding of CR9114 indicates that the surface expressed HA is in a prefusion conformation and is more likely to elicit a more potent neutralizing antibody response when used in vivo as an immunogen. The binding ratio of CR9114 to R95-1D05 (CR9114 MFI/R95-1D05 MFI) suggests the ratio of prefusion, closed HA to total HA presented on the cell surface, with a higher ratio suggesting that a higher percentage of the surface expressed HA is in a prefusion, locked conformation.

Both the wild-type baseline (1_Aus and 17_Phu) and the newly-generated modified influenza B HA designs were transfected as DNA plasmids into 293FT cells and assessed for their expression levels on the surface of 293FT cells using the same gating scheme described in Example 1 and shown in FIG. 3 to gate single, live, 293FT cells capable of binding to monoclonal antibodies of interest against stem region and RBS and compared to the baseline HA control (constructs 1_Aus and 17_Phu). The majority of the additional B/PHUKET/3073/2013 and B/Austria/1359417/2021 HA constructs generated in this Example expressed and bound to monoclonal antibodies against the stem region and RBS.

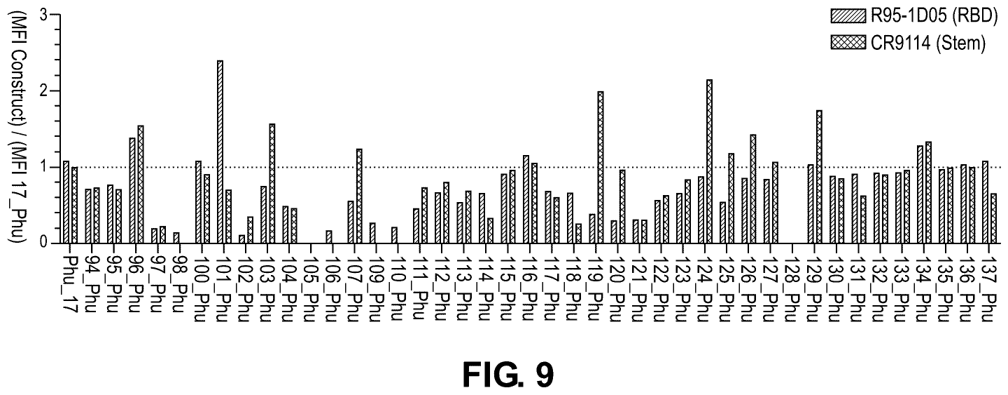

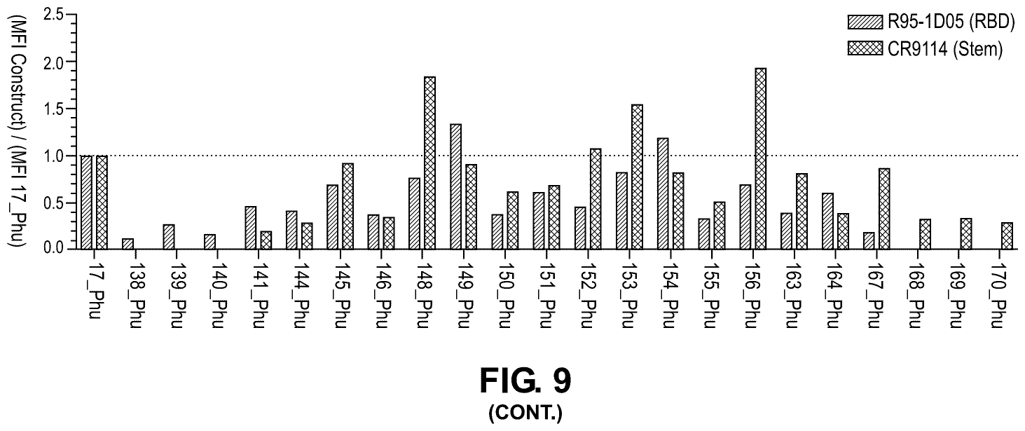

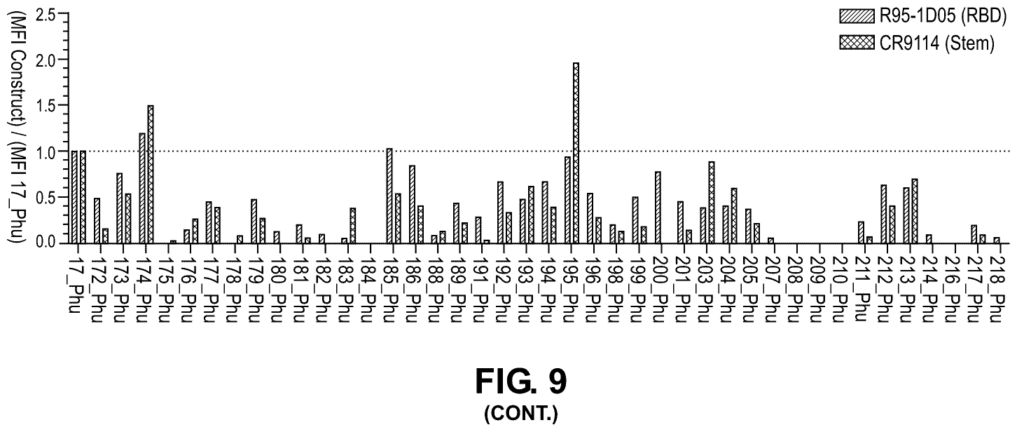

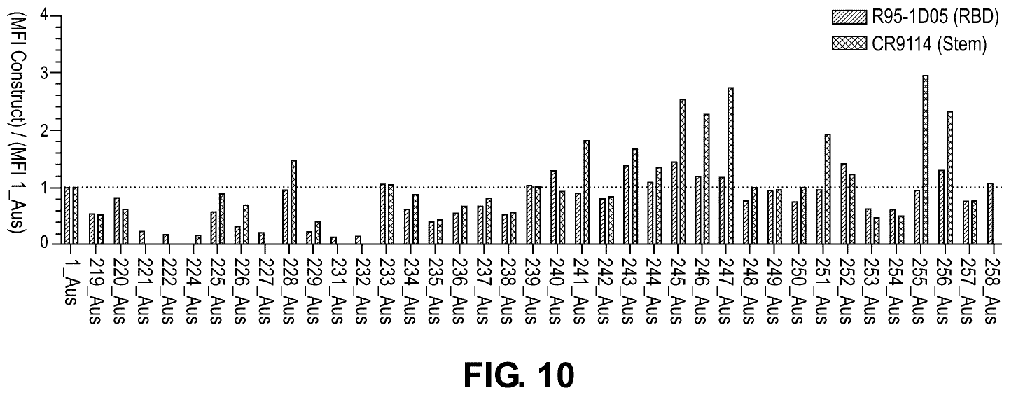

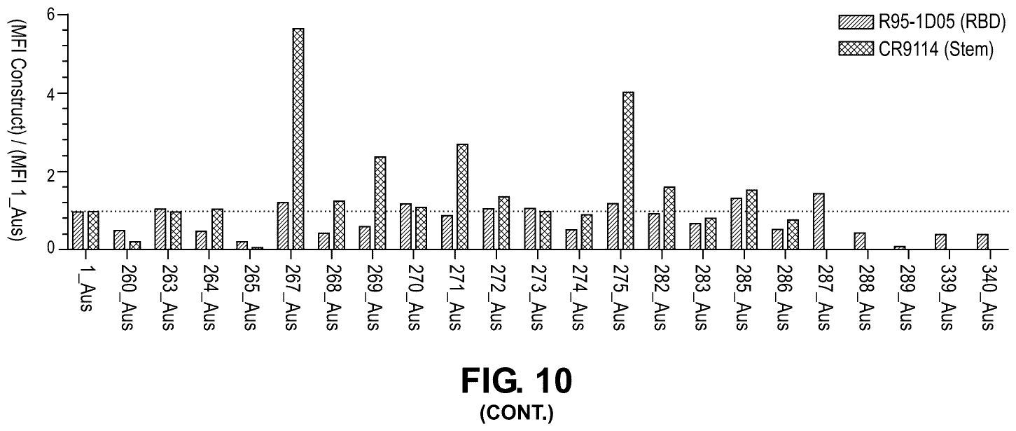

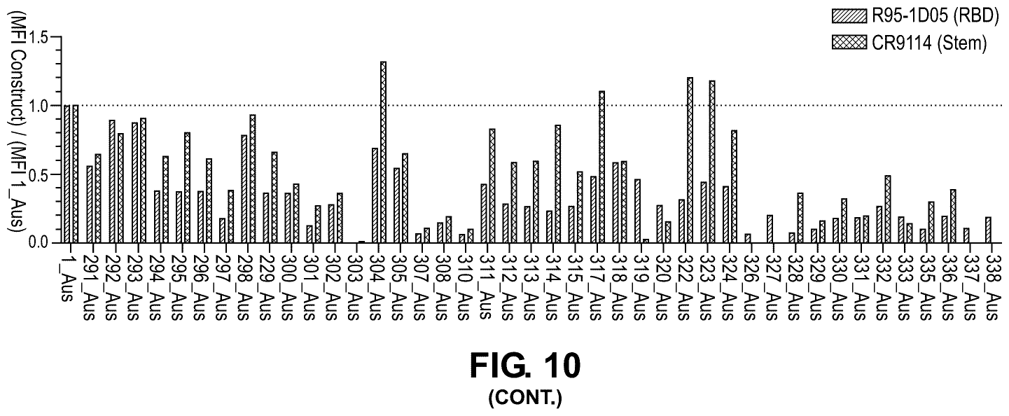

The assessment of surface expression levels of the modified B/PHUKET/3073/2013 HA constructs and the modified B/Austria/1359417/2021 HA constructs on 293FT cells measured by flow cytometry is shown in FIG. 9 and FIG. 10, respectively. The graph depicts the normalized MFI of live cells that bound to each individual monoclonal antibody targeting HA. Bars with horizontal stripes depict R95-1D05 (RBS) binding and bars with checker board depict CR9114 (stem) binding. The MFI data is normalized to the baseline control construct 1_Aus for the modified B/Austria/1359417/2021 HA constructs and the baseline control construct 17_Phu for the modified B/PHUKET/3073/2013 HA constructs. As shown in FIG. 9 and FIG. 10, while not all of the tested constructs helped to stabilize HA in the prefusion conformation, some constructs did have more stem antibody binding than RBD antibody binding, indicating that those constructs expressed more stabilized HA in the prefusion conformation.

The ratio of stem to head binding monoclonal antibodies against modified B/PHUKET/3073/2013 HAs and modified B/Austria/1359417/2021 HAs measured by flow cytometry was also assessed using the normalized ratio of stem to head binding antibodies MFI for each construct. Data were calculated as (MFI of CR9114 binding/MFI of R95-1D05 binding) per construct. These values were then normalized by the ratio of stem to head binding antibodies for construct 1_Aus or 17_Phu (baseline control). This assessment identified certain modified B/PHUKET/3073/2013 HAs and certain modified B/Austria/1359417/2021 HAs as having an improved stem to RBS binding antibody ratio (at least 2-fold increase), suggesting that those modified influenza B HA polypeptides are presented in a more closed, prefusion conformation than the remaining constructs.

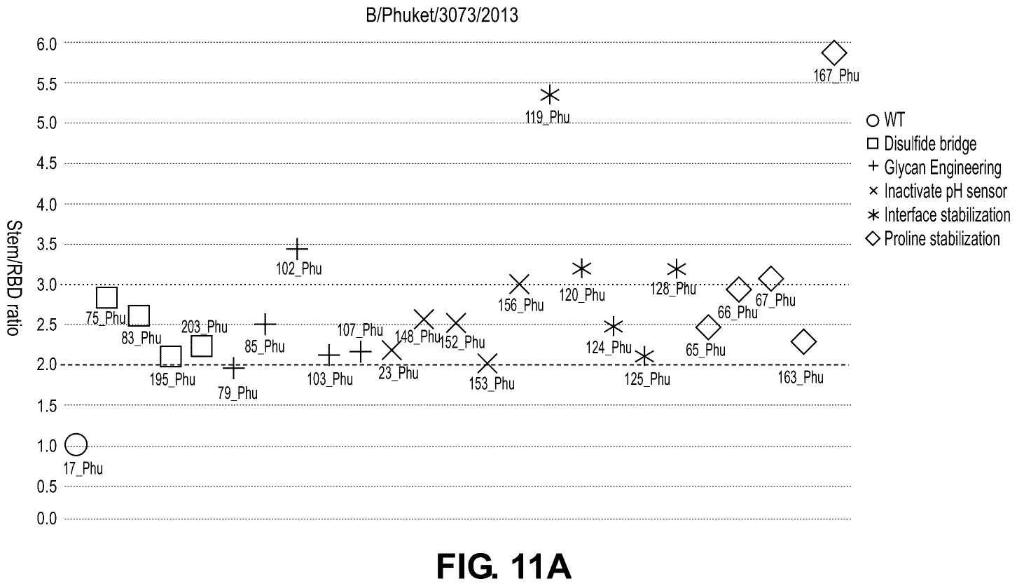

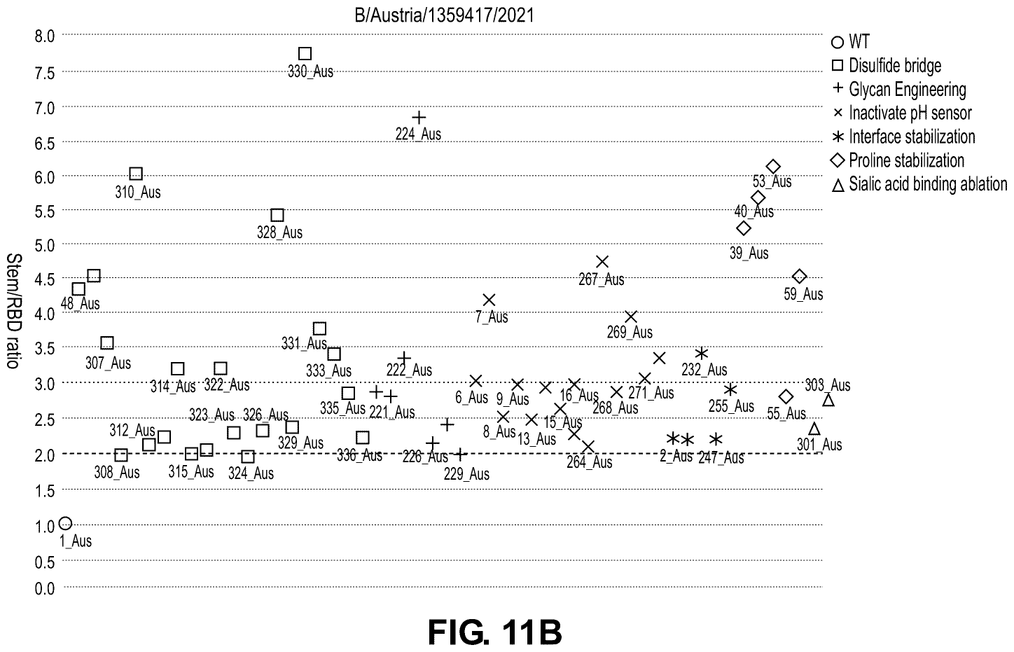

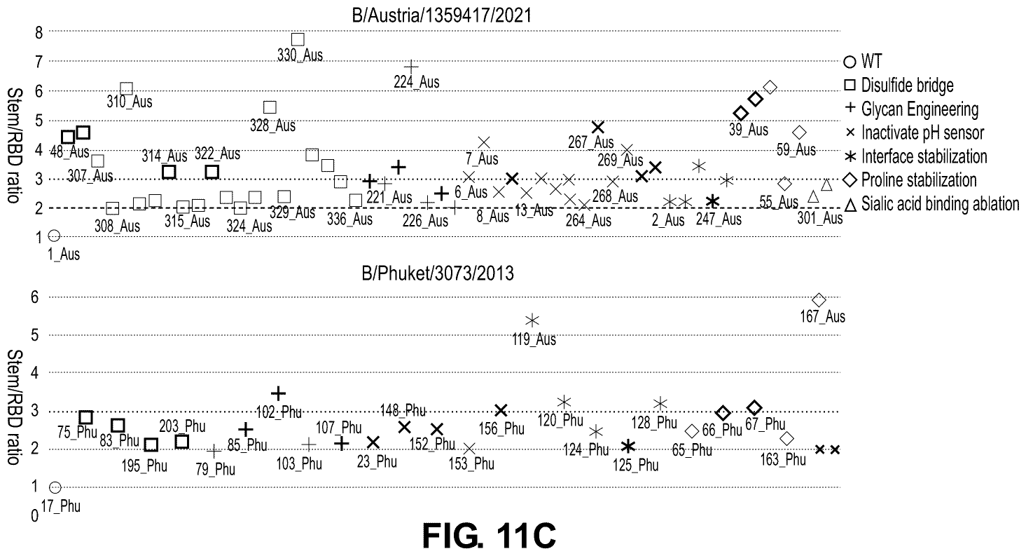

In total, 24 modified B/PHUKET/3073/2013 HAs were identified in this Example and Examples 1 and 2 as having at least 2-fold increase in stem to RBS binding antibody ratio. The structure design of these 24 modified B/PHUKET/3073/2013 HAs are provided in Table 6. Amongst these modified HAs, 5 contain proline mutations, 4 contain disulfide bridge forming mutations, 5 contain interface stabilizing mutations, 5 contain pH sensor inactivating mutations, and 5 contain glycan engineering mutations (FIG. 11A and FIG. 11D). Although none of these modified B/PHUKET/3073/2013 HAs contain sialic acid binding interfering mutations, it is possible that this is because of the stringent cut-off line (i.e., 2-fold increase in stem to RBS binding antibody ratio) selected.

TABLE 6
Structure design of the 24 modified B/PHUKET/3073/2013
HAs having at least 2-fold
increase in stem to RBS binding antibody ratio
(the numbering of the amino acid positions is asindexed
by reference to the amino acid sequence of SEQ ID NO: 1).

| Construct | Design | Type of Mutation |
|---|---|---|
| 203_Phu | L125C_M431C | Disulfide bridge |
| 195_Phu | A430C_L439C | Disulfide bridge |
| 83_Phu | S401C_H475C | Disulfide bridge |
| 75_Phu | H383C_S401C | Disulfide bridge |
| 107_Phu | T349A | Glycan Engineering |
| 103_Phu | P28T | Glycan Engineering |
| 102_Phu | Q214N_K216T | Glycan Engineering |
| 85_Phu | T349V | Glycan Engineering |
| 79_Phu | K60T_P28T_D141N_E143T_P336T | Glycan Engineering |
| 156_Phu | K408M | Inactivate pH sensor |
| 153_Phu | H475Y | Inactivate pH sensor |
| 152_Phu | H475W | Inactivate pH sensor |
| 148_Phu | H435E | Inactivate pH sensor |
| 23_Phu | K226M_H237L_H383L_A460K_H475L | Inactivate pH sensor |
| 128_Phu | Q121K | Interface stabilization |
| 125_Phu | K408R | Interface stabilization |
| 124_Phu | K226M_T228V_H237L | Interface stabilization |
| 120_Phu | K226M | Interface stabilization |
| 119_Phu | H435L | Interface stabilization |
| 167_Phu | L399P | Proline stabilization |
| 163_Phu | E372P | Proline stabilization |
| 67_Phu | A430P_N436P | Proline stabilization |
| 66_Phu | A430P | Proline stabilization |
| 65_Phu | A397P | Proline stabilization |

On the other hand, 55 modified B/Austria/1359417/2021 HAs were identified in this Example and Examples 1 and 2 as having at least 2-fold increase in stem to RBS binding antibody ratio. The structure design of these 55 modified B/Austria/1359417/2021 HAs are provided in Table 7. Amongst these modified HAs, 5 contain proline mutations, 21 contain disulfide bridge forming mutations, 5 contain interface stabilizing mutations, 15 contain pH sensor inactivating mutations, 7 contain glycan engineering mutations, and 2 contain sialic acid binding interfering mutations (FIG. 11B and FIG. 11D). The common modification designs that conferred at least 2-fold increase in stem to RBS binding antibody ratio in both B/PHUKET/3073/2013 and B/Austria/1359417/2021 strains are shown in FIG. 11C.

TABLE 7
Structure design of the 24 modified B/Austria/1359417/2021
HAs having at least 2-fold increase in
stem to RBS binding antibody ratio
(the numbering of the amino acid positions is as indexed
by reference to the amino acid sequence of SEQ ID NO: 2).

| Construct | Design | Rationale |
|---|---|---|
| 336_Aus | V237C_T274C | Disulfide bridge |
| 335_Aus | T186C_Q239C | Disulfide bridge |
| 333_Aus | S413C_A34C | Disulfide bridge |
| 56_Aus | S399C_H473C | Disulfide bridge |
| 331_Aus | S385C_L508C | Disulfide bridge |
| 330_Aus | S385C_I18C | Disulfide bridge |
| 329_Aus | P184C_V237C | Disulfide bridge |
| 328_Aus | P184C_P222C | Disulfide bridge |
| 326_Aus | N463C_A344C | Disulfide bridge |
| 324_Aus | N183C_K221C | Disulfide bridge |
| 323_Aus | M376C_A395C | Disulfide bridge |
| 322_Aus | L125C_M429C | Disulfide bridge |
| 48_Aus | H381C_S399C | Disulfide bridge |
| 317_Aus | G231C_L432C | Disulfide bridge |
| 315_Aus | A476C_I365C | Disulfide bridge |
| 314_Aus | A428C_L437C | Disulfide bridge |
| 313_Aus | A428C_I436C | Disulfide bridge |
| 312_Aus | A428C_E435C | Disulfide bridge |
| 310_Aus | A394C_L508C | Disulfide bridge |
| 308_Aus | A392C_Q505C | Disulfide bridge |
| 307_Aus | A392C_L508C | Disulfide bridge |
| 58_Aus | T347V | Glycan Engineering |
| 229_Aus | T347A | Glycan Engineering |
| 227_Aus | P334T | Glycan Engineering |
| 226_Aus | P184F_L185T | Glycan Engineering |
| 224_Aus | K221N_P222F_Q223T | Glycan Engineering |
| 222_Aus | K60T_P334T | Glycan Engineering |
| 221_Aus | K60T | Glycan Engineering |
| 275_Aus | K406M | Inactivate pH sensor |
| 9_Aus | K224M_T226V_H235L_ | Inactivate pH sensor |
| | H381L_H473L | |
| 8_Aus | K224M_H235L_H381L_ | Inactivate pH sensor |
| | H473L | |
| 7_Aus | K224M_H235L_H381L_ | Inactivate pH sensor |
| | A458K_H473L | |
| 271_Aus | H473W | Inactivate pH sensor |
| 269_Aus | H433Y | Inactivate pH sensor |
| 268_Aus | H433F | Inactivate pH sensor |
| 267_Aus | H433E | Inactivate pH sensor |
| 264_Aus | H386W | Inactivate pH sensor |
| 16_Aus | H381M_S399V_A403V_ | Inactivate pH sensor |
| | K406M_H473M | |
| 16_Aus | H381M_S399V_A403V_ | Inactivate pH sensor |
| | K406M_H473M | |
| 15_Aus | H381M_S399V_K406M_ | Inactivate pH sensor |
| | H473M | |
| 14_Aus | H381L_S399V_A403V_ | Inactivate pH sensor |
| | K406M_H473L | |
| 13_Aus | H381L_S399V_K406M_ | Inactivate pH sensor |
| | H473L | |
| 6_Aus | H381L_A458K_H473L | Inactivate pH sensor |
| 255_Aus | T186N | Interface stabilization |
| 247_Aus | K406R | Interface stabilization |
| 232_Aus | D16W | Interface stabilization |
| 3_Aus | A458R | Interface stabilization |
| 2_Aus | A458K | Interface stabilization |
| 59_Aus | V419P | Proline stabilization |
| 55_Aus | N434P | Proline stabilization |
| 53_Aus | M429P_L432P | Proline stabilization |
| 40_Aus | A428P_N434P | Proline stabilization |
| 39_Aus | A428P | Proline stabilization |
| 303_Aus | V175D | Sialic acid binding ablation |
| 301_Aus | S255L | Sialic acid binding ablation |
| 297_Aus | S155A | Sialic acid binding ablation |
| 295_Aus | L216A | Sialic acid binding ablation |

### Example 4. In Vitro Characterization of Modified Influenza B HA Polypeptides

This example describes the in vitro characterization of two representative modified influenza B HA polypeptides designed based on the HA polypeptide of B/Austria/1359417/2021 as described in the above examples. Specifically, construct 16_Aus described in Example 1 and construct 40_Aus described in Example 2 were selected for further assessment of their respective surface expression and presentation.

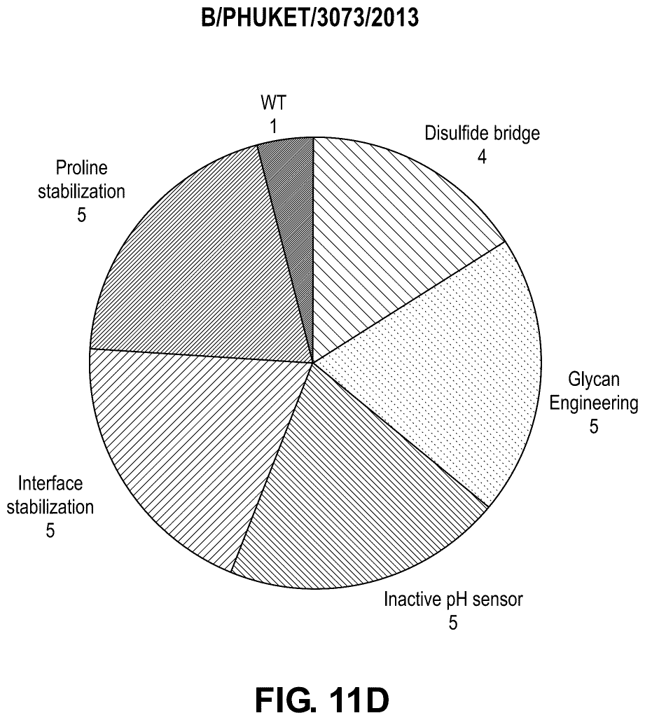

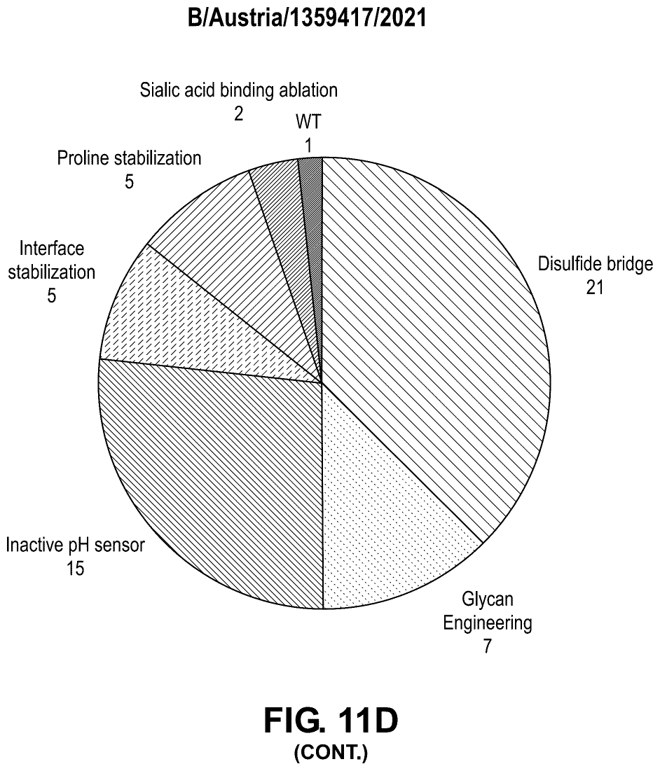

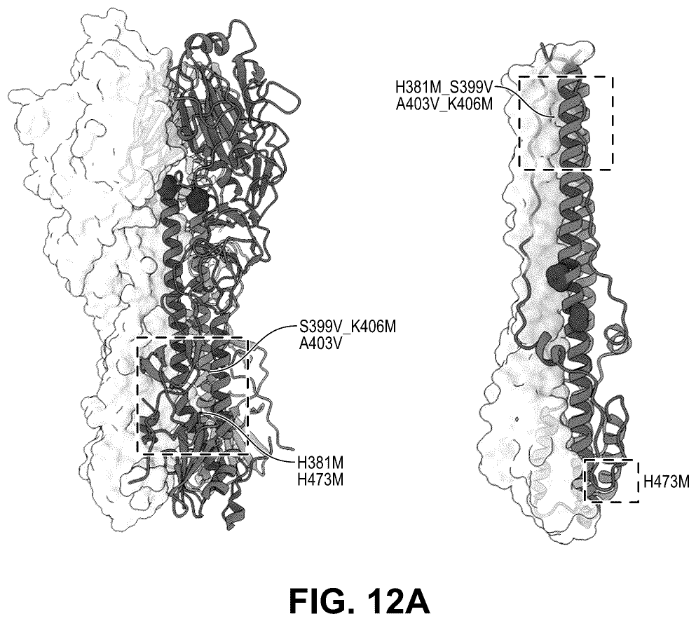

As described in Example 1 and shown in Table 4, Construct 16_Aus contains the structural design H475M_H383M_S401V_K408M_A405V, wherein the numbering of the amino acid positions is as indexed by reference to the amino acid sequence of SEQ ID NO: 1. Because of the sequence difference between SEQ ID NO: 1 and SEQ ID NO: 2, this structural design corresponds to amino acid substitutions H473M, H381M, S399V, K406M, and A403V in the wild-type influenza B HA polypeptide from B/Austria/1359417/2021 (i.e., SEQ ID NO: 2). The modeled structure of the HA trimer, using the sequence of B/Austria/1359417/2021, shows the location of the introduced amino acid substitutions (FIG. 12A). Without wishing to be bound by any theory, the amino acid substitutions H381M_H473M are predicted to reduce the sensing of the low pH environment and thus prevent the HA trimer from transitioning into a postfusion state, while hydrophobic residues (S399V_K406M_A403V) at the lower stem of the helix are predicted to fill the hydrophobic cavity and thus favors the prefusion state. This modified influenza B HA polypeptide is also referred to as the “16_Aus design.”

As described in Example 2 and shown in Table 5, Construct 40_Aus contains the structural design A430P_N436P, wherein the numbering of the amino acid positions is as indexed by reference to the amino acid sequence of SEQ ID NO: 1. Because of the sequence difference between SEQ ID NO: 1 and SEQ ID NO: 2, this structural design corresponds to amino acid substitutions A428P and N434P in the wild-type influenza B HA polypeptide from B/Austria/1359417/2021 (i.e., SEQ ID NO: 2). The modeled structure of the HA trimer, using the sequence of B/Austria/1359417/2021, shows the location of the introduced amino acid substitutions (FIG. 12B). Without wishing to be bound by any theory, after low pH triggering, the introduced prolines are in the middle of a long alpha helix (FIG. 12B, right), which strongly disfavored the postfusion conformation (only shown HA2 subunit). It is hypothesized that this design would be preferentially in the prefusion conformation. This modified influenza B HA polypeptide is also referred to as the “40_Aus design.”

Monoclonal antibodies to structural epitopes of the influenza HA protein were selected against the surface of B/Austria/1359417/2021 HA polypeptide to ensure that the protein was properly folded and presented at the cell surface and retained antigenic likeness to the wild-type protein. A stem-region-specific antibody (CR9114), a receptor binding domain (RBD)-specific antibody (R95-1D05), and an antibody (CR8071) that binds to the conserved vestigial esterase region of the HA head were used to investigate the conformation of the designs on the surface of cells using flow cytometry. The binding of R95-1D05 indicates the total surface expression and integrity of sialic acid binding pocket on surface expressed HA polypeptides. The binding of CR9114 indicates that the surface expressed HA polypeptides successfully presented a properly folded stem region and are in a prefusion state. The binding of CR8071 indicates that the vestigial esterase domain of the HA trimer head is properly folded and presented.

To assess the surface expression and presentation of the wild-type and the 40_Aus and 16_Aus designs, 239FT cells were transfected in triplicate with a single DNA plasmid (pDNA) encoding each individual HA polypeptide. 24 hours after transfection, cells were harvested, washed, and stained for analysis by flow cytometry. All cells were first stained with live/dead dye to assess cell viability. 293FT cells expressing each construct were then divided into four staining reactions and stained with primary antibodies including CR9114, R95-1D05 and CR8071 or left in staining buffer alone (Fc control not shown). All reactions were then stained with a secondary antibody against human IgG conjugated to Alexa Fluor 488. Cells were washed in staining buffer following each of the previously described steps. Cells were then analyzed for monoclonal antibody binding using a Cytek Aurora flow cytometer with a five-laser configuration. Data was analyzed using FLOWJO® 10.8.1 (FlowJo LLC).

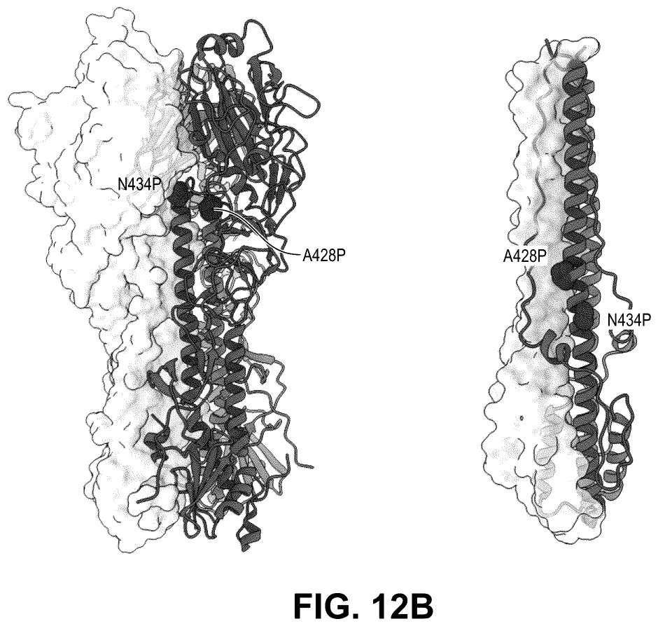

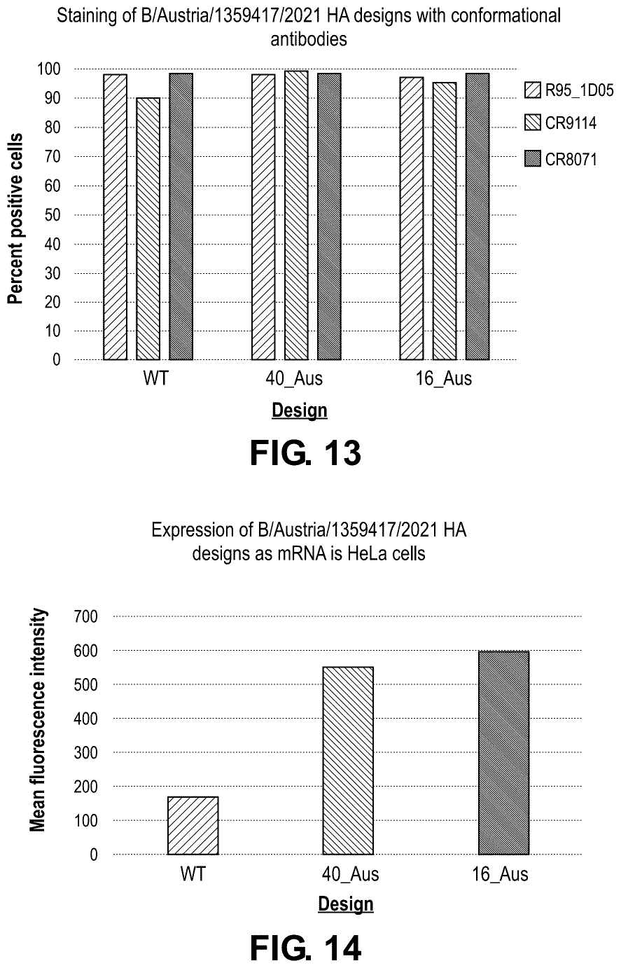

The mean fluorescent intensity (MFI) was normalized to the percent of positive stained cells for each monoclonal antibody binding measured by flow cytometry is shown in FIG. 13. The graphs depict the percentage of live cells that bound to each individual monoclonal antibody targeting HA from 30,000 cells (R95_1D05: RBD binding, CR9114: stem binding, and CR8071: VE binding). Construct names are listed on the x-axis and percent values of positive cells bound by each antibody on the y-axis. As shown in FIG. 13, the wild type (WT) and the Aus_16 design have similar binding to each of R95_1D05, CR9114 and CR8071, while the Aus_40 design has slightly higher CR9114 binding, indicating that the designs are conformationally as predicted in the prefusion state. For the 16_Aus design, the stem/RBD ratio is about 3, while for the 40_Aus design is about 6, suggesting that the designs have a higher proportion of the HA polypeptide in the prefusion conformation

### Example 5. In Vitro Expression of Influenza B HA Polypeptides

This example describes the in vitro expression of two representative influenza B HA polypeptides in mRNA-transfected HeLa cells. In this example, mRNAs encoding for the wild-type HA polypeptide from B/Austria/1359417/2021 or modified influenza B HA polypeptides from the same B/Austria/1359417/2021 strain (i.e., the 40_Aus design and the 16_Aus design) were transfected into HeLa cells that were seeded the day prior to transfection and incubated overnight at 37° C./5% CO₂ in a humidified incubator. The following day, mRNAs were transfected using Lipofectamine (following manufacturer's protocol) and cells were incubated for 20+0.5 hours at 37° C./5% CO₂ in a humidified incubator. At the experimental endpoint, cells were fixed at room temperature with 4% paraformaldehyde, washed with PBS, permeabilized with digitonin, and blocked with 10% goat serum for 1 hour at room temperature. Cells were subsequently incubated overnight with an antibody specific for influenza B HA at 4° C. overnight. The following day, cells were washed and incubated with an Alexa-647 conjugated secondary antibody, CELLMASK™ Blue, and NUCBLUE™ for 1 hour at room temperature. Image acquisition was performed on an Operetta CLS high content imaging microscope using a 20× Water objective. The background-subtracted image MFI was reported for evaluation of protein expression

As shown in FIG. 14, the in vitro expression of the 40_Aus and 16_Aus designs, combined with machine learning derived codon optimization algorithms, is roughly 3-fold higher than the wild-type counterpart (WT) with the benchmark codon optimization sequence.

### Example 6. Fusogenicity (Ability to Mediate Cell-Cell Fusion) of Influenza B HA Polypeptides

This example describes the functional assessment of the prefusion-stabilized designs in a fusion assay previously described in Milder et al., Proc Natl Acad Sci USA, 2022, 119(6):e2115379119. The hypothesis is that prefusion stabilized designs will show no, or less fusion, compared to the wild-type protein. Briefly, Vero cells were co-transfected with plasmid DNAs encoding various influenza HA polypeptides (e.g., the wild-type HA polypeptide from B/Austria/1359417/2021, the 40_Aus design, or the 16_Aus design) and a reporter GFP plasmid (to assess fused cells) using the Trans-IT transfection reagent in media containing trypsin (to allow for proper processing of HAG into HA1-HA2). After transfection, cells were incubated overnight at 37° C./5% CO₂ in a humidified incubator. Cells were imaged under a fluorescent microscope prior to the low pH incubation to set a baseline control. Cells were then exposed to pH 5.0 for 10 minutes, after which the media was aspirated and replaced with normal cell culture media. After 1 hour incubation at 37° C./5% CO₂ in a humidified incubator, cells were imaged again to determine the formation of syncytia (multinucleated fused cells), which would indicate that the transfected HA polypeptide was able to mediate cell fusion.

As shown in FIG. 15, the wild-type HA polypeptide from B/Austria/1359417/2021 (“B/Austria WT”) can mediate fusion and syncytia formation. The HAG polypeptide, which has a substitution in the HA1-HA2 cleavage site and thus is fusion deficient (“B/Aus HA0”), serves as a negative control for the assay and shows no fusion activity. The 16_Aus design (“B/Aus_16”) shows no fusion, comparable to HA0, while the 40_Aus design (“B/Austria_40”) shows reduced fusion compared to the wild-type counterpart. These results suggest that the 16_Aus and 40_Aus designs described herein are stabilized, to varying degrees, in the prefusion conformation.

### Example 7. Structural Characterization of Influenza B HA Polypeptides Using Negative Stain Electron Microscopy (nsEM)

Negative stain samples were prepared using 400 mesh copper grids (Electron Microscopy Sciences) with a thin carbon film. The grids were glow-discharged in air for 10 seconds with 25 W of power using the PELCO easiGlow (TED PELLA). Protein sample was incubated on a grid for one minute at room temperature. During staining, the grid was sequentially incubated on 4 drops, 30 μL each, of 2% uranyl formate solution for 5, 10, 15, and 20 seconds and blotted dry with #1 filter paper (Whatman). Images were collected on a Tecnai T12 microscope at 42,000× magnification and a defocus range of −1.5 to −3 μm. The nsEM images were processed using CRYOSPARC™ software, where particles were picked, extracted, and 2D classified.

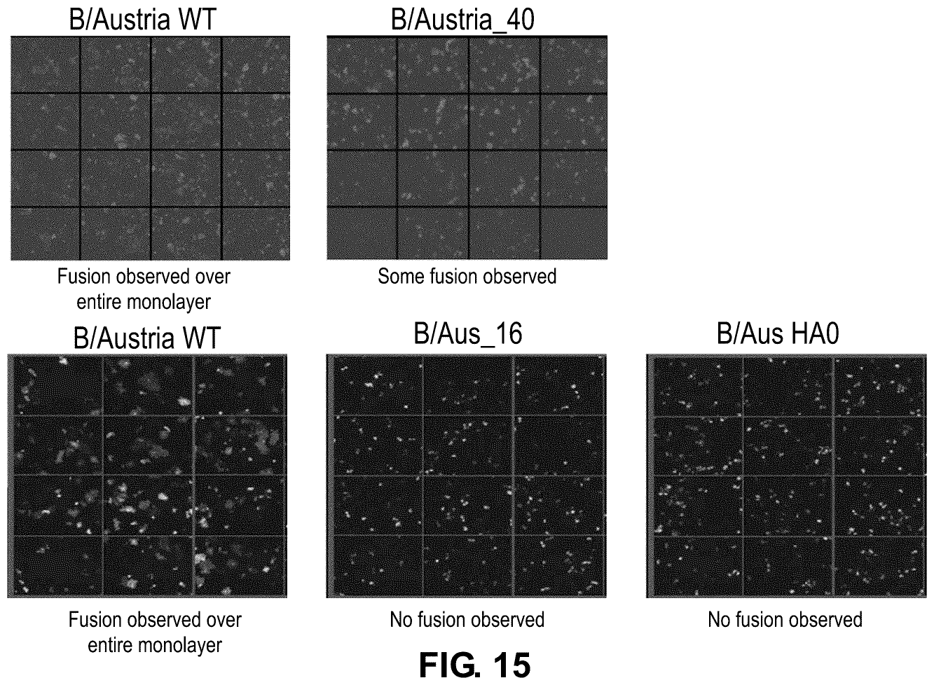

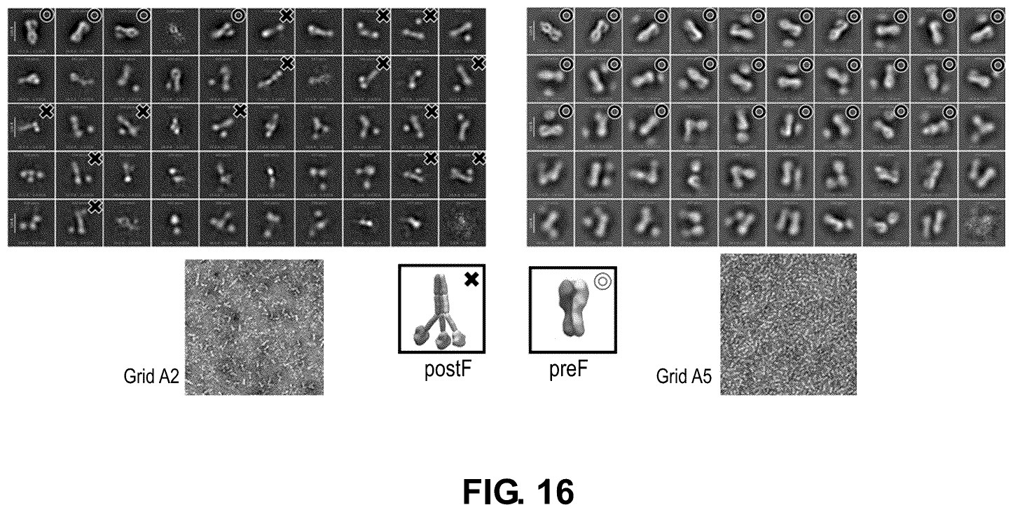

Representative results of the nsEM images after low pH exposure of the wild-type HA polypeptide from B/Austria/1359417/2021 and the 40_Aus design are shown in FIG. 16. As can be seen in FIG. 16, the 40_Aus design (right) retains a mostly prefusion conformation after low pH exposure, while the wild-type HA polypeptide (left) mostly shifts to postfusion as expected.

### Example 8. Immunogenicity of Influenza B HA Polypeptides Delivered as Lipid Nanoparticle (LNP) Formulated mRNA

This example describes the immunogenicity of representative modified influenza B HA polypeptides delivered as LNP-formulated mRNA in mice.

Immunogenicity was assessed using a prime-boost regimen in naïve mice, using a dose of 0.2 μg mRNA/LNP formulated HA per animal. In this study, 6-8-week-old BALB/C mice in groups of 6 were immunized by the intramuscular route with the indicated monovalent formulated HA polypeptide, encompassing the different design strategies (e.g., disulfide bonds, pH sensor KO, interphase stabilization, and proline stabilization), or a negative control of empty LNPs (without any mRNA). Mice were primed on day 0 and boosted on day 21 with the same test article and controls by the same immunization route. Animals were bled on days 20 and 35 to assess the neutralizing antibody titers against influenza B/Michigan/01/2021 strain, which is antigenically-like influenza B/Austria/1359417/2021 strain, by the hemagglutination inhibition (HAI) assay.

The HAI assay was conducted as follows. The sera samples were treated with receptor destroying enzyme (RDE) by diluting one-part serum with four-parts enzyme and incubated 18-20 hours in a 37° C. water bath. The enzyme was inactivated by 35-45-minute incubation at 56° C. Treated sera was diluted 2-fold for a dilution range of 1:10 to 1:5,120 for testing against the B/Michigan/01/2021 strain. The HAI assay was performed in V bottom 96-well plates using 4 HAU of chicken embryonated egg amplified virus and 0.5% turkey red blood cell (TBC) (serum starting dilution=1:10). The HAI titer was determined as the highest dilution of serum resulting in complete inhibition of hemagglutination and is reported by the dilution titer.

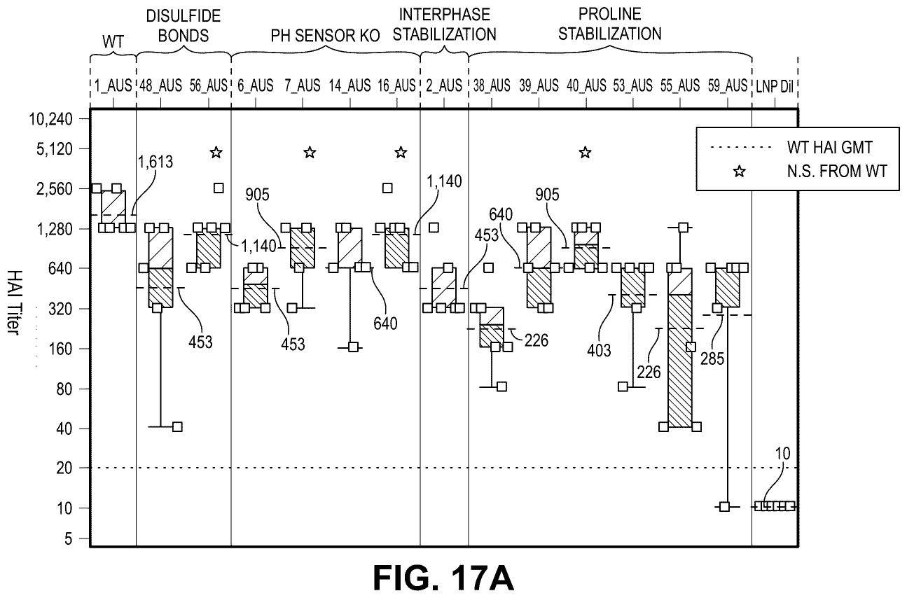

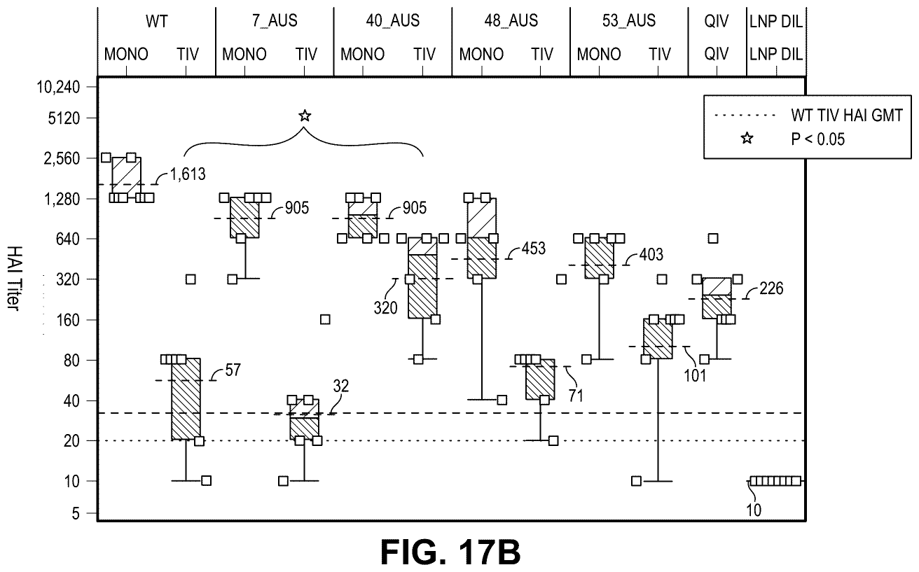

As shown in FIG. 17A, of the 13 modified influenza B HA polypeptides tested, 4 showed statistically similar (denoted by the star) HAI titers to the wild-type influenza B HA polypeptide (WT or “1_Aus”; geometrical mean titers (GMT) of the wild-type polypeptide shown by the dashed line), including the 40_Aus and 16_Aus designs. No responses were seen with empty LNPs as expected (“LNP dil”).

A selection of four of the 13 designs were also tested in the same study as trivalent influenza vaccines (TIV) formulations with the H1N1 component (A/WestVirginia/30/2022) and the H3N2 component (A/Darwin/06/2021) to more closely mimic the strain composition of licensed vaccines. As shown in FIG. 17B, there is a significant decrease in the HAI titers for the wild-type influenza B HA polypeptide (WT) when tested as part of the TIV formulation, compared to the monovalent. Similar drops are seen for the 7_Aus and 48_Aus designs, although the decrease is less dramatic for the 53_Aus design. While also exhibiting some decrease, the 40_Aus design has titers as a TIV formulation that are significantly higher than the wild-type TIV formulation.

### Example 9. Immunogenicity of Influenza B HA Polypeptides Delivered as Purified Recombinant Soluble Protein

This example describes the immunogenicity of one representative modified influenza B HA polypeptide delivered as purified recombinant soluble protein in mice.

To assess the robustness of the modified influenza B HA polypeptide designs disclosed herein on other platforms, such as recombinant protein platform, an immunogenicity study was conducted using a prime-boost regimen in 6-8-week-old BALB/C mice (6 per group), using escalating doses of 0.2 g and 1 μg of adjuvanted protein with AF03, and 4.5 μg of unadjuvanted protein per animal. Mice were primed on day 0 and boosted on day 21 with the same test article by the same immunization route. Animals were bled on days 20 and 35 to assess the neutralizing antibody titers against influenza B/Michigan/01/2021 strain, which is antigenically-like the influenza B/Austria/1359417/2021 strain, by the HAI assay as described above and a high content imaging-based micro-neutralization test (HINT) to assess neutralization titers against the B/Austria/1359417/2021 virus. The HINT assay was performed as follows. Briefly, serial 2-fold dilutions of receptor destroying enzyme (RDE)-treated sera from 1:40 to 1:40,960 were mixed with an equal volume of virus, about 1000 focus forming units (FFU), and incubated for 60 minutes at 37° C./5% CO₂ in a humidified incubator. After incubation, an MDCK SIAT1 cell suspension of 30,000 cells/well was added to the virus-sera mixture and incubated for about 22 hours at 37° C./5% CO₂ in a humidified incubator. The cell monolayers were fixed with methanol and prepared for staining. Wells were then incubated with anti-influenza monoclonal antibody against NP, followed by an Alexa FLUOR®488-conjugated secondary antibody. Cells were washed and plates scanned on CTL ImmunoSpot Cell Imaging v2. Counts from plate were transferred into SoftMax Pro 6 GxP software in the Sanofi Universal Exporter to calculate neutralization 50 (NT50) titers that achieves 50% foci reduction from sigmoidal curve (using the 4PL Sigmoidal dose-response [variable slope] function).

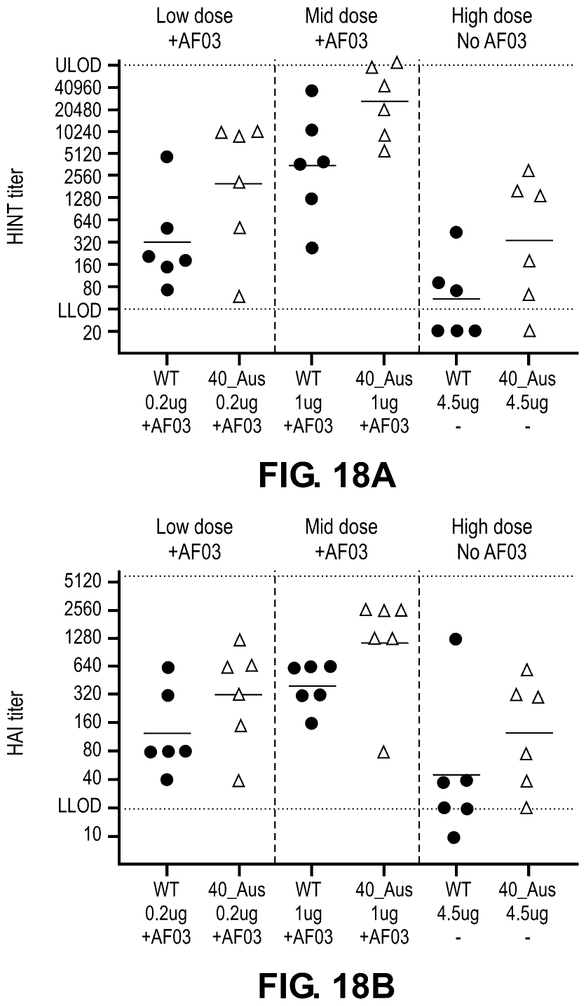

FIG. 18A shows that, at the adjuvanted low and mid dose (0.2 μg and 1 μg, respectively), the adjuvanted soluble protein of the 40_Aus design (“40_Aus”) induced roughly 4-fold higher titers than the adjuvanted wild-type (“WT”) protein. Similar results were observed in the unadjuvanted high dose of 4.5 μg, where the 40_Aus design also had 4-fold higher titers than the wild type. In the HAI assay shown in FIG. 18B, a very similar trend was observed, though the fold difference between the 40_Aus design and the wild type was in the 2-3-fold range. Taken together, these data indicate that the protein stabilization strategies proposed for mRNA could also be applicable to other vaccine platforms.

### Example 10. In Vitro Expression of Modified Influenza B HA Polypeptides in a Diverse Panel of B/Victoria Influenza Virus Strains

This example describes the in vitro expression of two representative modified influenza B HA polypeptides in a diverse panel of B/Victoria influenza virus strains.

To determine if the modified influenza B HA polypeptide designs based on B/Austria/1359417/2021 described in the above examples are transferrable to other B/Victoria influenza virus strains, a panel of antigenically diverse strains from the B/Victoria lineage was selected, corresponding modified HA polypeptides based on the 40_Aus design or the 16_Aus design were generated, and their expression level was compared to the expression level of the respective wild-type counterpart. The experiment was carried out as described in Example 5.

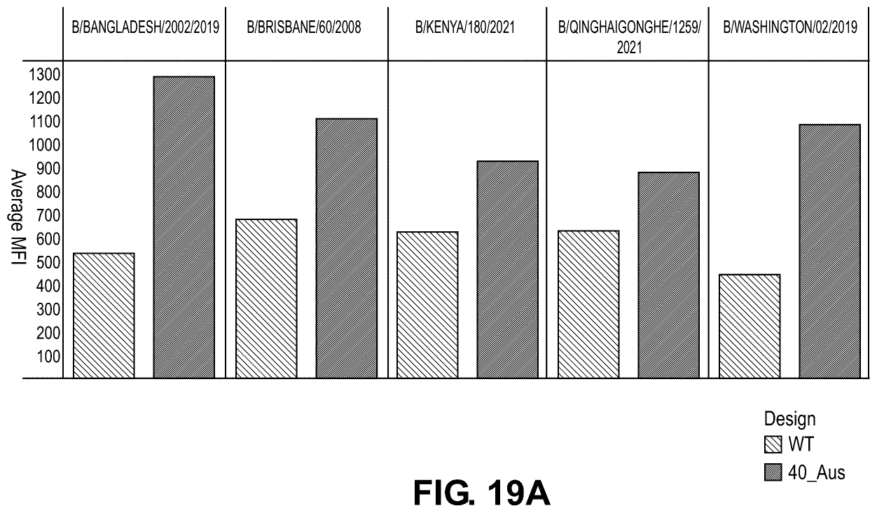

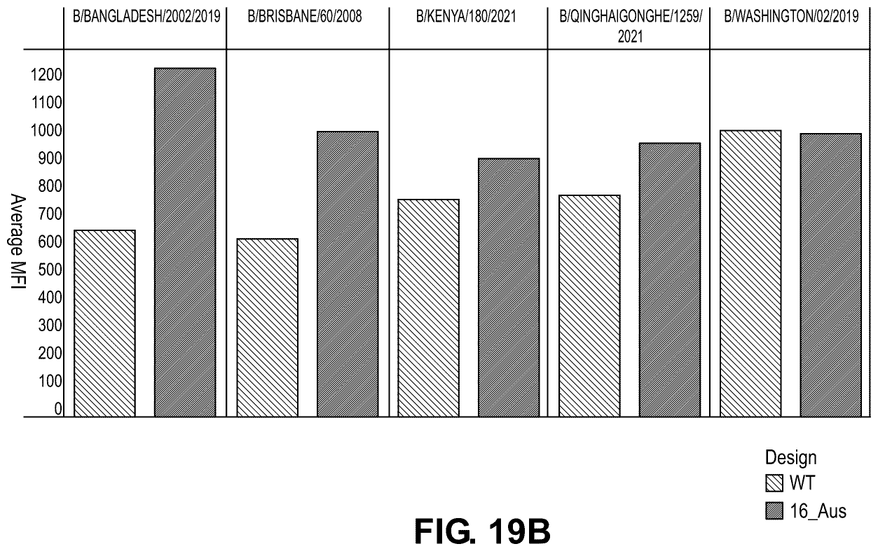

FIG. 19A and FIG. 19B show representative results using the designs on five different B/Victoria strains: B/Bangladesh/2002/2019, B/Brisbane/60/2008, B/Kenya/180/2021, B/Qinghaigonghe/1259/2021, and B/Washington/02/2019. FIG. 19A shows that, on the 5 strains tested, the 40_Aus design increased expression from about 50% to about 200%, which is similar to the increase previously observed using the B/Austria/1359417/2021 strain, with a highest improvement observed in the B/Bangladesh/2002/2019 strain. As shown in FIG. 19B, the increase in expression from the 16_Aus design also reached a maximum of about 2-fold for the B/Bangladesh/2002/2019 strain, while the other strains had modest improvement or similar expression level as the wild-type counterpart. Overall, the results support the transferability of the design strategies described herein to additional B/Victoria strains.

### Example 11. Assessment of Additional Modified Influenza B HA Polypeptides Based on A Combinatorial Approach

This example demonstrates the generation of additional modified influenza B HA polypeptides based on a non-exhaustive combinatorial approach for some of the designs described herein. The respective design ID, construct name (or “Combo design”), and designs that were combined to produce the combination design (“Parent designs”) for each additional modified influenza B HA polypeptides are provided in Table 8.

TABLE 8
B/Austria/1359417/2021 combo designs tested for expression and conformational
characterization (the numbering of the amino acid positions is as indexed by reference to the amino
acid sequence of SEQ ID NO: 2).

| Design ID | Combo design | Parent designs |
|---|---|---|
| 377_Aus | A428P-N434P_T347V | Aus-40_58 |
| 378_Aus | A428P-N434P_V419P | Aus-40_59 |
| 379_Aus | A458K_A428P-N434P | Aus-2_40 |
| 380_Aus | A458K_H381C_S399C | Aus-2_48 |
| 381_Aus | A458K_S399C_H473C | Aus-2_56 |
| 382_Aus | H381C_S399C_T347V | Aus-48_58 |
| 383_Aus | H381C_S399C_V419P | Aus-48_59 |
| 384_Aus | S399C_H473C_T347V | Aus-56_58 |
| 385_Aus | S399C_H473C_V419P | Aus-56_59 |
| 386_Aus | A428P-N434P_H381C-S399C | Aus-40_48 |
| 387_Aus | A428P-N434P_S399C-H473C | Aus-40_56 |
| 388_Aus | A428P-N434P_T347V_V419P | Aus-40_58_59 |
| 389_Aus | A458K_A428P-N434P_T347V | Aus-2_40_58 |
| 390_Aus | A458K_A428P-N434P_V419P | Aus-2_40_59 |
| 391_Aus | A458K_H381C_S399C_T347V | Aus-2_48_58 |
| 392_Aus | A458K_H381C_S399C_V419P | Aus-2_48_59 |
| 393_Aus | A458K_S399C_H473C_T347V | Aus-2_56_58 |
| 394_Aus | A458K_S399C_H473C_V419P | Aus-2_56_59 |
| 395_Aus | A458K_T347V_V419P | Aus-2_58_59 |
| 396_Aus | H381C_S399C_T347V_V419P | Aus-48_58_59 |
| 397_Aus | S399C_H473C_T347V_V419P | Aus-56_58_59 |
| 398_Aus | A428P-N434P_H381C-S399C_T347V | Aus-40_48_58 |
| 399_Aus | A428P-N434P_H381C-S399C_V419P | Aus-40_48_59 |
| 400_Aus | A428P-N434P_S399C-H473C_T347V | Aus-40_56_58 |
| 401_Aus | A428P-N434P_S399C-H473C_V419P | Aus-40_56_59 |
| 402_Aus | A458K_A428P-N434P_H381C-S399C | Aus-2_40_48 |
| 403_Aus | A458K_A428P-N434P_S399C-H473C | Aus-2_40_56 |
| 404_Aus | A458K_A428P-N434P_T347V_V419P | Aus-2_40_58_59 |
| 405_Aus | A458K_H381C_S399C_T347V_V419P | Aus-2_48_58_59 |
| 406_Aus | A458K_S399C_H473C_T347V_V419P | Aus-2_56_58_59 |
| 407_Aus | A428P-N434P_H381C-S399C_T347V_V419P | Aus-40_48_58_59 |
| 408_Aus | A428P-N434P_S399C-H473C_T347V_V419P | Aus-40_56_58_59 |
| 409_Aus | A458K_A428P-N434P_H381C-S399C_T347V | Aus-2_40_48_58 |
| 410_Aus | A458K_A428P-N434P_H381C-S399C_V419P | Aus-2_40_48_59 |
| 411_Aus | A458K_A428P-N434P_S399C-H473C_T347V | Aus-2_40_56_58 |
| 412_Aus | A458K_A428P-N434P_S399C-H473C_V419P | Aus-2_40_56_59 |
| 413_Aus | A458K_A428P-N434P_H381C-S399C_T347V_V419P | Aus-2_40_48_58_59 |
| 414_Aus | A458K_A428P-N434P_H381C- | Aus-2_40_56_58_59 |
| | S399C_T347V_V419P_K224M_T226V_H235L | |
| 415_Aus | A458K_A428P-N434P_S399C-H473C_T347V_V419P | Aus-2_40_56_58_59 |
| 416_Aus | A458K_A428P-N434P_S399C- | Aus-2_40_48_58_59 |
| | H473C_T347V_V419P_K224M_T226V_H235L | |

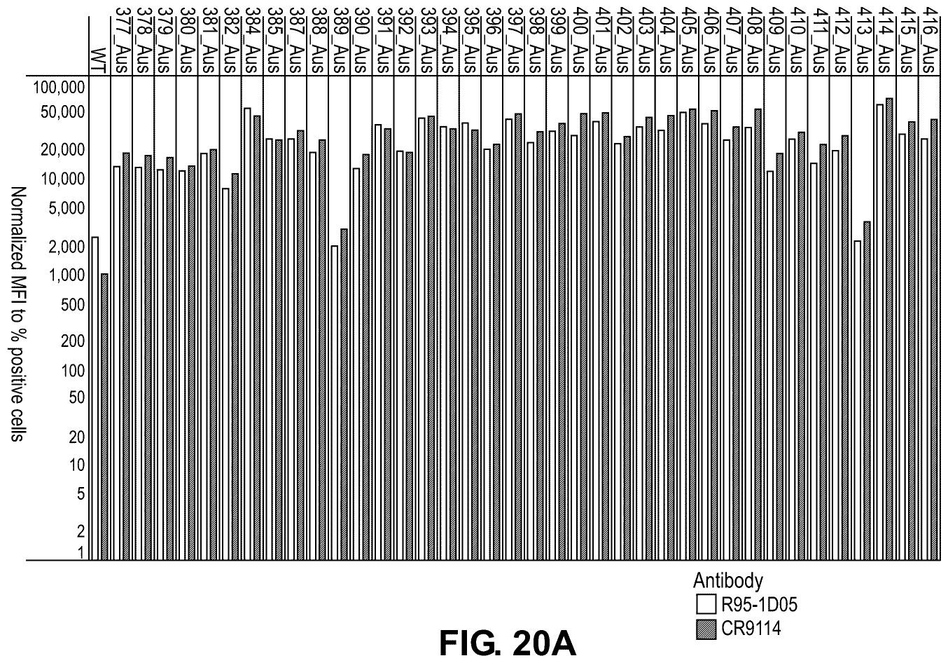

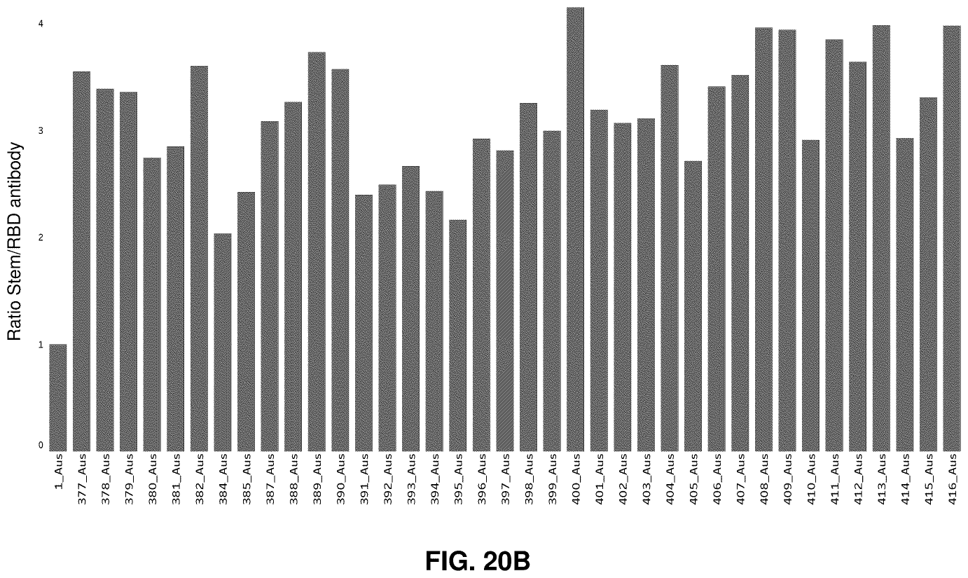

The combo designs were compared to the wild-type HA polypeptide from B/Austria/1359417/2021 to assess the effect of combining multiple designs on the conformation of the HA polypeptide. The experiment was performed as described in Example 4, except that a single transfection was performed. For simplicity, only the RBD antibody (R95-1D05) and the stem antibody (CR9114) were used for this screening. Results show that all of the combo designs screened showed similar, or higher, binding for both antibodies, and had higher stem/RBD ratios (ranging from about 2-4-fold) than the wild-type HA protein (FIG. 20A-20B). The results show that all of the combinatorial designs tested has a stem/RBD ratio higher than 2 (FIG. 20B).

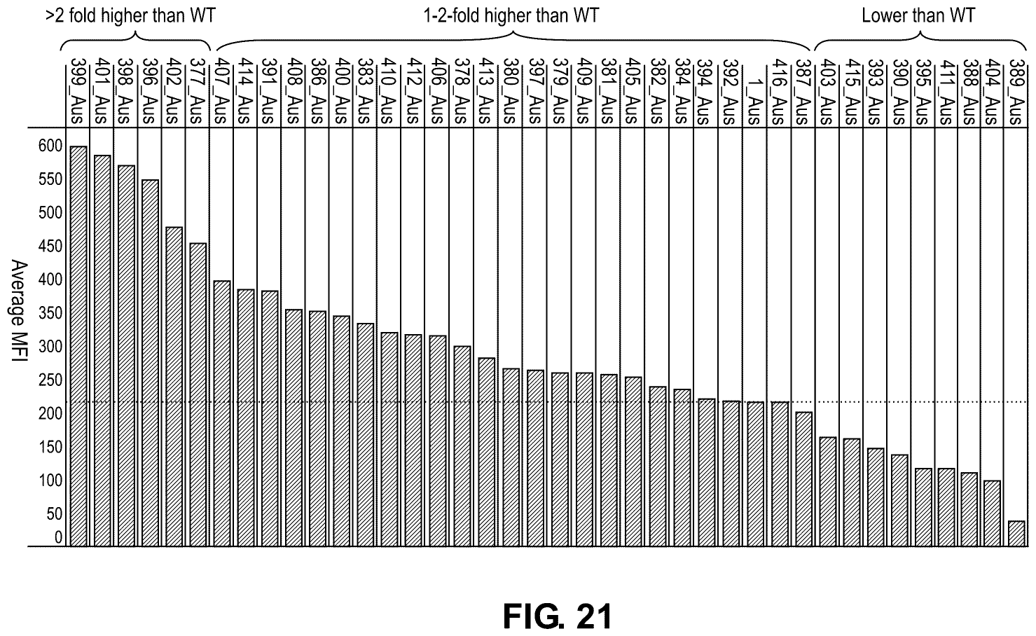

The combo designs were also compared to the wild-type HA polypeptide for total expression, to determine if combination of the substitutions could have a detrimental effect on expression. This experiment was performed as described in Example 5. The dashed line represents the average MFI obtained from the wild-type HA polypeptide from B/Austria/1359417/2021. Results show a range of responses, with a few designs showing lower expression than the wild type, the majority showing an increase no higher than 2-fold, and a few designs showing increases higher than 2-fold (FIG. 21).

While the foregoing disclosure has been described in some detail by way of illustration and example for purposes of clarity and understanding, it will be clear to one of ordinary skill in the art from a reading of this disclosure that various changes in form and detail can be made without departing from the true scope of the present disclosure and may be practiced within the scope of the appended claims. For example, all constructs, methods, and/or component features, steps, elements, or other aspects thereof can be used in various combinations.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or the entire group members are present in, employed in, or otherwise relevant to a given product or process. Furthermore, it is to be understood that the disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. In general, where embodiments or aspects of the present disclosure, is/are referred to as comprising particular elements, features, etc., certain embodiments or aspects consist, or consist essentially of, such elements, features, etc. For purposes of simplicity those embodiments have not in every case been specifically set forth in so many words herein. It should also be understood that any embodiment or aspect of the present disclosure can be explicitly excluded from the claims, regardless of whether the specific exclusion is recited in the specification.

All patents, patent applications, websites, other publications or documents, accession numbers and the like cited herein are incorporated by reference in their entirety for all purposes to the same extent as if each individual item were specifically and individually indicated to be so incorporated by reference.

1. An artificial messenger ribonucleic acid (mRNA) encoding an influenza B HA polypeptide, wherein the influenza B HA polypeptide comprises:
a) at least one proline substitution relative to a corresponding wild-type influenza B HA polypeptide, wherein the at least one proline substitution is at amino acid position 363, 366, 371, 372, 376, 380, 383, 390, 391, 393, 395, 397, 399, 421, 430, 431, 434, 436, and/or 490 as indexed by reference to the amino acid sequence of SEQ ID NO: 1;
b) at least two cysteine substitutions relative to a corresponding wild-type influenza B HA polypeptide, wherein the at least two cysteine substitutions are at amino acid positions 20 and 387, 35 and 408, 36 and 415, 37 and 411, 125 and 431, 127 and 431, 185 and 223, 186 and 224, 186 and 239, 188 and 241, 232 and 433, 233 and 434, 239 and 276, 346 and 465, 367 and 478, 378 and 397, 380 and 397, 383 and 401, 387 and 510, 394 and 507, 394 and 510, 396 and 510, 396 and 514, 401 and 475, 430 and 437, 430 and 438, and/or 430 and 439, as indexed by reference to the amino acid sequence of SEQ ID NO: 1;
c) at least one cavity filling amino acid substitution relative to a corresponding wild-type influenza B HA polypeptide, wherein the at least one cavity filling amino acid substitution is at amino acid position 460, 467, and/or 474 as indexed by reference to the amino acid sequence of SEQ ID NO: 1;
d) one or more interface stabilization amino acid substitutions relative to a corresponding wild-type influenza B HA polypeptide, wherein the one or more interface stabilization amino acid substitutions are at amino acid position 18, 121, 188, 226, 228, 408, 435, and/or 460 as indexed by reference to the amino acid sequence of SEQ ID NO: 1;
e) one or more pH sensor knock-out amino acid substitutions relative to a corresponding wild-type influenza B HA polypeptide, wherein the one or more pH sensor knock-out amino acid substitutions are at amino acid position 226, 228, 237, 239, 383, 388, 391, 401, 405, 408, 435, 460, 474 and/or 475 as indexed by reference to the amino acid sequence of SEQ ID NO: 1;
f) at least one amino acid substitution relative to a corresponding wild-type influenza B HA polypeptide, wherein the at least one amino acid substitution generates or disrupts a N-linked glycosylation motif in the influenza B HA polypeptide and is at amino acid position 28, 60, 62, 141, 143, 186, 187, 214, 216, 223, 224, 336, and/or 349 as indexed by reference to the amino acid sequence of SEQ ID NO: 1; and/or
g) at least one amino acid substitution relative to a corresponding wild-type influenza B HA polypeptide, wherein the at least one amino acid substitution is at amino acid position 157, 177, 218, and/or 257 as indexed by reference to the amino acid sequence of SEQ ID NO: 1.

2. The artificial mRNA of claim 1, wherein the influenza B HA polypeptide comprises:
a) two proline substitutions relative to a corresponding wild-type influenza B HA polypeptide, wherein the two proline substitutions are at amino acid positions 430 and 436 as indexed by reference to the amino acid sequence of SEQ ID NO: 1; or
b) five amino acid substitutions relative to a corresponding wild-type influenza B HA polypeptide, wherein the five amino acid substitutions are at amino acid positions 383, 401, 405, 408, and 475 as indexed by reference to the amino acid sequence of SEQ ID NO: 1.

3. The artificial mRNA of claim 2, wherein the influenza B HA polypeptide comprises amino acid substitutions:
a) A430P and N436P; or
b) H383M, S401V, A405V, K408M, and H475M,
as indexed by reference to the amino acid sequence of SEQ ID NO: 1.

4. The artificial mRNA of claim 1, wherein the influenza B HA polypeptide is from a B/Victoria influenza virus or influenza virus strain B/Austria/1359417/2021.

5. (canceled)

6. The artificial mRNA of claim 1, wherein:
a) the influenza B HA polypeptide comprises an amino acid sequence having at least about 90% sequence identity to the amino acid sequence of SEQ ID NO: 3 or SEQ ID NO: 5;
b) the influenza B HA polypeptide comprises the amino acid sequence of SEQ ID NO: 3 or SEQ ID NO: 5;
c) the artificial mRNA comprises a nucleic acid sequence having at least about 90% sequence identity to the nucleic acid sequence of SEQ ID NO: 4 or SEQ ID NO: 6; or
d) the artificial mRNA comprises the nucleic acid sequence of SEQ ID NO: 4 or SEQ ID NO: 6.

7-9. (canceled)

10. The artificial mRNA of claim 1, comprising:
a) a 5′-cap structure and/or a 3′-poly(A) sequence;
b) at least one chemically modified nucleotide and/or a phosphorothioate bond;
c) at least one chemically modified nucleotide comprising a pseudouridine, a 2′-fluoro ribonucleotide, or a 2′-methoxy ribonucleotide; and/or
d) a N1-methylpseudouridine.

11-12. (canceled)

13. A composition comprising the artificial mRNA of claim 1 encapsulated in a lipid nanoparticle (LNP).

14. The composition of claim 13, wherein the LNP comprises a cationic lipid, and wherein the cationic lipid optionally comprises or is OF-02, cKK-E10, GL-HEPES-E3-E10-DS-3-E18-1, GL-HEPES-E3-E12-DS-4-E10, GL-HEPES-E3-E12-DS-3-E14, (4-hydroxybutyl)azanediyl]di(hexane-6,1-diyl) bis(2-hexyldecanoate) (ALC-0315), or IM-001.

15. (canceled)

16. The composition of claim 14, wherein the LNP further comprises a polyethylene glycol conjugated (PEGylated) lipid, a cholesterol-based lipid, and a helper lipid, and wherein the PEGylated lipid optionally comprises or is 1,2-dimyristoyl-rac-glycero-3-methoxypolyethylene glycol-2000 (DMG-PEG2000), and/or the cholesterol-based lipid optionally comprises or is cholesterol, and/or the helper lipid optionally comprises or is dioleoyl-sn-glycero-3-phosphoethanolamine (DOPE).

17. (canceled)

18. The composition of claim 16, wherein:
a) the cationic lipid is present at a molar ratio between about 35% and about 55%;
b) the PEGylated lipid is present at a molar ratio between about 0.25% and about 2.75%;
c) the cholesterol-based lipid is present at a molar ratio between about 20% and about 45%; and
d) the helper lipid is present at a molar ratio between about 5% and about 35%, wherein all of the molar ratios are relative to the total lipid content of the LNP.

19. (canceled)

20. The composition of claim 19, wherein:
a) the artificial mRNA encodes the influenza B HA polypeptide of SEQ ID NO: 3, and wherein the LNP comprises GL-HEPES-E3-E12-DS-4-E10 at a molar ratio of about 40%, DMG-PEG2000 at a molar ratio of about 1.5%, cholesterol at a molar ratio of about 28.5%, and DOPE at a molar ratio of about 30%; or
b) the artificial mRNA encodes the influenza B HA polypeptide of SEQ ID NO: 5, and wherein the LNP comprises GL-HEPES-E3-E12-DS-4-E10 at a molar ratio of about 40%, DMG-PEG2000 at a molar ratio of about 1.5%, cholesterol at a molar ratio of about 28.5%, and DOPE at a molar ratio of about 30%.

21. (canceled)

22. The composition of claim 13, wherein the composition is an immunogenic composition.

23. An influenza B HA polypeptide comprising one or more amino acid substitutions relative to a corresponding wild-type influenza B HA polypeptide, wherein the one or more amino acid substitutions comprises:
a) two proline substitutions at amino acid positions 430 and 436 as indexed by reference to the amino acid sequence of SEQ ID NO: 1; or
b) amino acid substitutions at amino acid positions 383, 401, 405, 408, and 475 as indexed by reference to the amino acid sequence of SEQ ID NO: 1.

24. The influenza B HA polypeptide of claim 23, comprising amino acid substitutions:
a) A430P and N436P; or
b) H383M, S401V, A405V, K408M, and H475M,
as indexed by reference to the amino acid sequence of SEQ ID NO: 1.

25. The influenza B HA polypeptide of claim 23, wherein the influenza B HA polypeptide is from a B/Victoria influenza virus or influenza virus strain B/Austria/1359417/2021.

26. (canceled)

27. The influenza B HA polypeptide of claim 23, comprising:
a) an amino acid sequence having at least about 90% sequence identity to the amino acid sequence of SEQ ID NO: 3 or SEQ ID NO: 5; or
b) the amino acid sequence of SEQ ID NO: 3 or SEQ ID NO: 5.

28. (canceled)

29. A trimeric influenza B HA polypeptide complex, comprising three copies of the influenza B HA polypeptide of claim 23.

30. An artificial nucleic acid encoding the influenza B HA polypeptide of claim 23.

31. (canceled)

32. A vector comprising the artificial nucleic acid of claim 30.

33. (canceled)

34. A host cell comprising the vector of claim 32.

35. A composition comprising the influenza B HA polypeptide of claim 23, a trimeric influenza B HA polypeptide complex comprising three copies of said influenza B HA polypeptide, an artificial nucleic acid encoding said influenza B HA polypeptide, or a vector comprising an artificial nucleic acid encoding said influenza B HA polypeptide.

36. The composition of claim 35, wherein the composition is an immunogenic composition.

37. A vaccine comprising the composition of claim 22 and a pharmaceutically acceptable carrier.

38. A vaccine comprising the composition of claim 36 and a pharmaceutically acceptable carrier.

39. The vaccine of claim 37, wherein the vaccine is an mRNA vaccine, and wherein:
a) the vaccine further comprises an mRNA encoding an influenza H3 HA polypeptide and an mRNA encoding an influenza H1 HA polypeptide; or
b) the vaccine further comprises an mRNA encoding an influenza H3 HA polypeptide, an mRNA encoding an influenza H1 HA polypeptide, an mRNA encoding an influenza N2 neuraminidase (NA) polypeptide, an mRNA encoding an influenza N1 NA polypeptide, and an mRNA encoding an influenza NA polypeptide from an influenza B/Victoria lineage.

40. (canceled)

41. The vaccine of claim 38, wherein the vaccine is a recombinant vaccine, and wherein:
a) the vaccine further comprises an influenza H3 HA polypeptide and an influenza H1 HA polypeptide; or
b) the vaccine further comprises an influenza H3 HA polypeptide, an influenza H1 HA polypeptide, an influenza N2 NA polypeptide, an influenza N1 NA polypeptide, and an influenza NA polypeptide from an influenza B/Victoria lineage.

42. (canceled)

43. A method of immunizing a subject or reducing one or more symptoms of an influenza B virus infection, the method comprising administering to the subject in need thereof the vaccine of claim 37.

44. The method of claim 43, wherein:
a) the method prevents an influenza B virus infection in the subject, decreases the subject's likelihood of getting an influenza B virus infection, or reduces the subject's likelihood of getting serious illness from an influenza B virus infection;
b) the subject is a human;
c) the subject is a human of 6 months of age or older, less than 18 years of age, at least 6 months of age and less than 18 years of age, at least 18 years of age and less than 65 years of age, at least 6 months of age and less than 5 years of age, at least 5 years of age and less than 65 years of age, at least 60 years of age, or at least 65 years of age; and/or
d) the vaccine is administered intramuscularly, intradermally, subcutaneously, intravenously, intranasally, by inhalation, or intraperitoneally.

45-48. (canceled)

49. An in vitro method of preparing a trimeric influenza B HA polypeptide complex, the method comprising culturing the host cell of claim 34 in a cell culture medium, and expressing the trimeric influenza B HA polypeptide complex, wherein the method optionally comprises a step of purifying the trimeric influenza B HA polypeptide complex from the cell culture medium.

50. (canceled)

51. A method of immunizing a subject or reducing one or more symptoms of an influenza B virus infection, the method comprising administering to the subject in need thereof the vaccine of claim 38.


SEQUENCE LISTING (10 sequences)

SEQ ID NO: 1 - protein, 584 aa, Influenza B virus
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 2 - protein, 582 aa, Influenza B virus
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 3 - protein, 582 aa, synthetic construct
  source 1..582: note = Modified influenza B HA polypeptide
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGPMDELHPEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 4 - RNA, 1752 nt, synthetic construct
  source 1..1752: mol_type = other RNA; note = Codon-optimized mRNA sequence encoding the influenza B HA polypeptides
atgaaagcaatcatagtgctgctgatggtggtgactagcaatgccgataggatctgcactggaatcaccagcagcaatagtccccacgtggtgaagaccgctacccagggggaggtgaacgtgacaggggtgatccccctgaccaccacccccaccaagagccacttcgccaacctgaagggcacagaaacccgggggaagctgtgccctaagtgcctcaattgtacagacctcgatgtggccctggggcgccccaagtgcactgggaagatccccagtgcacgggtgtccatcctgcacgaggtgcggccagtgacctctggctgctttcctatcatgcacgataggaccaagatccggcagctgcccaacctgctgaggggctacgagcacgtgcgcctcagcactcacaacgtgatcaatacggaggacgcacctggtggaccctacgagatcggcaccagcggatcttgtctaaacatcaccaatgggaaagggttcttcgccaccatggcctgggccgtgcccaagaacaagaccgccaccaaccccctgacaattgaggtgccctacatctgcacagaggaagaggaccagatcaccgtctggggcttccactctgacgacgaaacccagatggcccgcctgtatggtgactccaagcctcagaagttcacttctagcgccaatggagtcaccacacactatgtgagccagattggggggttccccaaccagaccgaggatgggggactcccccagtctggccgcatcgtggtggactatatggtgcaaaagagcggcaagacgggcaccatcacctaccagcggggcatcctgctgccccagaaggtgtggtgcgcgtctggcaaatctaaggtgatcaagggcagcctccccctgatcggggaggctgactgcctgcacgagaagtatggcggactgaacaagtccaagccatactacaccggggagcatgccaaggccattggcaactgccccatctgggtgaagacccccctgaagctggccaacggcaccaagtacaggccacccgccaagctgctcaaggagcgcgggttcttcggcgccatcgccggcttcctggagggaggctgggagggcatgatcgctgggtggcacgggtacaccagccacggcgcccacggggtggccgtggctgctgacctgaagtccacccaggaggcgatcaacaagatcaccaagaacctgaactccttgagcgagctggaggtgaagaatctgcagcggctgtccggcccaatggacgagctgcacccagagattctggagctggacgagaaggtggacgatctgagagctgataccatcagctctcagatcgagctggctgtgctcctgagcaacgagggcatcatcaacagcgaggatgagcacctcctggctctggagcgcaagctgaagaagatgctgggccccagcgccgtcgagatcggtaacggctgcttcgaaaccaagcacaagtgcaaccagacttgtctggaccggatcgcagccggcaccttcgacgctggggagttcagcctccccaccttcgacagcctgaacatcaccgccgccagcctgaacgacgacgggctggacaaccacaccatcctgctgtactacagtactgcagccagcagcctggccgtgacactgatgatcgccatcttcgtggtgtacatggtgtcacgggacaatgtgagctgcagtatctgcctgtaataa

SEQ ID NO: 5 - protein, 582 aa, synthetic construct
  source 1..582: note = Modified influenza B HA polypeptide
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWMGYTSHGAHGVAVAADLKVTQEVINMITKNLNSLSELEVKNLQRLSGAMDELHNEILELDEKVDDLRADTISSQIELAVLLSNEGIINSEDEMLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 6 - RNA, 1752 nt, synthetic construct
  source 1..1752: mol_type = other RNA; note = Codon-optimized mRNA sequence encoding the influenza B HA polypeptides
atgaaagcaatcatagtgctgctgatggtggtgactagcaatgccgacaggatctgcaccggcatcacctcgtccaactccccccacgtggtgaagaccgccacgcagggggaggtgaatgtgacgggggtgatccccctgacgaccacccccaccaagtcgcacttcgccaacctgaagggcactgagacgcgcggcaagctgtgccctaagtgcctcaactgcacggacctggacgtggccctggggaggcccaagtgcacgggcaagatccccagcgccagggtgtccatcctgcacgaggtgaggccggtcacgtccgggtgcttccccatcatgcacgacaggaccaagatccggcagctgcctaatcttctgcgcggttacgagcacgtgcgcctcagcacgcacaacgtgatcaacacggaggatgcccccggcggaccttacgagatcggcacctcaggcagctgcctaaatatcaccaacggcaaaggcttctttgccaccatggcatgggccgtcccaaagaataagacggccacaaaccctctaaccatcgaggtgccctacatctgcactgaggaagaggatcagataactgtgtggggcttccactcggatgacgaaacccagatggcacgcctgtatggcgatagtaagccacagaaatttacatcatccgccaatggcgtgacaacacattacgtgagtcagatcggcggcttccccaatcaaacagaagatggcggcctgccacagagcggccgcatcgtcgtggactatatggtccaaaaatccggaaaaacgggcaccatcacctatcagaggggcattctgctccctcagaaggtgtggtgcgccagtgggaaaagtaaagtgatcaaaggctcactgcctctgatcggggaggccgactgtctgcacgaaaagtatggaggcttgaataaatccaagccctattacacaggcgaacacgccaaggcgattggaaattgccccatctgggtgaagaccccactgaagctggcgaatggcaccaaatataggccaccagccaagctcctcaaagagaggggcttctttggagccatcgccggcttcttggaaggcggctgggagggcatgatcgcaggttggatgggctatactagccatggcgcccacggcgtagccgtggccgccgatctaaaagttacacaggaggttataaatatgattacaaaaaatctaaacagcctaagtgaactggaggtaaaaaacctccagaggctgtccggggcaatggacgagctgcacaacgagattctggaactggacgagaaggtggacgaccttagggccgacaccatttcgtcacagatcgagctcgctgtgctgctgtccaatgagggcatcatcaattcagaagatgagatgctcctcgccctggaaagaaagctgaaaaaaatgctgggcccatccgcagtggagatcggcaacgggtgtttcgaaacaaagcataaatgtaaccagacatgtctggacagaattgcagcagggacattcgatgccggcgaattttcgctgccaaccttcgattcactgaacatcactgccgccagcctcaatgatgatgggttagataaccacaccatcctgctgtattattcaacagcagcctcgagccttgccgtgacactcatgatcgccatcttcgtggtgtacatggtgtcacgtgataacgtgtcgtgctccatttgtctgtaataa

SEQ ID NO: 7 - RNA, 11 nt, synthetic construct
  source 1..11: mol_type = other RNA; note = 5' UTR sequence
gggatcctacc

SEQ ID NO: 8 - RNA, 140 nt, synthetic construct
  source 1..140: mol_type = other RNA; note = 5' UTR sequence
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 9 - RNA, 100 nt, synthetic construct
  source 1..100: mol_type = other RNA; note = 3' UTR sequence
cgggtggcatccctgtgacccctccccagtgcctctcctggccctggaagttgccactccagtgcccaccagccttgtcctaataaaattaagttgcatc

SEQ ID NO: 10 - RNA, 110 nt, synthetic construct
  source 1..110: mol_type = other RNA; note = poly(A) tail sequence
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa